(19) United States
(12) Patent Application Publication
(10) Pub. No.: US 2026/0286298 A1
(43) Pub. Date: Sep. 24, 2026

(54) ENGINEERED CELL PREPARATIONS FOR TREATMENT OF HEMOPHILIA

(71) Applicant: Be Biopharma, Inc., Cambridge, MA (US)

(72) Inventors: Timothy James Mullen, Cambridge, MA (US); Hanlan Liu, Cambridge, MA (US); Anja Fides Hohmann, Somerville, MA (US); Sean Philip Leary Arlauckas, Boston, MA (US); Swati Singh, Acton, MA (US); Sean Patrick Keegan, North Andover, MA (US); Richard A. Morgan, Moultonborough, NH (US)

(21) Appl. No.: 19/475,886

(22) PCT Filed: Apr. 16, 2024
(86) PCT No.: PCT/US24/24829

§ 371 Date: Oct. 16, 2025

Related U.S. Application Data
(60) Provisional application No. 63/459,986, filed on Apr. 17, 2023.
(60) Provisional application No. 63/502,639, filed on May 16, 2023.
(60) Provisional application No. 63/592,495, filed on Oct. 23, 2023.
(60) Provisional application No. 63/608,114, filed on Dec. 8, 2023.
(60) Provisional application No. 63/623,756, filed on Jan. 22, 2024.
(60) Provisional application No. 63/553,520, filed on Feb. 14, 2024.

(51) Int. Cl. C12N 5/0781; A61K 35/17; C12N 9/22; C12N 9/64; C12N 13/00; C12N 15/11; C12N 15/86; C12N 15/87; C12N 15/90; G01N 33/569

(52) U.S. Cl. CPC C12N 5/0635; A61K 35/17; C12N 9/226; C12N 9/644; C12N 13/00; C12N 15/11; C12N 15/86; C12N 15/87; C12N 15/907; C12Y 304/21022; G01N 33/56972; C12N 2310/20; C12N 2501/2302; C12N 2501/231; C12N 2501/2315; C12N 2510/00; C12N 2750/14143

(57) ABSTRACT

The present disclosure provides methods and compositions for culturing, engineering, and administering engineered B lineage cell populations that produce Factor IX (FIX).

### CROSS-REFERENCE TO RELATED APPLICATION

This application claims the benefit of U.S. Provisional Applications 63/459,986, filed Apr. 17, 2023, 63/502,639, filed May 16, 2023, 63/592,495, filed Oct. 23, 2023, 63/608,114, filed Dec. 8, 2023, 63/623,756, filed Jan. 22, 2024, and 63/553,520, filed Feb. 14, 2024, the contents of each of which are hereby incorporated by reference in their entireties.

### BACKGROUND

Cell-based therapeutics are an emerging class of medicine that make use of innate cellular machinery to combat disease. Unlike traditional treatment methods, cell therapies make use of cellular localization, migration, and proliferation within the body, which can translate to improved biodistribution and targeted delivery of therapeutics. Cell-based therapies have potential applications for a broad range of diseases, including those that have proved intractable or difficult to manage with traditional treatment options.

### SUMMARY OF THE INVENTION

The present disclosure encompasses, among other things, compositions and methods comprising modified immune cells (e.g., B lineage cells). The present disclosure also encompasses, among other things, nucleic acid constructs comprising one or more nucleic acid sequences encoding Factor IX (FIX) described herein and methods of producing the same. The present disclosure provides, inter alia, FIX expressing engineered B lineage cell populations.

In some embodiments, the present disclosure provides methods of preparing an engineered B lineage cell population that express Factor IX (FIX), the method comprising the steps of: (a) isolating primary B cells so that a primary B cell population is obtained, (b) activating the primary B cell population, and (c) during or after the activating step, engineering the primary B cell population by integrating a transgene encoding FIX at a site selected from a CCR5 site, a IgH site, and a JCHAIN site, thereby generating an engineered B lineage cell population. In some embodiments, the method further comprises a step of expanding an engineered B lincage cell population.

Among other things, in one aspect, the present disclosure provides methods of differentiating an engineered B lineage cell population into a population of plasmablasts. In some embodiments, a differentiation step comprises contacting an engineered B lineage cell population with culture media comprising: (a) IL-2, (b) IL-6, (c) IL-10, and/or (d) IL-15.

Among other things, in one aspect, the present disclosure provides methods of differentiating an engineered B lineage cell population into a population of plasma cells. In some embodiments, a differentiation step comprises contacting a population of plasmablasts with culture media comprising: (a) IL-6, (b) IL-15, and/or (c) IFN-alpha-2-beta (IFNα-2B).

In some embodiments, the present disclosure provides methods of engineering B lineage cell populations. In some embodiments, the method further comprises a step of transferring an engineered B lineage cell population to cell culture media without human or bovine serum for about 24 hours. In some embodiments, the step of engineering further comprises introducing a donor construct comprising a transgene into the primary B cell population.

In some embodiments, a donor construct comprises: (a) a 5′ homology arm that is at least 95% identical to a sequence 5′ to a double-stranded break site, and (b) a 3′ homology arm that is at least 95% identical to a sequence 3′ to a double-stranded break site. In some embodiments, a 5′ homology arm comprises or is: (i) a length about 450-850 base pairs in size, (ii) a PAM site or absence of a PAM site, and/or (iii) symmetrical or asymmetrical in length with the 3′ homology arm. In some embodiments, a 3′ homology arm comprises or is: (i) a length about 450-850 base pairs in size, (ii) a PAM site or absence of a PAM site, and/or (iii) symmetrical or asymmetrical in length with the 5′ homology arm. In some embodiments, a 5′ homology arm has at least 95% identity with one or more of SEQ ID NOs: 10-13. In some embodiments, a 3′ homology arm has at least 95% identity with one or more of SEQ ID NOS: 14-18.

In some embodiments, a donor construct comprises a sequence with at least 95% identity with one or more of SEQ ID NOs: 19-21. In some embodiments, a donor construct further comprises one or more of: (a) a promoter sequence selected from SEQ ID NOs: 22, (b) a nucleic acid sequence encoding for Factor IX selected from SEQ ID NOs: 23, and (c) a polyA sequence selected from SEQ ID NOs: 24. In some embodiments, a donor construct is or comprises an adeno-associated viral (AAV) vector.

In some embodiments, a step of engineering comprises contacting the primary B cell population with a targeted nuclease capable of introducing double-stranded breaks. In some embodiments, a targeted nuclease is or comprises a CRISPR-associated (Cas) protein, zinc finger nuclease (ZFN), transcription activator-like effector-based nuclease (TALEN), or meganuclease. In some embodiments, a Cas protein is or comprises Cas9, Cas12a, or Cas13a, or a variant thereof. In some embodiments, a Cas protein is complexed with a guide RNA (gRNA). In some embodiments, a gRNA is a single guide RNA (sgRNA). In some embodiments, a gRNA comprises at least 95% identity with any one of SEQ ID NOs: 25-29.

In some embodiments, a step of engineering further comprises electroporation of the primary B cell population, and/or transduction of the primary B cell population with a donor construct. In some embodiments, electroporation is performed on a composition comprising: (a) a Cas protein complexed with the gRNA, and (b) a primary B cell population.

In another aspect, the present disclosure provides sites and transgenes for engineering of B lineage cell populations. In some embodiments, a CCR5 site has at least 95% identity with SEQ ID NO: 25. In some embodiments, a JCHAIN site has at least 95% identity with one or more SEQ ID NOs: 26-28. In some embodiments, an IgH site has at least 95% identity with SEQ ID NO: 29. In some embodiments, a transgene has at least 95% identity with SEQ ID NO: 23.

In another aspect, the present disclosure provides populations of genetically modified B lineage cells. In some embodiments, a population of genetically modified B lineage cells comprises a transgene sequence encoding a FIX protein, wherein a transgene is expressed from an endogenous CCR5 locus. In some embodiments, a population of genetically modified B lineage cells express FIX protein and at least partially disrupt expression of endogenous CCR5. In some embodiments, a population of genetically modified B lineage cells comprises a transgene sequence encoding a FIX protein, wherein a transgene is expressed from an endogenous JCHAIN locus. In some embodiments, a population of genetically modified B lineage cells express FIX protein and at least partially disrupt expression of endogenous JCHAIN. In some embodiments, a population of genetically modified B lineage cells comprises a transgene sequence encoding a FIX protein, wherein a transgene is expressed from an endogenous IgH locus. In some embodiments, a population of genetically modified B lineage cells express FIX protein and at least partially disrupt expression of endogenous IgH. In some embodiments, a population of genetically modified B lineage cells is or comprises a population of genetically modified plasma cells. In some embodiments, a population of genetically modified B lineage cells is or comprises a population of plasmablasts. In some embodiments, a population of genetically modified B lineage cells is or comprises a population of plasma cell precursors.

In another aspect, the present disclosure provides a pharmaceutical composition comprising one or more B lineage cells selected from a population of genetically modified B lineage cells. In some embodiments, a pharmaceutical composition comprises one or more B lineage cells selected from a population of genetically modified B lineage cells and further comprises one or more pharmaceutically acceptable excipients. In some embodiments, a method of administering a pharmaceutical composition, wherein the pharmaceutical composition comprises: (a) one or more B lineage cells selected from a population of genetically modified B lineage cells of any aspect or embodiment described herein, and (b) one or more pharmaceutically acceptable excipients, wherein the pharmaceutical composition is administered to a subject.

In another aspect, the present disclosure provides methods of treating a disease, disorder, or condition in a subject, the method comprising administering to the subject a therapeutically effective amount of a pharmaceutical composition, thereby treating the disease, disorder, or condition in the subject. In some embodiments, a pharmaceutical composition is administered intravenously. In some embodiments, a pharmaceutical composition is administered to an adult subject. In some embodiments, a pharmaceutical composition is administered to a pediatric subject. In some embodiments, a subject has hemophilia B.

In another aspect, the present disclosure provides methods of characterizing a population of genetically modified B lineage cells, the method comprising assessing one or more of (a)-(g) using an assay: (a) presence of a CD38 marker, (b) presence of a CD138 marker, (c) presence of at least 50% of a CD27 marker, (d) secretion of at least 0.5 μg/cell/day of IgG, (e) secretion of at least 2.5 μg/cell/day of IgM, (f) secretion of at least 0.5 μg/cell/day of IgD, and (g) secretion of at least 0.1 μg/cell/day of a FIX protein. In some embodiments, an assay comprises or is one or more of: fluorescence-activated cell sorting (FAC-sort), Western Blot, flow cytometry, enzyme-linked immunosorbent spot assay (ELISpot), and enzyme-linked immunosorbent assay (ELISA). In some embodiments, one or more B lineage cells selected from a population of genetically modified B lineage cells engraft within bone marrow of a subject. In some embodiments, a method of monitoring engraftment of genetically modified B lineage cells within a subject, comprising one or more of: bioluminescence, ELISpot, flow cytometry, and enzyme-linked immunosorbent assay.

### BRIEF DESCRIPTION OF THE DRAWINGS

The drawings are for illustration purposes only, not for limitation.

FIG. 1 shows AAV6 viral vectors comprising the following expression cassettes: p00573 (AMT061), p00576 (Zero CG.AH), and p00577 (Zero CG.SS). Each expression cassette comprises 5′ and 3′ homology arms (HAs) for integration, an MND promoter, and BGH poly-adenylation (polyA) sequence. Each of the expression cassettes contains a different polynucleotide encoding for FIX as described herein: AMT061, Zero CG.AH, and Zero CG.SS.

FIG. 2A demonstrates the following from left to right via ELISA assay: immunoglobulin G production in non-engineered B lineage cells (IgG-), IgG production in engineered B lineage cells (IgG SV), and Factor IX production in engineered B lineage cells (FIX SV). FIG. 2B demonstrates the ratio of Factor IX to Immunoglobulin G in engineered B lineage cells.

FIG. 3A shows graphs of Factor IX production measured via ELISA in B lineage cell populations engineered with the exemplary FIX expression cassettes and either CCR5-targeting guide RNA (gCCR5_232) or JCHAIN-targeting guide RNA (gJCHAIN_2). FIG. 3B shows graphs of percent homology-directed repair (HDR) as measured by droplet digital polymerase chain reaction (ddPCR) for the aforementioned B lineage cell populations engineered with expression cassettes as described herein. FIG. 3C shows graphs of FIX production normalized to percent HDR for these engineered B lineage cell populations.

FIG. 4A shows graphs of Factor IX secretion measured via ELIspot in B lineage cell populations engineered with the exemplary FIX expression cassettes and either CCR5-targeting guide RNA (gCCR5_232) or JCHAIN-targeting guide RNA (gJCHAIN_2). FIGS. 4B and 4C show graphs of Immunoglobulin G (IgG) and Immunoglobulin M secretion, respectively, measured via ELIspot in the aforementioned engineered B lineage cell populations.

FIG. 5A demonstrates the gating scheme through flow cytometry of engineered B cell lineage populations for plasma cells. FIG. 5B shows content of plasma cells in engineered B lineage cell populations with different expression cassettes and conditions disclosed in the present application.

FIG. 6A demonstrates FIX specific activity of B lineage cell populations engineered with p00573 expression cassette that were cultured overnight with either insoluble vitamin K1 (vitK1) or vitamin K1 conjugated to BSA (control, BSA carrier). FIG. 6B demonstrates FIX specific activity of B lineage cell populations that were engineered with (left two bars) or without FBS for 24 hours post editing (serum variation, right two bars). Engineered B lineage cell populations were further cultured overnight with either insoluble vitamin K1 (vitK1) or soluble vitamin K3 (vitK3) prior to FIX activity assay as described herein. FIG. 6C demonstrates FIX activity as measured by ELISA for B lineage cell populations that were engineered with (left bar of each pairing) or without FBS for 24 hours post editing (serum variation, right bar of each pairing) and additionally treated with vitamin K overnight before measurements.

FIG. 7 shows measurements of human Factor IX (huFIX) over time for mice after administration of two donors of B lineage cell populations (LKP22061 and LKP22064) that were engineered with the methods and expression cassettes as disclosed herein.

FIG. 8A demonstrates percentage HDR as measured by ddPCR for B lineage cell populations engineered with expression cassettes as described herein targeting the CCR5 locus. FIG. 8B shows FIX secretion measured via ELIspot in B lineage cell populations engineered with the exemplary FIX expression cassettes with CCR5-targeting guide RNA (gCCR5_232). FIG. 8C demonstrates FIX specific activity of B lineage cell populations engineered with expression cassettes disclosed herein that were cultured overnight with either vitamin K3 or media (control). FIG. 8D demonstrates activated partial thromoboplastin clotting time (aPPT) to quantify enzymatic activity of secreted FIX from engineered B lineage cells cultured in the presence or absence of Vitamin K.

FIG. 9A shows measurements of human FIX (huFIX) over time for mice after administration of two donors of B lineage cell populations that were engineered with methods and expression cassettes as disclosed herein. FIG. 9B shows measurements of human immunoglobulin G (hIgG) and human immunoglobulin M (hIgM) over time for mice after administration of two donors of B lineage cell populations that were engineered with methods and expression cassettes as disclosed herein.

FIG. 10 shows measurements of human Factor IX (huFIX) and human Ig (hIg) over time for mice after administration of a B lineage cell populations engineered with the methods and expression cassettes as disclosed herein. Specific activity of expressed Factor IX was measured for isolated and pooled samples collected on indicated days.

FIG. 11A shows measurements of antibody (human immunoglobulin G (HuIgG)) levels over time for mice after a single administration of B lineage cell populations engineered with methods and expression cassettes as disclosed herein. FIG. 11B shows measurements of HuIgG levels over time in mice after a single administration B lineage cell populations engineered with methods and expression cassettes as disclosed herein. In some embodiments, engineered B lineage cell populations exhibited sustained and long-term secretion in vivo of proteins (e.g., antibodies, such as IgG) described herein (e.g., for at least 2 weeks, at least 4 weeks, at least 6 weeks, at least 8 weeks, at least 10 weeks, at least 12 weeks, at least 14 weeks, at least 16 weeks, at least 18 weeks, or longer).

FIG. 12A shows a schematic for engineering of NOG-IL6 mice with B lineage cell populations (e.g., plasma cell populations) to express Factor IX (F9) from an endogenous CCR5 locus. FIG. 12B shows average picograms of integrated Factor IX human DNA (hDNA) expressed in mouse femurs and spinal columns after collection on day 1 (baseline, no treatment) and day 28 of treatment. FIG. 12C shows relative Factor IX hDNA levels in indicated tissues on day 1 (baseline, no treatment) and day 28 of treatment.

FIG. 13 shows levels of human Factor IX (hFIX), human Ig (hIg), and human IgM (hIgM) in mouse plasma collected on indicated days from mice treated with an initial dose of engineered B lineage cell populations at day 1 and a second dose (redose) at day 49.

FIG. 14 shows percentage change in body weight for mice treated with vehicle only or with a dose of 20×10⁶ viable engineered plasma cells from indicated donor.

FIG. 15 shows IgG, IgM, and huFIX levels in plasma of mice treated with plasma cell populations engineered for expression of huFIX. Samples were collected on indicated day post-transfer of engineered plasma cell populations (BCMs).

FIG. 16A shows IgG, IgM, and huFIX levels in plasma of mice treated with plasma cell populations engineered for expression of huFIX and administered directly (black data points) or cryopreserved and administered after thawing (blue data points). Samples were collected on indicated day post-transfer of engineered plasma cell populations (BCMs). FIG. 16B shows measurement of Factor IX activity in two studies. In a first study (bar graph), samples from mice receiving PBS only (grey bar) were compared to samples from mice treated with plasma cells engineered to express FIX (purple bar).

FIG. 17 shows hFIX activity of either recombinant FIX (BeneFIX®) or FIX isolated from plasma of mice NOG-hIL6 treated with plasma cells engineered to express FIX from an endogenous CCR5 locus. Activity level was measured for various indicated doses (total ug).

FIG. 18 shows average pg hDNA measured in various tissues collected at indicated time points from mice treated with plasma cells engineered for expression of FIX from an endogenous CCR5 locus.

FIG. 19 demonstrates FIX activity of two different measurements collected from murine models either at day 126 (A) or a pooled sample from days 98 and day 126 (B).

FIG. 20 demonstrates activated partial thromboplastin clotting time (aPPT) to quantify enzymatic activity of FIX isolated and purified from the supernatant of engineered B lineage cell populations in the presence of vitamin K (donors A-C) as compared to a control without vitamin K.

FIG. 21 shows human genomic DNA present in femur or spinal column bone marrow tissues of mice after treatment with plasma cell populations engineered for expression of FIX.

FIG. 22 demonstrates viability and percentage of an engineered B lineage cell preparation that is CD27 positive, CD38 positive, CD27 and CD38 positive, and Factor IX (F9) transgene insertion before (Time 0) and during undergoing the cryopreservation methods described herein.

FIG. 23 shows measurements of human Factor IX (huFIX) over time for mice after administration of B lineage cell population that were engineered using the methods and expression cassettes as disclosed herein.

FIGS. 24A-24B show measurements of human Factor IX (huFIX) and human immunoglobulin G (IgG) over time for mice after administration of four donors of B lineage cell populations (LKP23008, LKP22090, LKP22094, and LKP22091) that were engineered using the methods and expression cassettes as disclosed herein.

FIGS. 25A-25B show measurements of huFIX, IgG, and human immunoglobulin M (huIgM) over time for mice after administration and re-dosing at day 21 with engineered B lineage cell population using the methods disclosed herein.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Activation: As used herein, the term “activation” refers to the state of a cell, for example a B cell that has been sufficiently stimulated to induce detectable cellular proliferation or has been stimulated to exert its effector function. Activation can also be associated with induced cytokine production, cell signaling, differentiation, and/or antigen processing and presentation.

Administration: As used herein, the term “administration” typically refers to the administration (e.g., of a composition or treatment) to a subject or system (e.g., that is or comprises one or more cells, tissues, organisms, etc), for example to achieve delivery of an agent that is, is included in, or is otherwise delivered or generated by, such composition or treatment. Those of ordinary skill in the art will be aware of a variety of routes that may, in appropriate circumstances, be utilized for administration to a subject, for example a human. For example, in some embodiments, administration may be ocular, oral, parenteral, topical, etc., In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: As used herein, the term “agent” (or “biological agent” or “therapeutic agent”), refers to a molecule that may be expressed, released, secreted or delivered to a target by a modified cell described herein. An agent includes, but is not limited to, a nucleic acid, an antibiotic, an anti-inflammatory agent, an antibody or fragments thereof, an antibody agent or fragments thereof, a growth factor, a cytokine, an enzyme, a protein (e.g., an RNAse inhibitor), a peptide, a fusion protein, a synthetic molecule, an organic molecule (e.g., a small molecule), a carbohydrate, a lipid, a hormone, a microsome, a derivative or a variation thereof, and any combinations thereof. An agent may bind any cell moiety, such as a receptor, an antigenic determinant, or other binding site present on a target or target cell. An agent may diffuse or be transported into a cell, where it may act intracellularly.

Alloantigen: The term “alloantigen”, as used herein, refers to an antigen associated with allorecognition and/or graft rejection (e.g., an antigen against which a rejection immune response is directed). In general, alloantigens are agents that are present in or on tissue from one individual (e.g., a donor individual) of a particular species, but not in or on tissue from another individual (e.g., a recipient individual, for example who is genetically different from the donor individual) of the species, so that transfer of tissue from the donor individual to the recipient individual risks and/or results in a rejection immune response. In general, an antigen may be or include any chemical entity such as, for example, a small molecule, a nucleic acid, a polypeptide, a carbohydrate, a lipid, etc. In some embodiments, an alloantigen is or comprises a polypeptide. A variety of polypeptides are known in the art whose amino acid sequences can vary between and among individuals of the same species such that they might act as alloantigens.

Allogeneic: As used herein, the term “allogeneic” refers to any material (e.g., a population of cells) derived from a different animal of the same species.

Allorecognition: The term “allorecognition”, as used herein, typically refers to an immune response mounted by the immune system of an individual (i.e., a recipient) who receives a tissue graft from another individual (i.e., a donor, who for example is genetically distinct from the recipient individual) of the same species, which immune response involves recognition of an alloantigen on the grafted tissue. Typically, allorecognition involves T cell recognition of the alloantigen. In many embodiments, T cells recognize an alloantigen peptide, for example, encoded by a polymorphic gene whose sequence differs between the donor and recipient individuals.

Amelioration: As used herein, refers to the prevention, reduction or palliation of a state, or improvement of the state of a subject. Amelioration includes, but does not require complete recovery or complete prevention of a disease, disorder or condition (e.g., radiation injury).

Antigen: As used herein, the term “antigen” or “Ag” refers to a molecule that is capable of provoking an immune response. This immune response may involve either antibody production, activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequence or a partial nucleotide sequence encoding a protein that elicits an immune response encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

Antibody agent: As used herein, the term “antibody agent” (interchangeably referred to herein as “antibody”) refers to a polypeptide that may be expressed, released, secreted, or delivered to a target by a modified cell described herein. The polypeptide includes canonical immunoglobulin sequence elements sufficient to confer specific binding to a particular target antigen. In some embodiments, an antibody agent comprises of an antibody. As is known in the art, antibodies as produced in nature are approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains—an amino-terminal variable (VL) domain, followed by a carboxy-terminal constant (CL) domain, separated from one another by another “switch”. Intact antibody agent tetramers comprises two heavy chain-light chain dimers in which the heavy and light chains are linked to one another by a single disulfide bond; two other disulfide bonds connect the heavy chain hinge regions to one another, so that the dimers are connected to one another, and a tetramer is formed. Antibody agents are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally occurring antibodies binds to elements of the complement system, and also to receptors on effector cells, including, for example, effector cells that mediate cytotoxicity. Affinity and/or other binding attributes of Fc regions for Fc receptors can be modulated through glycosylation or other modification. In some embodiments, antibodies produced and/or utilized in accordance with the present disclosure (e.g., as a component of a CAR) include glycosylated Fc domains, including Fc domains with modified or engineered glycosylation. In some embodiments, any polypeptide or complex of polypeptides that includes sufficient immunoglobulin domain sequences as found in natural antibodies can be referred to and/or used as an “antibody agent”, whether such polypeptide is naturally produced (e.g., generated by an organism reacting to an antigen), or produced by recombinant engineering, chemical synthesis, or other artificial system or methodology. In some embodiments, an antibody agent is polyclonal. In some embodiments, an antibody agent is monoclonal. In some embodiments, an antibody agent has constant region sequences that are characteristic of mouse, rabbit, primate, or human antibodies. In some embodiments, antibody agent sequence elements are humanized, primatized, chimeric, etc, as is known in the art. Moreover, the term “antibody agent”, as used herein, can refer in appropriate embodiments (unless otherwise stated or clear from context) to any of the art-known or developed constructs or formats for utilizing antibody structural and functional features in alternative presentation. In some embodiments, an antibody agent may lack a covalent modification (e.g., attachment of a glycan) that it would have if produced naturally. In some embodiments, an antibody agent may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Autologous: As used herein, the term “autologous” refers to any material derived from an individual to which it is later to be re-introduced into the same individual.

Biologically active: As used herein, refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Biomarker: The term “biomarker” is used herein, consistent with its use in the art, to refer to a to an entity, event, or characteristic whose presence, level, degree, type, and/or form, correlates with a particular biological event or state of interest, so that it is considered to be a “marker” of that event or state. To give but a few examples, in some embodiments, a biomarker may be or comprise a marker for a particular disease state, or for likelihood that a particular disease, disorder or condition may develop, occur, or reoccur. In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. A biomarker may be or comprise an entity of any chemical class, and may be or comprise a combination of entities. For example, in some embodiments, a biomarker may be or comprise a nucleic acid, a polypeptide, a lipid, a carbohydrate, a small molecule, an inorganic agent (e.g., a metal or ion), or a combination thereof. In some embodiments, a biomarker is a cell surface marker. In some embodiments, a biomarker is intracellular. In some embodiments, a biomarker is detected outside of cells (e.g., is secreted or is otherwise generated or present outside of cells, e.g., in a body fluid such as blood, urine, tears, saliva, cerebrospinal fluid, etc. In some embodiments, a biomarker may be or comprise a genetic or epigenetic signature. In some embodiments, a biomarker may be or comprise a gene expression signature.

Bispecific antibody: As used herein, refers to a bispecific binding agent in which at least one, and typically both, of the binding moieties is or comprises an antibody component. A variety of different bi-specific antibody structures are known in the art. In some embodiments, each binding moiety in a bispecific antibody that is or comprises an antibody component includes VH and/or VL regions; in some such embodiments, such VH and/or VL regions are those found in a particular monoclonal antibody. In some embodiments, where the bispecific antibody contains two antibody component-binding moieties, each includes VH and/or VL regions from different monoclonal antibodies. In some embodiments, a bispecific antibody contains two antibody component binding moieties, wherein one of the two antibody component binding moieties includes an immunoglobulin molecule having VH and/or VL regions that contain CDRs from a first monoclonal antibody, and one of the two antibody component binding moieties includes an antibody fragment (e.g., Fab, F(ab′), F(ab′)2, Fd, Fv, dAB, scFv, etc.) having VH and/or VL regions that contain CDRs from a second monoclonal antibody.

Conservative sequence modifications: As used herein, the term “conservative sequence modifications” refers to amino acid modifications that do not significantly affect or alter the binding characteristics of an antibody containing the amino acid sequence. Such conservative modifications include amino acid substitutions, additions and deletions. Modifications can be introduced into an antibody compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

Encoding: As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in an engineered polynucleotide and/or when a particular residue in a polynucleotide is non-naturally occurring and/or is caused through action of the hand of man to be linked with an entity or moiety with which it is not linked in nature. For example, in some embodiments described and/or utilized herein, an engineered polynucleotide comprises a regulatory sequence that is found in nature in operative association with a first coding sequence but not in operative association with a second coding sequence, is linked by the hand of man so that it is operatively associated with a second coding sequence. Comparably, a polypeptide may be considered to be “engineered” if encoded by or expressed from an engineered polynucleotide, and/or if produced other than natural expression in a cell. Analogously, a cell or organism is considered to be “engineered” if it has been subjected to a manipulation, so that its genetic, epigenetic, and/or phenotypic identity is altered relative to an appropriate reference cell such as otherwise identical cell that has not been so manipulated. In some embodiments, such manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity.

Endogenous: As used herein “endogenous” refers to any material from or produced inside a particular organism, cell, tissue, or system.

Excipient: As used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Exogenous: As used herein, the term “exogenous” refers to any material introduced from or produced outside a particular organism, cell, tissue, or system.

Expand: As used herein, the term “expand” refers to increasing in number, as in an increase in the number of cells, for example, monocytes, macrophages, and/or dendritic cells. In one embodiment, monocytes, macrophages, or dendritic cells that are expanded ex vivo increase in number relative to the number originally present in a culture. In another embodiment, monocytes, macrophages, or dendritic cells that are expanded ex vivo increase in number relative to other cell types in a culture. In some embodiments, expansion may occur in vivo. The term “ex vivo,” as used herein, refers to cells that have been removed from a living organism, (e.g., a human) and propagated outside the organism (e.g., in a culture dish, test tube, or bioreactor).

Expression: As used herein, the term “expression” of a nucleic acid sequence refers to generation of any gene product from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Expression vector: As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses).

Fragment: As used herein, the terms “fragment” or “portion,” as used interchangeably herein, refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, a nucleotide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., nucleic acids) as found in the whole nucleotide. In some embodiments, a nucleotide fragment comprises or consists of at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more of the monomeric units (e.g., residues) found in the whole nucleotide. The whole material or entity may in some embodiments be referred to as the “parent” of the whole.

Functional: As used herein, a “functional” biological molecule is a biological molecule in a form in which it exhibits a property and/or activity by which it is characterized.

Gene product or expression product: As used herein, the term “gene product” or “expression product” generally refers to an RNA transcribed from a gene (pre- and/or post-processing) or a polypeptide (pre- and/or post-modification) encoded by an RNA transcribed from a gene.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences.

Identity: As used herein, the term “identity” refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

Immune cell: As used herein, the term “immune cell,” refers to a cell that is involved in an immune response, e.g., promotion of an immune response. Examples of immune cells include, but are not limited to, macrophages, monocytes, dendritic cells, neutrophils, eosinophils, mast cells, platelets, large granular lymphocytes, Langerhans' cells, natural killer (NK) cells, T-lymphocytes, plasma cells, plasmablasts, or B-lymphocytes. A source of immune cells (e.g., macrophages, monocytes, or dendritic cells) can be obtained from a subject.

Immune response: As used herein the term “immune response” refers to a cellular and/or systemic response to an antigen that occurs when lymphocytes identify antigenic molecules as foreign and induce the formation of antibodies and/or activate lymphocytes to remove the antigen.

Immunoglobulin: As used herein, the term “immunoglobulin” or “Ig,” refers to a class of proteins that function as antibodies. Antibodies expressed by B cells are sometimes referred to as a BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions, and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is an immunoglobulin that has no known antibody function but may serve as an antigen receptor. IgE is an immunoglobulin that mediates immediate hypersensitivity by causing release of mediators from mast cells and basophils upon exposure to allergen.

Isolated: As used herein, refers to a substance and/or entity that has been (1) separated from at least some of the components with which it was associated when initially produced (whether in nature and/or in an experimental setting) and/or otherwise previously associated, and/or (2) designed, produced, prepared, and/or manufactured by the hand of man. In some embodiments, a substance may be considered to be “isolated” if it is (or has been caused to be) free of or separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of other components (e.g., components with which it was previously associated). In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of a substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Marker: A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of presence or absence of a marker may vary depending upon a particular marker. In some embodiments, detection of a marker is highly specific in that it reflects a high probability that such tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. Those skilled in the art will appreciate that, in many embodiments, a useful marker need not distinguish with 100% accuracy.

Modified: As used herein, the term “modified” refers to a changed state or structure of a molecule or cell of the invention. Molecules may be modified in many ways, including chemically, structurally, and functionally. Cells may be modified through the introduction of nucleic acids.

Modulating: As used herein the term “modulating,” refers to mediating a detectable increase or decrease in the level of a response and/or a change in the nature of a response in a subject compared with the level and/or nature of a response in the subject in the absence of a treatment or compound, and/or compared with the level and/or nature of a response in an otherwise identical but untreated subject. The term encompasses perturbing and/or affecting a native signal or response thereby mediating a beneficial therapeutic response in a subject, preferably, a human.

Nucleic acid: As used herein, the term “nucleic acid” refers to a polymer of at least three nucleotides. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro), reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Operably linked: As used herein, the term “operably linked” refers to functional linkage between, for example, a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Payload: In general, the term “payload”, as used herein, refers to an agent that may be delivered or transported by association with another entity. In some embodiments, such association may be or include a covalent linkage; in some embodiments such association may be or include non-covalent interaction(s). In some embodiments, association may be direct; in some embodiments, association may be indirect. The term “payload” is not limited to a particular chemical identity or type; for example, in some embodiments, a payload may be or comprise, for example, an entity of any chemical class including, for example, a lipid, a metal, a nucleic acid (e.g., a transgene), a polypeptide, a saccharide (e.g., a polysaccharide), small molecule, or a combination or complex thereof. In some embodiments, a payload may be or comprise a biological modifier, a detectable agent (e.g., a dye, a fluorophore, a radiolabel, etc.), a detecting agent, a nutrient, a therapeutic agent, etc., or a combination thereof. In some embodiments, a payload may be or comprise a cell or organism, or a fraction, extract, or component thereof. In some embodiments, a payload may be or comprise a natural product in that it is found in and/or is obtained from nature; alternatively or additionally, in some embodiments, the term may be used to refer to one or more entities that is man-made in that it is designed, engineered, and/or produced through action of the hand of man and/or is not found in nature. In some embodiments, a payload may be or comprise an agent in isolated or pure form; in some embodiments, such agent may be in crude form.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Polynucleotide: As used herein, the term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; (in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: As used herein, a response to treatment may refer to a beneficial alteration in a subject's condition that occurs as a result of or correlates with treatment. In some embodiment, such alteration may be or comprise stabilization of a condition (e.g., prevention of deterioration that would have taken place in the absence of a treatment), amelioration of symptoms of a condition, and/or improvement in prospects for cure of a condition, etc. In some embodiments, the term “response” may refer to a response of a particular system or components thereof (e.g., of a particular cell, tissue, organism, or subject). Those skilled in the art will be aware of technologies available to assess a response of interest.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humor, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchoalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc. In some embodiments, a sample may be a “crude” sample in that it has been subjected to relatively little processing and/or is complex in that it includes components of relatively varied chemical classes.

Signal transduction pathway: As used herein, the term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

Significant: As used herein, the term “significant” typically refers to the context wherein the difference or relationship between two variables (e.g., sequence identity, protein production, spatiotemporal conditions, etc.) are certain and exist. Significance can be statistically measured (e.g., statistically significant) by various mathematical formulas and models as understood by one skilled in the art. These methods include, but are not limited to, student t-test, two-tailed test, analysis of variance (ANOVA), etc. Furthermore, significance can impart differences within structure and chemistry between two different entities. For example, a sample molecule may be compared to a reference molecule and exhibits a structurally difference from said reference molecule that is significant, e.g., in the presence or absence or in the level of one or more biological or chemical moieties as compared to the reference entity.

Source: The term “source” as used herein, typically refers to a context in which an agent of interest (e.g., that may be or comprise a carbohydrate, a lipid, a nucleic acid, a metal, polypeptide, a small molecule, or a combination thereof) may be found in nature, or from which such agent can be or has been obtained (e.g., isolated). In some embodiments, a source may be or comprise a biological source (e.g., an organism, tissue, or cell, or sample thereof); in some embodiments, a source may be an environmental source. In some embodiments, a source may be or comprise a primary sample from an organism (e.g., which may be or comprise a tissue or fluid of such organism, and/or may be or comprise cell(s) of such organism). In some embodiments, an organism may be or comprise a prokaryotic organism (e.g., a bacterium) or a eukaryotic organism (e.g., a fungus or yeast, an insect, a mammal, a plant, a reptile, etc.). In some embodiments, an infectious agent such as a virus or phage may be considered an organism for purposes of this disclosure, and in particular with respect to being a source. In some embodiments, a source may be or comprise an engineered source, such as a cell line or culture, an in vitro system, etc.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments a human subject is an adult, adolescent, or pediatric subject. In some embodiments, a subject is suffering from a disease, disorder or condition, e.g., a disease, disorder, or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Substantial identity: As used herein, the term “substantial identity” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having an amino acid sequence at least 80%, preferably at least 85%, at least 90%, at least 95%, at least 98% or at least 99% identical to that of a reference CDR.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Substantially purified: As used herein, the term “substantially purified”, for example as applied to a cell, refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

Target: As used herein, the term “target” refers to a cell, tissue, organ, or site within the body that is the subject of provided methods, systems, and/or compositions, for example, a cell, tissue, organ or site within a body that is in need of treatment or is preferentially bound by.

Target Locus: As used herein, the term “target locus” may refer to a specific site or location on a chromosome of interest. For example, a target locus may be a site to be “engineered” or “altered” by the hand of man. In some embodiments described and/or utilized herein, an engineered polynucleotide comprises homology to a target locus in order to allow further alterations at a specific site (e.g., CCR5 as a target locus, whose homologous sequence may be part of a guide RNA to result in incorporation of an edit via CRISPR/Cas-mediated gene editing). In some embodiments, target locus may interchangeably refer to a target gene of interest for manipulation by man. In some embodiments, such target locus manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols).

Target site: As used herein, the term “target site” or “target sequence” refers to a genomic nucleic acid sequence that defines a portion of a nucleic acid to which a binding molecule may specifically bind under conditions sufficient for binding to occur.

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Transfected: As used herein, the term “transfected” or “transformed” or “transduced” refers to a process by which exogenous nucleic acid is transferred or introduced into the host cell. A “transfected” or “transformed” or “transduced” cell is one which has been transfected, transformed, or transduced with exogenous nucleic acid. The cell includes the primary subject cell and its progeny.

Treat: As used herein, the term “treat,” “treatment,” or “treating” refers to partial or complete alleviation, amelioration, delay of onset of, inhibition, prevention, relief, and/or reduction in incidence and/or severity of one or more symptoms or features of a disease, disorder, and/or condition. In some embodiments, treatment may be administered to a subject who does not exhibit signs or features of a disease, disorder, and/or condition (e.g., may be prophylactic). In some embodiments, treatment may be administered to a subject who exhibits only early or mild signs or features of the disease, disorder, and/or condition, for example for the purpose of decreasing the risk of developing pathology associated with the disease, disorder, and/or condition. In some embodiments, treatment may be administered to a subject who exhibits established, severe, and/or late-stage signs of the disease, disorder, or condition.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more biological or chemical moieties as compared to the reference entity. In some embodiments, a variant also differs functionally from its reference molecule. In some embodiments, a variant differs structurally but performs the same or similar function as its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to one another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a variant polypeptide or nucleic acid is a truncated form of the reference polypeptide or nucleic acid. In some embodiments, a variant polypeptide that is a truncated form of the reference polypeptide may demonstrate comparable, identical, or greater levels of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions.

Vector: As used herein, the term “vector” refers to a composition of matter that comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

The present disclosure encompasses culturing and engineering of B lineage cell populations in order to express FIX.

### Hemophilia B

Hemophilia is an X-linked recessive bleeding disorder caused by deficiencies of either coagulation factor VIII (FVIII) or factor IX (FIX), which are named hemophilia A and hemophilia B, respectively. Hemophilia B, when compared with hemophilia A, is much less common and is estimated to account for 15-20% of all hemophilia cases and is prevalent in about 1 in 30,000 male live births (Sidonio et al, 2021 and Shen et al, 2022). Progression of hemophilia B can propagate in severe problems with excessive bleeding, joint damage, and pain.

Over 1000 mutations in FIX have been identified in the pathogenesis of hemophilia B. These FIX mutations lead to FIX deficiency over multiple levels including its gene structure, gene transcription, splicing, translation, post-translation modifications, protein folding, and formation of a functional complex (Shen et al, 2022). Factor IX is a vitamin K-dependent serine protease that is essential for generation of thrombin, a key enzyme that causes clotting of the blood via the conversion of fibrinogen to fibrin. Before being secreted into the blood, FIX undergoes a number of intracellular processing, including cleavage and removal of the signal peptide and propeptide sequence.

Due to phenotypic heterogeneity among subjects with FIX mutations that result in hemophilia B, treatments have been sparse and inefficient. Current treatments include frequent intravenous administration of clotting factor concentrates to subjects or a liver-mediated gene therapy (HEMGENIX®) that results in numerous side effects such as elevated liver enzyme levels and flu-like symptoms. The present disclosure provides methods and preparations of engineered B lineage cell populations that may be useful as a viable cell therapy option for treatment of hemophilia generally, and specifically for hemophilia B. In some embodiments, engineered B lineage cell populations prepared via methods disclosed herein may express one or more therapeutic proteins, including, e.g., FIX.

### Cell Therapy

Cell-based therapeutics (cell therapies) are an emerging class of medicine that make use of innate cellular machinery to combat disease. Unlike many traditional treatment methods, cell therapies make use of cellular localization, migration, and proliferation within the body, which can translate to improved biodistribution and targeted delivery of therapeutics. Cell therapies also benefit from cellular ability to sense and respond to various extrinsic signals within a subject, including, e.g., small molecules, other cells, physical forces, and/or marker proteins. Cellular persistence in vivo also enables cell therapies to survive, differentiate, function, etc. within a subject over extended time periods. These innate qualities may also lead to improved safety and efficacy for cell therapies as compared to other biologics or pharmaceutical compounds, providing long-lived, specifically targeted, adjustable, and/or responsive treatment for disease.

Cell-based therapies may have potential applications for a broad range of diseases, including those that have proved intractable or difficult to manage with traditional treatment options. Diseases that have been targeted for cell-based treatment include, e.g., various cancers, autoimmune diseases, central nervous system (CNS) diseases, neurodegenerative disorders, and cardiovascular diseases, among others. Cellular therapeutics offer an alternative over other treatment options for diseases where highly specific targeting (e.g., to a particular tissue type, area of the body, etc.) and/or longer-term treatment efficacy (e.g., enabling lower dosage frequency, single treatment options, etc.) are highly favored or necessary.

Cell therapies may employ a number of different cell types, which are typically modified to provide a therapeutic effect (e.g., transgene expression, reprogrammed cellular targeting, etc.). Although many cell types have potential to provide some form of therapeutic effect, recent therapies have heavily favored adaptive immune cells such as T lymphocytes and B lymphocytes (referred to interchangeably herein as T cells and B cells, respectively). For example, chimeric antigen receptor T (CAR-T) cells have been engineered to treat various cancers through recognition of one or more tumor cell markers, leading to cytotoxic destruction of tumor cells. CAR-T cells are also being adapted to treat infectious diseases (e.g., HIV) through recognition of other targeted antigens. Recent engineering efforts are focused on enhancing CAR-T receptor functionality, reducing innate immune response to CAR-T, and developing allogeneic therapies that make use of donor cells.

### B Lineage Cell Therapies

While cell-based therapies present an exciting new avenue for treatment of diseases, there are numerous challenges including achievement of safe, specific, and long-term therapeutic changes within targeted cells or tissues while reducing off-target effects. Furthermore, immune tolerance of these cell-based therapies is essential in order to obviate any deleterious side effects (See Jeske et al. 2021, incorporated by reference herein in its entirety). In order to address these challenges, engineering of B lineage cells has also been an area of development for cell-based therapies, due to the natural role of B lineage cells in antibody production within the body while minimizing inflammation. Antibody-based treatments are an established, well-studied form of treatment for a number of diseases, including cancer, autoimmune diseases, and infectious diseases. Monoclonal antibodies can be produced to target antigens within the body, which is valuable for treatment of diseases where such antibodies cannot be induced through natural processes (e.g., self-antigens for cancer and/or autoimmune diseases, antigens that fail to elicit natural immune response through infection and/or vaccination, etc.). Current antibody therapies require frequent administration and are costly to produce. Researchers have attempted to address these issues through use of gene therapy, which makes use of various techniques (e.g., viral vectors, CRISPR/Cas9 editing) to deliver antibody payloads to endogenous cells in the body, leading to persistent antibody production within a subject. However, these approaches can result in low levels of antibody expression and a counter-active response by the subject's own immune system.

Furthermore, due to their minimal impact on a subject's native immune system and capacity to continually produce antibodies, B lineage cells are a highly desirable cell-based target to secrete other payloads including, but not limited to, enzymes, complement proteins, cytokines, cytokine receptors, chimeric antigen receptors (CARs), anti-fibrotic molecules, anti-thrombotic molecules, antigens, both wild type and variant proteins, coagulation factors, glucose response elements, and fragments of antibodies, antigens, and proteins. Recent reports have described successful engineering of B lineage cells, for example, producing human B-cell activating factor (hBAFF) in a murine model. Excitingly, this work not only demonstrates successful production of hBAFF, but also engraftment in the bone marrow and viability of these engineered B lineage cells up to sixty days post-engraftment (See, Cheng et al. 2022, incorporated herein by reference in its entirety). B lineage cells are an attractive option for development of cell-based therapies and may potentially offer improved therapeutic effects (e.g., payload delivery, targeting, long-term payload expression, reduced auto-immune response, etc.) as compared to traditional therapies (e.g., antibody-based therapeutics, other cell therapies, etc.).

In some embodiments, a B lineage cell is a cell that expresses one or more B cell receptors (BCR) on a cell membrane. In some embodiments, a B lineage cell is a modified version or variant of a cell that expresses one or more B cell receptors (BCRs) on a cell membrane. In some embodiments, a B lineage cell is a naïve or memory B cell. In some embodiments, a B lineage cell is a cell derived from a naïve B cell (e.g., activated B lineage cell, plasmablast, plasma cell) or a variant thereof. In some embodiments, a B lineage cell is an activated B lineage cell. In some embodiments, a B lineage cell is a plasmablast. In some embodiments, a B lineage cell is a plasma cell.

In some embodiments, a B lineage cell population comprise naïve B cells. In some embodiments, naïve B cell populations are used as reference cell populations. In some embodiments, naïve B cell populations express CD19 (CD19+). In some embodiments, expression of CD19 in naïve B cell populations is used as a reference to assist in characterization of other B lineage cell populations. In some embodiments, naïve B cell populations express CD20 (CD20+). In some embodiments, expression of CD20 in naïve B cell populations is used as a reference to assist in characterization of other B lineage cell populations. In some embodiments, naïve B cell populations express low amounts of CD27 (CD271°). In some embodiments, expression of CD27 in naïve B cell populations are as a reference to assist in characterization of other B lineage cell populations. In some embodiments, naïve B cell populations express low amounts of CD38 (CD381°). In some embodiments, expression of CD38 in naïve B cell populations are used as a reference to assist in characterization of other B lineage cell populations. In some embodiments, naïve B cell populations express low amounts of CD138 (CD138^{lo}). In some embodiments, expression of CD138 in naïve B cell populations are used as a reference to assist in characterization of other B lincage cell population.

In some embodiments, a B lineage cell population comprises activated B lineage cells. In some embodiments, activated B lineage cell populations are used as reference cell populations. In some embodiments, activated B lineage cell populations are compared to a reference cell population (e.g., naïve B cell population). In some embodiments, activated B lineage cell populations express lower amounts of CD19 (CD19^{lo}) as compared to a reference cell population (e.g., naïve B cell populations). In some embodiments, activated B lineage cell populations express different (e.g., higher or lower) amounts of CD19 as compared to a reference cell population (e.g., differentiated B cell populations, etc.). In some embodiments, activated B lineage cell populations express lower amounts of CD20 (CD20^{lo}) as compared to a reference cell population (e.g., naïve B cell populations). In some embodiments, activated B lineage cell populations express different (e.g., higher or lower) amounts of CD20 as compared to a reference cell population (e.g., differentiated B cell populations, etc.). In some embodiments, activated B lineage cell populations express higher amounts of CD27 (CD27^{hi}) as compared to a reference cell population (e.g., naïve B cell populations). In some embodiments, activated B lineage cell populations express different (e.g., higher or lower) amounts of CD27 as compared to a reference cell population (e.g., differentiated B cell populations, etc.). In some embodiments, activated B lineage cell populations express lower amounts of CD38 (CD381^{lo}) as compared to a reference cell population (e.g., naïve B cell populations). In some embodiments, activated B lineage cell populations express different (e.g., higher or lower) amounts of CD38 as compared to a reference cell population (e.g., differentiated B cell populations, etc.). In some embodiments, activated B lineage cell populations express lower amounts of CD138 (CD138^{lo}) as compared to a reference cell population (e.g., naïve B cell populations). In some embodiments, activated B lineage cell populations express different (e.g., higher or lower) amounts of CD138 as compared to a reference cell population (e.g., differentiated B cell populations, etc.).

In some embodiments, a B lineage cell population may comprise plasmablast cells. In some embodiments, plasmablast cell populations are used as reference cell populations. In some embodiments, plasmablast cell populations are compared to a reference cell population (e.g., naïve B cell population). In some embodiments, plasmablast cell populations express lower amounts of CD19 (CD19^{lo}) as compared to a reference cell population (e.g., naïve B cell populations). In some embodiments, plasmablast cell populations express different (e.g., higher or lower) amounts of CD19 as compared to a reference cell population (e.g., activated cell populations, plasma cell populations, etc.). In some embodiments, plasmablast cell populations express lower amounts of CD20 (CD20^{lo}) as compared to a reference cell population (e.g., naïve B cell populations). In some embodiments, plasmablast cell populations express different (e.g., higher or lower) amounts of CD20 as compared to a reference cell population (e.g., activated cell populations, plasma cell populations, etc.). In some embodiments, plasmablast cell populations express higher amounts of CD27 (CD27^{hi}) as compared to a reference cell population (e.g., naïve B cell populations). In some embodiments, plasmablast cell populations express different (e.g., higher or lower) amounts of CD27 as compared to a reference cell population (e.g., activated cell populations, plasma cell populations, etc.). In some embodiments, plasmablast cell populations express higher amounts of CD38 (CD38^{hi}) as compared to a reference cell population (e.g., naïve B cell populations). In some embodiments, plasmablast cell populations express different (e.g., higher or lower) amounts of CD38 as compared to a reference cell population (e.g., activated cell populations, plasma cell populations, etc.). In some embodiments, plasmablast cell populations express lower amounts of CD138 (CD138^{lo}) as compared to a reference cell population (e.g., naïve B cell populations). In some embodiments, plasmablast cell populations express different (e.g., higher or lower) amounts of CD138 as compared to a reference cell population (e.g., activated cell populations, plasma cell populations, etc.).

In some embodiments, a B lineage cell population comprises plasma cells. In some embodiments, plasma cell populations are used as reference cell populations. In some embodiments, plasma cell populations are compared to a reference cell population (e.g., naïve B cell population). In some embodiments, plasma cell populations express lower amounts of CD19 (CD19^{lo}) as compared to a reference cell population (e.g., naïve B cell populations). In some embodiments, plasma cell populations express different (e.g., higher or lower) amounts of CD19 as compared to a reference cell population (e.g., activated cell populations, plasmablast populations, etc.). In some embodiments, plasma cell populations express lower amounts of CD20 (CD20^{lo}) as compared to a reference cell population (e.g., naïve B cell populations). In some embodiments, plasma cell populations express different (e.g., higher or lower) amounts of CD20 as compared to a reference cell population (e.g., activated cell populations, plasmablast populations, etc.). In some embodiments, plasma cell populations express higher amounts of CD27 (CD27^{hi}) as compared to a reference cell population (e.g., naïve B cell populations). In some embodiments, plasma cell populations express different (e.g., higher or lower) amounts of CD27 as compared to a reference cell population (e.g., activated cell populations, plasmablast populations, etc.). In some embodiments, plasma cell populations express higher amounts of CD38 (CD38^{hi}) as compared to a reference cell population (e.g., naïve B cell populations). In some embodiments, plasma cell populations express different (e.g., higher or lower) amounts of CD38 as compared to a reference cell population (e.g., activated cell populations, plasmablast populations, etc.). In some embodiments, plasma cell populations express higher amounts of CD138 (CD138^{hi}) as compared to a reference cell population (e.g., naïve B cell populations). In some embodiments, plasma cell populations express different (e.g., higher or lower) amounts of CD138 as compared to a reference cell population (e.g., activated cell populations, plasmablast populations, etc.).

### Cell Engineering

Production of engineered cells for various uses including, e.g., cell therapies is an active area of development. Genomic and epigenomic modifications, synthetic biology, and application of biomaterials may be employed to generate engineered cells with desired properties for therapeutic applications. Selection of an appropriate method for generating engineered cells is typically dependent upon the desired output and effects of cell therapy and requires optimization for different cell types, transgenes of interest, etc. It is understood in the art that engineering methods that are effective for a particular cell type may be less effective (or not viable) for another cell type. Furthermore, engineering methods may vary depending upon whether a therapy requires cellular localization, expression of an endogenous or exogenous protein, removal of an endogenous protein, etc.

### Genome Editing

Genome editing tools can alter a cellular genome to produce a desired therapeutic effect, e.g., expression of a therapeutic protein. Targeted nucleases (e.g., Cas proteins, TALENS, ZFNs, etc.), viral vectors (e.g., AAV, lentiviral, adenoviral, etc.), recombinases (e.g., Cre recombinasc, Flp recombinase, PhiC31 integrasc, etc.), and other tools may be employed to provide genetic modifications. Gene editing efficiency may vary depending on, e.g., cell type, desired function, and ease of delivery. Accordingly, editing methods often require extensive optimization to provide engineered cells with intended functionality for therapeutic applications.

### B Lineage Cell Engineering

Various B lineage cell engineering techniques are described in the art, including, e.g., CRISPR/Cas9, AAV, lentiviral, and recombinase-based methods. These methods are generally employed to introduce a payload (e.g., expression cassette, transgene, etc.) into naïve B cells in order to express a protein of interest. A payload may be designed for episomal expression, integration into a specific target locus (e.g., endogenous target gene locus), or integration into a non-specific locus (e.g., endogenous random or non-target gene locus). Payloads may be designed such that an endogenous target gene locus continues to produce functional protein and/or fulfill its natural function (non-disruptive integration). Payloads may also be designed to intentionally disrupt an endogenous target gene locus to produce lowered or non-detectable levels of functional protein and/or some amount of non-functional protein.

Methods for integration of a payload (e.g., expression cassette comprising one or more transgenes) into an endogenous target locus may comprise site-specific cleavage with a targeted nuclease (e.g., Cas protein, including Cas9), followed by integration of a transgene (e.g., FIX) through an endogenous repair pathway (e.g., homologous recombination, homology-directed repair, etc.). In some embodiments, methods for integration of an expression cassette comprising a FIX transgene comprise site-specific cleavage at a target locus (e.g., CCR5) with a guide RNA/Cas9 complex, followed by integration of FIX at the target locus through homologous recombination.

In some embodiments, a method of B lineage cell engineering is or comprises administration of an ribonucleoprotein (RNP) to a cell population. In some embodiments, a method of B lineage cell engineering is or comprises administration of a composition comprising a Cas protein complexed with guide RNA (gRNA) to a cell population. In some embodiments, a method of B lineage cell engineering is or comprises administration of a composition comprising a Cas9/guide RNA complex to a cell population. In some embodiments, a method of B lineage cell engineering is or comprises administration of a composition comprising a Cas9/guide RNA complex to a cell population. In some embodiments, a method of B lineage cell engineering is or comprises administration of a composition comprising a payload (e.g., expression cassette comprising one or more transgenes) of interest to a cell population. In some embodiments, a method of B lineage cell engineering is or comprises administration of a composition comprising a payload (e.g., expression cassette comprising one or more transgenes) to a cell population through use of a viral vector. In some embodiments, a method of B lineage cell engineering is or comprises administration of a composition comprising a payload (e.g., expression cassette comprising one or more transgenes) encapsulated within an AAV capsid (e.g., AAV2, AAV3, AAV5, AAV6, AAV8, etc.) to a cell population. In some embodiments, a method of B lineage cell engineering is or comprises administration of a composition comprising a transgene encapsulated within an AAV capsid (e.g., AAV2, AAV3, AAV5, AAV6, AAV8, etc.) in combination with or in addition to administration of a composition comprising a Cas9/gRNA complex.

In some embodiments, a method of B lineage cell engineering comprises a step of electroporation to facilitate cellular uptake of one or more engineering components. In some embodiments, a method of B lineage cell engineering comprises a step of electroporation to facilitate cellular uptake of a Cas9/gRNA complex. In some embodiments, a method of B lineage cell engineering comprises a step of electroporation to facilitate cellular uptake of a Cas9/gRNA complex and a payload. In some embodiments, a method of B lineage cell engineering may comprise a step of electroporation to facilitate cellular uptake of a Cas9/gRNA complex and a payload (e.g., expression cassette comprising one or more transgenes) encapsulated in an AAV capsid (AAV2, AAV3, AAV5, AAV6, AAV8, etc.). In some embodiments, a method of B lineage cell engineering comprises a step of electroporation to facilitate cellular uptake of a Cas9/gRNA complex and a payload (e.g., expression cassette comprising one or more transgenes) encapsulated in an AAV6 capsid.

In some embodiments, a method of B lineage cell engineering comprises a step of viral transduction to facilitate cellular uptake of a payload (e.g., expression cassette comprising one or more transgenes). In some embodiments, a method of B lineage cell engineering comprises a step of viral transduction to facilitate cellular uptake of a payload (e.g., transgene) encapsulated in an AAV capsid (AAV2, AAV3, AAV5, AAV6, AAV8, ctc.). In some embodiments, a method of B lineage cell engineering comprises a step of viral transduction to facilitate cellular uptake of a payload (e.g., expression cassette comprising one or more transgenes) encapsulated in an AAV6 capsid. In some embodiments, a method of B lineage cell engineering comprises one or more steps of: (i) electroporation to facilitate cellular uptake of a Cas9/gRNA complex; and (ii) viral transduction to facilitate cellular uptake of a payload encapsulated in an AAV capsid.

In some embodiments, a method of cell engineering is particularly effective for one type of cell (e.g., T cell) and less effective for another type of cell (e.g., B cell). In some embodiments, a method of B lineage cell engineering provides improved genome editing efficiency in B lineage cells as compared to other cell types (e.g., T cell). In some embodiments, a method of B cell engineering provides at least about 10%, 15%, 20%, 25%, 30%, 35%, 40%, 45%, 50%, 55%, 60%, 65%, 70%, 75%, 80%, 85%, 90%, 95%, or 99% editing efficiency. In some embodiments, a method of B lineage cell engineering provides at least about 1E6, 1.5E6, 2E6, 2.5E6, 3E6, 3.5E6, 4E6, 4.5E6, 5E6, 5.5E6, 6E6, 6.5E6, 7E6, 7.5E6, 8E6, 8.5E6, 9E6, 9.5E6, 1E7, 1.5E7, 2E7, 2.5E7, 3E7, 3.5E7, 4E7, 4.5E7, or 5E7 edited B lineage cells.

In some embodiments, a method of B lineage cell engineering comprises a step of editing activated B cells. In some embodiments, a method of B lineage cell engineering comprises a step of editing B lineage cells after an activation step of about 1, 2, 3, 4, or 5 day(s). In some embodiments, a method of B lineage cell engineering comprises a step of editing B lineage cells after an activation step of 2 days. In some embodiments, a method of B lineage cell engineering comprises a step of editing B lineage cells after an activation step of about 1, 2, 3, 4, or 5 day(s) and expanding edited B lineage cells in the activation media for an additional period of about 1, 2, 3, 4, 5, 6, 7, or 8 days. In some embodiments, a method of B lineage cell engineering comprises a step of editing B lineage cells after an activation step of 2 days and expanding edited B lineage cells in the activation media for an additional 6 days.

### Payloads

Various methods described herein may be used for generation of engineered cells (e.g., B lincage cell populations, etc.) comprising one or more payloads. In some embodiments, engineered cells (e.g., B lineage cell populations, etc.) comprise a polynucleotide sequence encoding one or more payloads. In accordance with various aspects of the present disclosure, any of a variety of payloads may be used (e.g., those with a therapeutic or monitoring purpose), alone or in combination. In some embodiments, a payload is or comprises a polynucleotide sequence encoding a peptide or polypeptide. In some embodiments, a payload is or comprises one or more transgenes. In some embodiments, a payload is or comprises one or more homology arm sequences. In some embodiments, a payload is or comprises a transgene flanked by one or more homology sequences.

In some embodiments, a payload may be or comprise a polynucleotide sequence, which comprises an expression cassette. In some embodiments, an expression cassette comprises one or more polynucleotide sequence elements (e.g., promoters, enhancers, transgenes, termination elements, homology arms, biomarkers, signal peptide sequences, internal ribosome entry site elements, self-cleaving peptide sequences, ubiquitous chromatin opening element, etc.). In some embodiments, an expression cassette comprises one or more polynucleotide sequence elements (e.g., promoters, enhancers, transgenes, termination elements, homology arms, biomarkers, signal peptide sequences, internal ribosome entry site elements, self-cleaving peptide sequences, ubiquitous chromatin opening element, etc.) in a particular configuration and/or combination. In some embodiments, an expression cassette comprises one or more polynucleotide sequence elements (e.g., promoters, enhancers, transgenes, termination elements, homology arms, biomarkers, signal peptide sequences, internal ribosome entry site elements, self-cleaving peptide sequences, ubiquitous chromatin opening element, etc.) in a particular configuration and/or combination in order to promote expression of a transgene in a cell population. In some embodiments, an expression cassette comprises one or more polynucleotide sequence elements (e.g., promoters, enhancers, transgenes, termination elements, homology arms, biomarkers, signal peptide sequences, internal ribosome entry site elements, self-cleaving peptide sequences, ubiquitous chromatin opening element, etc.) in a particular configuration and/or combination in order to promote expression of a transgene in an engineered B lineage cell population.

In some embodiments, an expression cassette comprises one or more polynucleotide sequences encoding one or more promoters (e.g., MND, CMV, FEEK I, SFFV, PGK, EF-1a, B2M, etc.). In some embodiments, an expression cassette comprises one or more polynucleotide sequences encoding one or more exogenous promoters. In some embodiments, an expression cassette comprises one or more polynucleotide sequences encoding one or more endogenous promoters. In some embodiments, an expression cassette does not comprise one or more promoters. In some embodiments, an expression cassette does not comprise one or more exogenous promoters. In some embodiments, an expression cassette does not comprise one or more endogenous promoters.

In some embodiments, an expression cassette comprises one or more polynucleotide sequences encoding a translation initiation site (e.g., Kozak consensus sequence, ribosomal binding site, etc.). In some embodiments, an expression cassette comprises one or more exogenous polynucleotide sequences encoding a translation initiation site. In some embodiments, an expression cassette comprises one or more endogenous polynucleotide sequences encoding a translation initiation site. In some embodiments, an expression cassette does not comprise one or more polynucleotide sequences encoding a translation initiation site. In some embodiments, an expression cassette does not comprise one or more exogenous polynucleotide sequences encoding a translation initiation site. In some embodiments, an expression cassette does not comprise one or more endogenous polynucleotide sequences encoding a translation initiation site.

In some embodiments, an expression cassette comprises one or more polynucleotide sequences encoding one or more enhancers (e.g., WPRE, beta-globin, etc.). In some embodiments, an expression cassette comprises one or more polynucleotide sequences encoding one or more exogenous enhancers. In some embodiments, an expression cassette comprises one or more polynucleotide sequences encoding one or more endogenous enhancers. In some embodiments, an enhancer may be viral (e.g., WPRE, etc.) or non-viral. In some embodiments, an expression cassette does not comprise one or more enhancers. In some embodiments, an expression cassette does not comprise one or more exogenous enhancers. In some embodiments, an expression cassette does not comprise one or more endogenous enhancers.

In some embodiments, an expression cassette comprises one or more polynucleotide sequences encoding one or more terminators (e.g., polyA, including, e.g., BGH polyA, SV40 polyA, etc.). In some embodiments, an expression cassette comprises one or more polynucleotide sequences encoding one or more exogenous terminators. In some embodiments, an expression cassette comprises one or more polynucleotide sequences encoding one or more endogenous terminators. In some embodiments, an expression cassette does not comprise one or more terminators. In some embodiments, an expression cassette does not comprise one or more exogenous terminators. In some embodiments, an expression cassette does not comprise one or more endogenous terminators.

In some embodiments, an expression cassette comprises a polynucleotide sequence encoding a transgene (e.g., FIX) or variant thereof. In some embodiments, an expression cassette comprises a polynucleotide sequence encoding FIX or a variant thereof (e.g., FIX R338L). In some embodiments, an expression cassette comprises a polynucleotide sequence encoding a transgene (e.g., FIX) or variant thereof for expression of a peptide or polypeptide (e.g., FIX) or variant thereof.

### Transgenes

In some embodiments, a transgene is a corrective gene chosen to improve one or more signs and/or symptoms of a disease, disorder, or condition. In some embodiments, transgenes are functional versions of disease associated genes (i.e., gene isoform(s) which are associated with the manifestation or worsening of a disease, disorder, or condition) found in a subject. In some embodiments, one or more transgenes are optimized versions of disease-associated genes found in a subject (e.g., codon optimized or expression-optimized variants). In some embodiments, transgenes are variants of disease-associated genes found in a subject (e.g., a functional gene fragment or variant thereof). In some embodiments, a transgene is a gene that causes expression of a peptide that is normally expressed in one or more healthy tissues.

In some embodiments, a transgene is a gene that causes expression of an altered protein with a gain- or loss-of-function mutation. In some embodiments, a transgene is a gene that causes expression of a fusion protein. In some embodiments, a transgene is a gene that causes expression of an antibody agent. In some embodiments, a transgene is a gene that causes expression of a multi-specific antibody. In some embodiments, a transgene is a fragment of an antibody, antigen, or protein. In some embodiments, a transgene is a gene that causes expression an enzyme (e.g., for enzyme replacement therapy). In some embodiments, a transgene is a gene that causes expression of a cytokine. In some embodiments, a transgene is a gene that causes expression of a cytokine receptor. In some embodiments, a transgene is a gene that causes expression of a chimeric antigen receptor (CAR). In some embodiments, a transgene is a gene that causes expression of an anti-thrombotic molecule. In some embodiments, a transgene is a gene that causes expression of a coagulation factor. In some embodiments, a transgene is a gene that causes expression of a glucose response element. In some embodiments, a transgene is a gene that causes expression of a nanobody. In some embodiments, a transgene is a gene that causes expression of Factor IX (FIX) or a variant thereof.

In some embodiments, a transgene is or comprises a gene encoding a functional nucleic acid. In some embodiments, a therapeutic agent is or comprises an agent that has a therapeutic effect upon a host cell or subject (including, e.g., a ribozyme, guide RNA (gRNA), antisense oligonucleotide (ASO), miRNA, siRNA, and/or shRNA). For example, in some embodiments, a therapeutic agent promotes a biological process to treat a medical condition, e.g., at least one symptom of a disease, disorder, or condition such as Hemophilia B.

In some embodiments, transgene expression in a subject result substantially from integration at a target locus. In some embodiments, 75% or more (e.g., 80% or more, 85% or more, 90% or more, 95% or more, 99% or more, 99.5% or more) of total transgene expression in a subject is from transgene integration at a target locus. In some embodiments, 25% or less (e.g., 20% or less, 15% or less, 10% or less, 5% or less, 1% or less, 0.5% or less, 0.1% or less) of total transgene expression in a subject is from a source other than transgene integration at a target locus (e.g., episomal expression, integration at a non-target locus).

In some embodiments, a transgene is or comprises a sequence having at least 80%, 85%, 90%, 95%, 99%, or 100% identity to a corresponding wild-type reference nucleotide sequence (e.g., a wild-type gene sequence). In some embodiments, a transgene is or comprises a sequence having a least 80%, 85%, 90%, 95%, 99%, or 100% identity to a portion of a corresponding wild-type reference nucleotide sequence (e.g., a wild-type gene sequence).

### Homology Arms

In some embodiments, a payload (e.g., expression cassette) may comprise one or more flanking polynucleotide sequences with significant sequence homology to a target locus (e.g., homology arms). In some embodiments, homology arms flank a polynucleotide sequence encoding a payload (e.g., one homology arm is 5′ to a payload (also referred to herein as a 5′ homology arm) and one homology arm is 3′ to a payload (also referred to herein as a 3′ homology arm)). In some embodiments, homology arms direct site-specific integration of a payload.

In some embodiments, homology arms are between 400 and 1000 nt in length. In some embodiments, homology arms are between 800 and 1500 nt in length. In some embodiments, homology arms are at least 500 nt in length. In some embodiments, homology arms are less than 1500 nt in length. In some embodiments, homology arms are 1000 nt in length. In some embodiments, homology arms are at least 50 nt in length. In some embodiments, homology arms are at least 600 nt in length. In some embodiments, homology arms are of the same length. In some embodiments, homology are not of the same length. In some embodiments, homology arms have at least 70% sequence homology to a target locus. In some embodiments, homology arms have at least 80% sequence homology to a target locus. In some embodiments, homology arms have at least 90% sequence homology to a target locus. In some embodiments, homology arms have at least 95% sequence homology to a target locus. In some embodiments, homology arms have at least 99% sequence homology to a target locus. In some embodiments, homology arms have 100% sequence homology to a target locus. In some embodiments, homology arms have at least 70% sequence identity to a target locus. In some embodiments, homology arms have at least 80% sequence identity to a target locus. In some embodiments, homology arms have at least 90% sequence identity to a target locus. In some embodiments, homology arms have at least 95% sequence identity to a target locus. In some embodiments, homology arms have at least 99% sequence identity to a target locus. In some embodiments, homology arms have 100% sequence identity to a target locus.

In some embodiments, constructs comprising homology arms provide rates of target site integration of at least 5%. In some embodiments, constructs comprising homology arms provide rates of target site integration of 1%, 2%, 5%, 10%, 15%, 20%, 25%, 30%, 35%, 40%, 45%, 50%, 55%, 60%, 65%, 70%, 75%, 80%, 85%, 90% or more. In some embodiments, constructs comprising homology arms provide rates of target site integration of 30% or more. In some embodiments, constructs comprising homology arms provide rates of target site integration of 35% or more.

### Targeted Integration

In some embodiments, compositions disclosed herein direct integration of a payload (e.g., a transgene) at a target locus (e.g., an endogenous gene). In some embodiments, compositions and constructs provided herein direct integration of a payload at a target locus in a specific cell type (e.g., naïve B cells, B lineage cell populations, etc.). In some embodiments, compositions and constructs provided herein direct integration of a payload (e.g., expression cassette comprising a transgene encoding FIX) at a target locus in a specific cell type (e.g, naïve B cells, B lineage cell populations, etc.). In some embodiments, a payload is or comprises a transgene or variant thereof encoding FIX, or a variant thereof (e.g., FIX R338L).

In some embodiments, compositions and constructs provided herein direct integration of a payload at a target locus that is considered a safe-harbor site (e.g., CCR5, AAVS1). In some embodiments, a target locus is selected from any genomic site appropriate for use with methods and compositions provided herein. In some embodiments, a target locus encodes a polypeptide. In some embodiments, a target locus encodes a polypeptide that is highly expressed in a subject (e.g., a subject not suffering from a disease, disorder, or condition, or a subject suffering from a disease, disorder, or condition). In some embodiments, a target locus is selected from one or more of CD19, CD20, IGH, B2M, CCR5, JCHAIN, PAX5, IRF4, IRF8, BACH2, EZH2, XBP1, CARD11, PRDM1, and BAFF.

### B Lineage Cell Culture Methods

Various methods for culturing and long-term maintenance of B lineage cells in vitro are described in the art (See, Rawlings et al. 1995, Rawlings et al 1997, and Fluckinger et al. 1998, each of which is incorporated by reference herein in its entirety). B lineage cell culturing conditions can significantly affect normal human B lineage development and for production of mature Ig-secreting B cells. Without wishing to be bound by any theory, it is generally thought that activation of B lineage cells ex vivo may be necessary for later genome editing approaches that make use of homology-directed repair (HDR), as required DNA repair proteins are present during the G2/S phases of the cell cycle (See, Rogers and Cannon 2021, incorporated herein by reference in its entirety).

### Activation of B Lineage Cells

Various technologies for activation of B lineage cells in vitro have been described (also interchangeably referred to as “activated B lineage cells”). Traditionally, B lineage cell activation and proliferation in vitro employed CD40L-expressing feeder cell layer systems. Such feeder cell systems were described as difficult to standardize and often unreliable for providing consistent levels of activation and proliferation of B lineage cells. Recent advances have shifted to protocols for in vitro activation and proliferation of B lineage cells in specific culture systems comprising cytokines or other components in the absence of feeder cells (See Jourdan et al., 2009 and Hartweger et al., 2019, each of which is incorporated herein by reference in its entirety).

In some embodiments, methods for B lineage cell activation comprise contacting cells with media comprising one or more components of the present disclosure. In some embodiments, methods for B lineage cell activation comprise contacting cells with media comprising one or more cytokines and/or oligonucleotides (e.g., multimeric human CD40L, IL-2, IL-10, IL-15, IL-21 and/or CpG). In some embodiments, methods for B cell activation comprise contacting cells with media comprising at least about 5 ng/mL, 10 ng/ml, 15 ng/ml, 20 ng/ml, 25 ng/ml, 30 ng/mL, 35 ng/mL, 40 ng/mL, 45 ng/mL, 50 ng/mL, 55 ng/ml, 60 ng/mL, 65 ng/mL, 70 ng/mL, 75 ng/mL, 80 ng/mL, 85 ng/ml, 90 ng/ml, 95 ng/ml, 100 ng/ml, 150 ng/mL, 200 ng/mL, or 500 ng/ml of one or more cytokines and/or oligonucleotides (e.g., multimeric human CD40L, IL-2, IL-10, IL-15, IL-21 and/or CpG). In some embodiments, methods for B lineage cell activation comprise contacting cells with media comprising at least about 0.1 μg/mL, 0.2 μg/mL, 0.3 μg/mL, 0.4 μg/mL, 0.5 μg/mL, 0.6 μg/mL, 0.7 μg/mL, 0.8 ug/mL, 0.9 μg/mL, 1 μg/mL, 1.5 μg/mL, 2 μg/mL, 2.5 μg/mL, 3 μg/mL, 3.5 μg/mL, 4 μg/mL, 4.5 ug/mL, or 5 μg/mL of one or more cytokines and/or oligonucleotides (e.g., multimeric human CD40L, IL-2, IL-10, IL-15, IL-21 and/or CpG).

In some embodiments, methods for B lineage cell activation comprise a step of contacting cells with media comprising one or more components of the present disclosure for at least about 1, 2, 3, 4, or 5 days. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least about 1, 2, 3, 4, or 5 days. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days, followed by a step of gene editing. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days, followed by a step of B lineage cell expansion. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days, followed by a step of B lineage cell expansion for at least about 1, 2, 3, 4, 5, 6, 7, or 8 days.

In some embodiments, methods described herein result in an activated B lineage cell population.

Differentiation of B Cells into Plasmablasts

Plasmablasts are short-lived, rapidly produced effector cells that are primarily present in an early antibody response and are one potential product of terminal B cell differentiation. Plasmablasts are capable of secreting antibodies, including IgM subtype antibodies, in order to mount an immediate response to certain antigens in the body. Differentiation of B cells into plasmablasts in vitro may be promoted through use of certain signaling molecules, including one or more cytokines (e.g., IL-2, IL-6, IL-10, and/or IL-15). A variety of methods for differentiation of B cells into plasmablasts are known, but not limited to, those outlined in WO/2018/170150, incorporated herein by reference in its entirety. Plasmablasts produced by such methods may be characterized as cells that are CD27+/CD38+/CD1387.

In some embodiments, methods for B cell differentiation into plasmablasts comprise contacting cells with media comprising one or more components. In some embodiments, methods for B cell differentiation into plasmablasts comprise contacting activated B lineage cells with media comprising one or more cytokines (e.g., IL-2, IL-6, IL-10, and/or IL-15). In some embodiments, methods for B cell differentiation into plasmablasts comprise contacting activated B lineage cells with media comprising at least about 0.5 ng/ml, 1 ng/mL, 1.5 ng/mL, 2 ng/mL, 2.5 ng/mL, 3 ng/mL, 3.5 ng/ml, 4 ng/ml, 4.5 ng/ml, 5 ng/mL, 10 ng/ml, 15 ng/mL, 20 ng/mL, 25 ng/ml, 30 ng/mL, 35 ng/ml, 40 ng/mL, 45 ng/mL, 50 ng/ml, 55 ng/mL, 60 ng/mL, 65 ng/mL, 70 ng/mL, 75 ng/mL, 80 ng/ml, 85 ng/ml, 90 ng/ml, 95 ng/ml, or 100 ng/ml of one or more cytokines (e.g., IL-2, IL-6, IL-10, and/or IL-15).

In some embodiments, methods for B cell differentiation into plasmablasts comprise a step of contacting cells with media comprising one or more components of the present disclosure for at least about 1, 2, 3, or 4 days. In some embodiments, methods for B cell differentiation into plasmablasts comprise a step of contacting activated B lineage cells with media comprising one or more cytokines (e.g., IL-2, IL-6, IL-10, and/or IL-15) for at least about 1, 2, 3, or 4 days. In some embodiments, methods for B cell differentiation into plasmablasts comprise a step of contacting activated B lineage cells with media comprising one or more cytokines (e.g., IL-2, IL-6, IL-10, and/or IL-15) for at least 3 days. In some embodiments, methods for B cell differentiation into plasmablasts comprise a step of contacting activated B lineage cells with media comprising one or more cytokines (e.g., IL-2, IL-6, IL-10, and/or IL-15) for at least 3 days, followed by a step of plasmablast expansion.

Differentiation of Plasmablasts into Plasma Cells

Although plasmablasts secrete more antibodies than naïve B cells, they are shorter-lived and secrete fewer antibodies than plasma cells (PCs). Long-lived plasma cells (LLPCs, used interchangeably throughout with plasma cells) localize to bone marrow in the body and are capable of secreting high levels of antibodies and surviving for decades in the absence of proliferation (See, Hammerland et al., 2017 and Khodadadi et al., 2019, both of which incorporated herewith in their entirety). Differentiation of plasmablasts to long-lived plasma cells can be triggered by certain events in the body, including, e.g., activity of transcription factors Blimp-1/PRDM1 and IRF4. Differentiation of plasmablasts to plasma cells in vitro may be promoted through use of certain signaling molecules, including one or more cytokines (e.g., IL-6, IL-15, and/or IFNα-2β). A variety of methods for differentiation of plasmablasts into plasma cells are known, but not limited to, those outlined in Jourdan et al. 2019 and WO/2018/170150 (each of which incorporated herein by reference in its entirety). Plasma cells produced by such methods may be characterized as cells that are CD27⁺/CD38⁺/CD138⁺.

In some embodiments, methods for plasmablast differentiation into plasma cells comprise contacting cells with media comprising one or more cytokines (e.g., IL-6, IL-15, and/or IFNα-2β). In some embodiments, methods for plasmablast differentiation into plasma cells comprise contacting plasmablasts with media comprising one or more cytokines (e.g., IL-6, IL-15, and/or IFNα-2β). In some embodiments, methods for plasmablast differentiation into plasma cells comprise contacting plasmablasts with media comprising at least about 0.5 ng/ml, 1 ng/mL, 1.5 ng/mL, 2 ng/ml, 2.5 ng/ml, 3 ng/ml, 3.5 ng/ml, 4 ng/ml, 4.5 ng/ml, 5 ng/ml, 10 ng/mL, 15 ng/mL, 20 ng/mL, 25 ng/ml, 30 ng/mL, 35 ng/mL, 40 ng/mL, 45 ng/ml, 50 ng/ml, 55 ng/ml, 60 ng/ml, 65 ng/ml, 70 ng/ml, 75 ng/ml, 80 ng/ml, 85 ng/ml, 90 ng/ml, 95 ng/mL, or 100 ng/ml of one or more cytokines (e.g., IL-6, IL-15, and/or IFNα-2β).

In some embodiments, methods for plasmablast differentiation into plasma cells comprise a step of contacting cells with media comprising one or more components of the present disclosure for at least about 1, 2, 3, or 4 days. In some embodiments, methods for plasmablast differentiation into plasma cells comprise a step of contacting plasmablasts with media comprising one or more cytokines (e.g., IL-6, IL-15, and/or IFNα-2β) for at least about 1, 2, 3, or 4 days. In some embodiments, methods for plasmablast differentiation into plasma cells comprise a step of contacting plasmablasts with media comprising one or more cytokines (e.g., IL-6, IL-15, and/or IFNα-2β) for at least 3 days. In some embodiments, methods for plasmablast differentiation into plasma cells comprise a step of contacting plasmablasts with media comprising one or more cytokines (e.g., IL-6, IL-15, and/or IFNα-2β) for at least 3 days, followed by a step of cell isolation. In some embodiments, methods for plasmablast differentiation into plasma cells comprise a step of contacting plasmablasts with media comprising one or more cytokines (e.g., IL-6, IL-15, and/or IFNα-2β) for at least 3 days, followed by a step of administration to a subject.

### Engineered Cell Preparations

The present disclosure describes engineered cell preparations comprising populations of cells modified to perform one or more desired functions. In some embodiments, engineered cell preparations are compositions comprising genetically modified immune cell populations (e.g., B cell, T cell). In some embodiments, engineered cell preparations are compositions comprising genetically modified B lineage cell populations (e.g., B cell, plasmablast, plasma cell). In some embodiments, engineered cell preparations are compositions comprising genetically modified plasmablast cell populations. In some embodiments, engineered cell preparations are compositions comprising genetically modified plasma cell populations.

In some embodiments, engineered cell preparations are genetically modified to express a payload (e.g., transgene) of interest. In some embodiments, engineered cell preparations are genetically modified to express a transgene from an expression cassette (e.g., comprising additional polynucleotide sequence elements).

In some embodiments, engineered cell preparations comprise genetically modified cells that express a transgene of interest (e.g., therapeutic protein, antibody, etc.). In some embodiments, engineered cell preparations comprise genetically modified cells that express a transgene of interest (e.g., therapeutic protein, antibody, etc.) from an endogenous gene locus. In some embodiments, engineered cell preparations comprise genetically modified cells that express a transgene of interest (e.g., therapeutic protein, antibody, etc.) from an endogenous gene locus under control of an endogenous promoter. In some embodiments, engineered cell preparations comprise genetically modified cells that express a transgene of interest (e.g., therapeutic protein, antibody, etc.) from an endogenous gene locus under control of an exogenous promoter. In some embodiments, engineered cell preparations comprise genetically modified cells that express a transgene of interest (e.g., therapeutic protein, antibody, etc.) from an endogenous gene locus without disrupting endogenous gene expression and/or function. In some embodiments, engineered cell preparations comprise genetically modified cells that express a transgene of interest (e.g., therapeutic protein, antibody, etc.) from an endogenous gene locus and partially or fully disrupt endogenous gene expression and/or function.

In some embodiments, engineered cell preparations are genetically modified to express a transgene from an expression cassette, wherein the expression cassette further comprises one or more polynucleotide sequences encoding one or more promoters. In some embodiments, engineered cell preparations are genetically modified to express a transgene from an expression cassette, wherein the expression cassette further comprises one or more polynucleotide sequences encoding one or more enhancers. In some embodiments, engineered cell preparations are genetically modified to express a transgene from an expression cassette, wherein the expression cassette further comprises one or more polynucleotide sequences encoding one or more terminators. In some embodiments, engineered cell preparations are genetically modified to express a transgene from an expression cassette, wherein the expression cassette further comprises one or more polynucleotide sequences encoding one or more homology arms. In some embodiments, engineered cell preparations are genetically modified to express a transgene from an expression cassette, wherein the expression cassette further comprises one or more polynucleotide sequences encoding one or more promoters, one or more enhancers, one or more terminators, and/or one or more homology arms. In some embodiments, engineered cell preparations are genetically modified to express an FIX transgene, or variant thereof, from an expression cassette, wherein the expression cassette further comprises one or more polynucleotide sequences encoding a MND promoter, a WPRE enhancer, a BGH polyA, a 5′ homology arm, and a 3′ homology arm. In some embodiments, engineered cell preparations are genetically modified to express an FIX transgene, or variant thereof, from an expression cassette, wherein the expression cassette further comprises one or more polynucleotide sequences encoding a MND promoter, a BGH polyA, a 5′ homology arm, and a 3′ homology arm. In some embodiments, engineered cell preparations are genetically modified to express an FIX transgene, or variant thereof, from an expression cassette, wherein the expression cassette further comprises one or more polynucleotide sequences encoding a MND promoter, a WPRE enhancer, a SV40 polyA, a 5′ homology arm, and a 3′ homology arm. In some embodiments, engineered cell preparations are genetically modified to express an FIX transgene, or variant thereof, from an expression cassette, wherein the expression cassette further comprises one or more polynucleotide sequences encoding a MND promoter, a SV40 polyA, a 5′ homology arm, and a 3′ homology arm. In some embodiments, engineered cell preparations are genetically modified to express an FIX transgene, or variant thereof, from an expression cassette, wherein the expression cassette further comprises one or more polynucleotide sequences encoding a EF-1a promoter, a WPRE enhancer, a BGH polyA, a 5′ homology arm, and a 3′ homology arm. In some embodiments, engineered cell preparations are genetically modified to express an FIX transgene, or variant thereof, from an expression cassette, wherein the expression cassette further comprises one or more polynucleotide sequences encoding a EF-1a promoter, a BGH polyA, a 5′ homology arm, and a 3′ homology arm. In some embodiments, engineered cell preparations are genetically modified to express an FIX transgene, or variant thereof, from an expression cassette, wherein the expression cassette further comprises one or more polynucleotide sequences encoding an EF-1a promoter, a SV40 polyA, a 5′ homology arm, and a 3′ homology arm.

### Production

The present disclosure describes production of certain engineered B lineage cell preparations. As disclosed herein, in some embodiments, a B lineage cell preparation comprises both engineered and non-engineered cells. In some embodiments, an engineered B lineage cell population comprises plasmablasts. In some embodiments, an engineered B lineage cell population comprises plasma cells. In some embodiments, an engineered B lineage cell population comprises long-lived plasma cells. In some embodiments, an engineered B lineage cell population comprises plasmablasts, plasma cells, and/or long-lived plasma cells and/or any mixtures or combinations thereof disclosed herein.

### Plasmablast Preparation

In some embodiments, an engineered B lineage cell preparation comprises plasmablasts. As understood in the art, plasmablasts are rapidly produced and short-lived effector cells of the early antibody response. Plasmablasts can be generated from activated B lineage cells using methods described herein. In some embodiments, engineered plasmablasts may be generated from engineered activated B lineage cells.

In some embodiments, plasmablast cell populations may be contacted with media comprising one or more components of the present disclosure (e.g., IL-6, IL-15, and/or IFNα-2β) in order to initiate differentiation into plasma cell populations. In some embodiments, methods for plasmablast cell population differentiation into a plasma cell population comprise contacting plasmablast cell population with media comprising one or more cytokines (e.g., IL-6, IL-15, and/or IFNα-2β). In some embodiments, methods for a plasmablast cell population differentiation into a plasma cell population comprise contacting plasmablast cell population with media comprising at least about 0.5 ng/mL, 1 ng/mL, 1.5 ng/ml, 2 ng/mL, 2.5 ng/ml, 3 ng/ml, 3.5 ng/ml, 4 ng/mL, 4.5 ng/mL, 5 ng/mL, 10 ng/mL, 15 ng/ml, 20 ng/ml, 25 ng/ml, 30 ng/mL, 35 ng/mL, 40 ng/mL, 45 ng/mL, 50 ng/ml, 55 ng/ml, 60 ng/ml, 65 ng/ml, 70 ng/mL, 75 ng/ml, 80 ng/mL, 85 ng/ml, 90 ng/mL, 95 ng/mL, or 100 ng/ml of one or more cytokines (e.g., IL-6, IL-15, and/or IFNα-2β).

In some embodiments, methods for plasmablast cell population differentiation into a plasma cell population comprise a step of contacting cells with media comprising one or more components of the present disclosure for at least about 1, 2, 3, or 4 days. In some embodiments, methods for plasmablast cell population differentiation into a plasma cell population comprise a step of contacting plasmablast cell population with media comprising one or more cytokines (e.g., IL-6, IL-15, and/or IFNα-2β) for at least about 1, 2, 3, or 4 days. In some embodiments, methods for plasmablast cell population differentiation into a plasma cell population comprise a step of contacting plasmablast cell population with media comprising one or more cytokines (e.g., IL-6, IL-15, and/or IFNα-2β) for at least 3 days. In some embodiments, methods for plasmablast cell population differentiation into a plasma cell population comprise a step of contacting plasmablast cell population with media comprising one or more cytokines (e.g., IL-6, IL-15, and/or IFNα-2β) for at least 3 days, followed by a step of cell isolation. In some embodiments, methods for plasmablast cell population differentiation into a plasma cell population comprise a step of contacting plasmablasts with media comprising one or more cytokines (e.g., IL-6, IL-15, and/or IFNα-2β) for at least 3 days, followed by a step of administration to a subject.

### Plasma Cell Preparation

In some embodiments, an engineered B lineage cell preparation comprises a plasma cell population. In some embodiments, a plasma cell population comprises predetermined engineered plasma cell precursors (e.g., plasma blasts) that, upon administration to a subject, further differentiates into a mature plasma cell population. As understood in the art, a mature plasma cell population comprises quiescent, non-dividing cells of the humoral immune response that are capable of secreting large amounts of antibodies. In some embodiments, a mature plasma cell population can comprise short-lived plasma cells and/or long-lived plasma cells (LLPCs) and/or any combination thereof.

### Characterization

The present disclosure provides various methods for characterization of engineered cell populations (e.g., B lineage cell populations). In some embodiments, methods disclosed herein are used to segregate and/or characterize naïve B cell subpopulations within a B lineage cell population. In some embodiments, naïve B cell subpopulations are characterized through one or more of flow cytometry, fluorescence-activated cell sorting (FACS), magnetic-activated cell sorting (MACS), and/or affinity chromatography. In some embodiments, one or more methods of characterizing naïve B cell subpopulations are employed during each step of a controlled cooling method. In some embodiments, methods of characterization are employed to segregate and/or characterize engineered naïve B cell subpopulations. In some embodiments, engineered naïve B cell subpopulations are characterized through one or more of flow cytometry, fluorescence-activated cell sorting (FACS), magnetic-activated cell sorting (MACS), and/or affinity chromatography. In some embodiments, one or more methods of characterizing engineered naïve B cell subpopulations are employed during each step of a controlled cooling method.

In some embodiments, methods disclosed herein are used to segregate and/or characterize activated B lineage cell subpopulations within a B lineage cell population. In some embodiments, activated B lineage cell subpopulations are characterized through one or more of flow cytometry, fluorescence-activated cell sorting (FACS), magnetic-activated cell sorting (MACS), and/or affinity chromatography. In some embodiments, one or more methods of characterizing activated B lineage cell subpopulations are employed during each step of a controlled cooling method. In some embodiments, methods disclosed herein are used to segregate and/or characterize engineered activated B lineage cell subpopulations. In some embodiments, engineered activated B lineage cell subpopulations are characterized through one or more of flow cytometry, fluorescence-activated cell sorting (FACS), magnetic-activated cell sorting (MACS), and/or affinity chromatography. In some embodiments, one or more methods of characterizing engineered activated B lineage cell subpopulations are employed during each step of a controlled cooling method.

In some embodiments, methods disclosed herein are used to segregate and/or characterize plasmablast cell subpopulations within a B lineage cell population. In some embodiments, plasmablast cell subpopulations are characterized through one or more of flow cytometry, fluorescence-activated cell sorting (FACS), magnetic-activated cell sorting (MACS), and/or affinity chromatography. In some embodiments, one or more methods of characterizing plasmablast cell subpopulations are employed during each step of a controlled cooling method. In some embodiments, methods disclosed herein are used to segregate and/or characterize engineered plasmablast cell subpopulations. In some embodiments, engineered plasmablast cell subpopulations are characterized through one or more of flow cytometry, fluorescence-activated cell sorting (FACS), magnetic-activated cell sorting (MACS), and/or affinity chromatography. In some embodiments, one or more methods of characterizing engineered plasmablast cell subpopulations are employed during each step of a controlled cooling method.

In some embodiments, methods disclosed herein are used to segregate and/or characterize plasma cell precursor subpopulations within a B lineage cell population. In some embodiments, plasma cell precursor subpopulations are characterized through one or more of flow cytometry, fluorescence-activated cell sorting (FACS), magnetic-activated cell sorting (MACS), and/or affinity chromatography. In some embodiments, one or more methods of characterizing plasma cell precursor subpopulations are employed during each step of a controlled cooling method. In some embodiments, methods disclosed herein are used to segregate and/or characterize engineered plasma cell precursor subpopulations. In some embodiments, engineered plasma cell precursor subpopulations are characterized through one or more of flow cytometry, fluorescence-activated cell sorting (FACs), magnetic-activated cell sorting (MACs), and/or affinity chromatography. In some embodiments, one or more methods of characterizing engineered plasma cell precursor subpopulations are employed during each step of a controlled cooling method.

In some embodiments, methods disclosed herein are used to segregate and/or characterize plasma cell subpopulations within a B lineage cell population. In some embodiments, plasma cell subpopulations may be characterized through one or more of flow cytometry, fluorescence-activated cell sorting (FACS), magnetic-activated cell sorting (MACS), and/or affinity chromatography. In some embodiments, one or more methods of characterizing plasma cell subpopulations are employed during each step of a controlled cooling method. In some embodiments, methods disclosed herein are used to segregate and/or characterize engineered plasma cell subpopulations. In some embodiments, engineered plasma cell subpopulations are characterized through one or more of flow cytometry, fluorescence-activated cell sorting (FACS), magnetic-activated cell sorting (MACS), and/or affinity chromatography. In some embodiments, one or more methods of characterizing engineered plasma cell subpopulations are employed during each step of a controlled cooling method.

In some embodiments, methods disclosed herein are used to segregate and/or characterize short-lived plasma cell populations within a B lineage cell population. In some embodiments, short-lived plasma cell subpopulations are characterized through one or more of flow cytometry, fluorescence-activated cell sorting (FACS), magnetic-activated cell sorting (MACS), and/or affinity chromatography. In some embodiments, one or more methods of characterizing short-lived plasma cell subpopulations are employed during each step of a controlled cooling method. In some embodiments, methods disclosed herein are used to segregate and/or characterize engineered short-lived plasma cell subpopulations. In some embodiments, engineered short-lived plasma cell subpopulations are characterized through one or more of flow cytometry, fluorescence-activated cell sorting (FACS), magnetic-activated cell sorting (MACS), and/or affinity chromatography. In some embodiments, one or more methods of characterizing engineered short-lived plasma cell subpopulations are employed during each step of a controlled cooling method.

In some embodiments, methods disclosed herein may be used to segregate and/or characterize long-lived plasma cell subpopulations with a B lineage cell population. In some embodiments, long-lived plasma cell subpopulations are characterized through one or more of flow cytometry, fluorescence-activated cell sorting (FACS), magnetic-activated cell sorting (MACS), and/or affinity chromatography. In some embodiments, one or more methods of characterizing long-lived plasma cell subpopulations are employed during each step of a controlled cooling method. In some embodiments, methods disclosed herein are used to segregate and/or characterize engineered long-lived plasma cell subpopulations. In some embodiments, engineered long-lived plasma cell subpopulations are characterized through one or more of flow cytometry, fluorescence-activated cell sorting (FACS), magnetic-activated cell sorting (MACS), and/or affinity chromatography. In some embodiments, one or more methods of characterizing engineered long-lived plasma cell subpopulations are employed during each step of a controlled cooling method.

### Methods of Treatment

The present disclosure, among other things, provides methods of treating a disease, disorder, or condition (e.g., a disease, disorder, or condition described herein) in a subject comprising administering a pharmaceutical composition described herein. In some embodiments, a therapeutically effective amount of a pharmaceutical composition described herein is administered to a subject having a disease or disorder. Pharmaceutical compositions described herein can be for use in the manufacture of a medicament for treating a disease, disorder, or condition (e.g., a disease, disorder, or condition described herein) in a subject.

Pharmaceutical compositions described herein can comprise one or more B lineage cells selected from a population of genetically modified B lineage cells described herein. In some embodiments, B lineage cell populations are engineered to express or comprise a payload. In some embodiments, a payload is or comprises an expression cassette. In some embodiments, an expression cassette comprises a transgene encoding FIX.

A subject to be treated with methods described herein can be a mammal, e.g., a primate, e.g., a human (e.g., a patient having, or at risk of having, a disease, disorder, or condition described herein). In some embodiments, a subject has hemophilia (e.g., hemophilia B). In some embodiments, a subject can be an adult subject. In some embodiments, engineered B lineage cells are administered to a pediatric subject.

Administration of pharmaceutical compositions described herein may be carried out in any convenient manner (e.g., injection, ingestion, transfusion, inhalation, implantation, or transplantation). In some embodiments, a pharmaceutical composition described herein is administered by injection or infusion. Pharmaceutical compositions described herein may be administered to a subject intravenously transarterially, subcutaneously, intradermally, intratumorally, intranodally, intramedullary, intramuscularly, or intraperitoneally. In some embodiments, a pharmaceutical composition described herein is administered parenterally (e.g., intravenously, subcutaneously, intraperitoneally, or intramuscularly). In some embodiments, a pharmaceutical composition described herein is administered by intravenous infusion or injection. In some embodiments, a pharmaceutical composition described herein is administered by intramuscular or subcutaneous injection.

In some embodiments, a pharmaceutical composition described herein is administered at a pharmaceutically suitable dosage to a subject. In some embodiments, a pharmaceutical composition described herein is administered monthly. In some embodiments, a pharmaceutical composition described herein is administered once every other month. In some embodiments, a pharmaceutical composition described herein is administered once every three months. In some embodiments, a pharmaceutical composition described herein is administered once every six months. In some embodiments, a pharmaceutical composition described herein is administered once a year. In some embodiments, a pharmaceutical composition described herein is administered to a subject having hemophilia B. In some embodiments, a pharmaceutical composition described herein is administered to a subject with mild-to-severe hemophilia B.

In some embodiments, the methods disclosed herein comprise measuring and/or monitoring treatment of hemophilia B. In some embodiments, a sample is collected from a subject treated with a pharmaceutical composition described herein. In some embodiments, sample collection is or comprises venipuncture. In some embodiments, sample collection is or comprises tissue collection. In some embodiments, levels of FIX are measured from sample collection. In some embodiments, lipids are assessed from a collected sample.

In some embodiments, engraftment of one or more B lineage cells of a population of genetically modified B lineage cells described herein is measured in a non-clinical species (e.g., hIL6-/NOG mice). In some embodiments, engraftment of one or more B lineage cells of a population of genetically modified B lineage cells described herein is measured in a clinical species (e.g., a human subject). In some embodiments, engraftment is measured by use of bioluminescence. In some embodiments, engraftment is measured by use of ELISpot. In some embodiments, levels of plasma IgG are determined to measure engraftment. In some embodiments, levels of plasma IgM are determined to measure engraftment. In some embodiments, levels of a transgene (e.g., FIX) are determined to measure engraftment. In some embodiments, engraftment of one or more B lineage cells of an engineered B lineage cell population occurs in a non-clinical species (e.g., hIL6-/NOG mice) with or without preconditioning of a non-clinical subject (e.g., chemotherapy, immunosuppressive treatment, etc. of a subject prior to administering B lineage cells as described herein). In some embodiments, engraftment of one or more B lineage cells of an engineered B lineage cell population described herein occurs in a clinical species (e.g., human subject) with or without preconditioning of a clinical subject (e.g., chemotherapy, immunosuppressive treatment, etc. of a subject prior to administering B lineage cells as described herein).

### EXAMPLES

The following examples are provided so as to describe to the skilled artisan how to make and use methods and compositions described herein and are not intended to limit the scope of the present disclosure.

### Example 1: Materials and Methods

The present Example demonstrates in vitro assay methods that may be implemented to certain cell populations, including, but not limited to, engineered B lineage cell populations. Cell populations can comprise or be engineered B lineage cell populations that express FIX. Engineered B lineage cell populations comprise or be naïve B cells. Engineered B lineage cell populations comprise or be engineered activated B lineage cells. Engineered B lineage cell populations may comprise or be engineered plasmablast. Engineered B lineage cell populations may comprise or be engineered plasma cells.

### Culturing Methods (Serum Variation)

The present application provides methods that result in engineered B lineage cell populations. These methods include, but are not limited to, serum variation methods described herein.

In some embodiments, B lineage cells are isolated and cultured in media comprising one or more cytokines and/or oligonucleotides (e.g., CD40L, IL-2, IL-10, IL-15, IL-21 and/or CpG) for at least about 1, 2, 3, 4, or 5 days. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days, followed by a step of gene editing.

In some embodiments, B lineage cells are contacted with media lacking serum (e.g., human serum, bovine serum, horse serum, newborn calf serum, goat serum, rabbit serum, porcine serum, chicken serum, or a combination thereof) during gene editing. In some embodiments, B lineage cells subsequently undergo a wash step with base media (e.g., Excellerate) with or without cytokines. In some embodiments, after gene editing, B lineage cells are contacted with media lacking serum (e.g., human serum, bovine serum, horse serum, newborn calf serum, goat serum, rabbit serum, porcine serum, chicken serum, or a combination thereof) and then incubated at 37° C. for up to 24 hours. In some embodiments, after gene editing, B lineage cells are contacted with media substantially free of serum (e.g., human serum, bovine serum, horse serum, newborn calf serum, goat serum, rabbit serum, porcine serum, chicken serum, or a combination thereof) and then incubated at 37° C. for up to 24 hours. In some embodiments, B lineage cells after incubation are spun down, supernatant removed, and replated in media comprising serum (e.g., human serum, bovine serum, horse serum, newborn calf serum, goat serum, rabbit serum, porcine serum, chicken serum, or a combination thereof). In some embodiments, B lineage cells are replated in media comprising one or more cytokines and/or oligonucleotides (e.g., CD40L, IL-2, IL-10, IL-15, IL-21 and/or CpG) and expanded for at least 5 days.

ELISA to Detect Human FIX from Engineered B Lineage Cell Population

Engineered B lineage cell population supernatant was harvested from relevant experiments and frozen at −80° C. prior to testing. Samples were then tested for concentrations of human Factor IX (hFIX) in the 96 well FIX ELISA (Abcam-ab108831). Briefly, all kit components, samples, and standards were thawed and allowed to equilibrate to room temperature. 50 μL of sample or standard were added per well of pre-coated plates and then incubated at room temperature for 2 hours. After incubation, plates were washed 5 times with 200 μL of 1×Wash Buffer. Next, 50 μL of 1×biotinylated FIX detector antibody was added into each well and incubated for 1 hour at room temperature. Plates were then washed and 50 μL of 1×SP conjugate was added to each well. After addition of 1×SP conjugate, plates were then incubated for 30 minutes at room temperature. After another round of plate washing, 50 μL of Chromogen Substrate was added per well and incubated for 10 minutes at room temperature. After color develops in the wells, 50 μL of Stop Solution was added to quench. The plate absorbance was read at 450 nm and 570 nm by a Cytation5 Plate Reader. Sample values were interpolated using the standards at known hFIX concentrations.

### Immunoglobulin G (IgG) and Immunoglobulin M (IgM) ELISA of Engineered B Lineage Cell Population Supernatant and Mouse Plasma

Briefly, 384 well maxisorp plates were coated with capture antibody (anti-human IgG at 3 μg/mL or anti-human IgM at 1 μg/mL in carb-bicarb buffer) overnight at 4° C. The plates were then washed 3 times with an automated plate washer. The antibody-coated plates were blocked against non-specific binding with 1% BSA for 1 hour at room temperature. Plasma samples or standards were added to the wells of the plate and incubated for 1.5 hours at room temperature. Plates were washed as previously described. Plate-bound hIgG or hIgM was then captured by secondary antibody, goat anti-IgG polyclonal antibody HPR or goat anti-IgM polyclonal antibody HPR respectively, at 1:5000 dilution for 1 hour at room temperature. Next, TMB two-part substrate was added for 1 min (development time to be determined empirically) and then the reaction was stopped. The plate absorbance was read at 450 nm and 570 nm by a Cytation5 Plate Reader. Sample values were interpolated using the standards at known human IgG or human IgM concentrations.

### Factor IX ELISA In Mouse Plasma

384 well maxisorp plates were coated with capture antibody (GMA102 antibody at 3 μg/mL in carb-bicarb buffer) overnight at 4° C., and then plates were washed 3 times with an automated plate washer. The antibody-coated plates were blocked against non-specific binding with 1% BSA for 1 hour at room temperature. Plasma samples or standards, BeneFIX® (Pfizer), were added to the wells of the plate and incubated for 1 hour at room temperature. Plates were washed as previously described. Plate-bound FIX was then captured by secondary antibody, 1:500 dilution of biotinylated (mouse-adsorbed) FIX polyclonal antibody for 1 hour at room temperature. This was then followed by incubation of poly-HRP Streptavidin at 1:5,000 dilution for 1 hour at room temperature. TMB two-part substrate was added for 1 to 2 min and then the reaction was stopped. The plate absorbance was read at 450 nm and 570 nm by a Cytation5 Plate Reader. Sample values were interpolated using the standards at known human FIX concentrations.

### Immunoglobulin G (IgG) and Immunoglobulin M (IgM) ELISpot of Engineered B Lineage Cell Population Supernatant

96 well multiscreen filter plates were briefly washed with 35% ethanol for 40-60 seconds until the membrane turned gray from white. Plates were then washed 3 times with Dulbecco's PBS (dPBS), and subsequently coated with the capture antibody (anti-human IgG or anti-human IgM) at 10 μg/mL in PBS overnight at 4° C. Next day, the plates were washed 3 times with an automated plate washer. The antibody coated plates were then blocked against non-specific binding with Iscove's Modified Dulbecco's Medium (IMDM) with 1% FBS for 2 hours at 37° C. Next, cells were counted and added to the wells of the plate. The plates were incubated in an incubator at 37° C. for 16-24 hours. Next day, the plates were washed for an additional 3 times. Plates were then blocked with 1% BSA at room temperature for 1 hour and washed 3 more times. Next, plate-bound hIgG or hIgM were detected by a secondary biotinylated antibody (anti-human IgG or anti-human IgM) at 1:1000 dilution in 1% BSA and were incubated for 2 hours at room temperature. At the end of incubation, plates were washed as previously described and then with exposed to streptavidin alkaline phosphatase at 1:5000 dilution in 1% BSA. Plates were subsequently incubated at room temperature for 1 hour before undergoing an additional 3 washes. 5-Bromo-4-Chloro-Indolyl-Phosphatase (BCIP) and Nitroblue Tetrazolium (NBT) was then added to the plates for 5-15 minutes, before being washed with tap water. Plates were then tapped onto a blotting paper and dried in the dark overnight. Once the plates are dried, they were read on the AID plate reader and analyzed for the number of either human IgG or IgM producing spots.

### Factor XI (FIX) ELISpot Of Engineered B Lineage Cell Population Supernatant

96 well multiscreen filter plates were briefly washed with 35% ethanol for 40-60 seconds until the membrane turned gray from white. Plates were then washed 3 times with Dulbecco's PBS (dPBS), and subsequently coated with the capture antibody (GMA102) at 30 μg/mL in PBS overnight at 4° C. The next day, plates were washed 3 times with an automated plate washer. The antibody coated plates were then blocked against non-specific binding with Iscove's Modified Dulbecco's Medium (IMDM) with 1% FBS for 2 hours at 37° C. Next, cells were counted and added to the wells of the plate. The plates were incubated in an incubator at 37° C. for 16-24 hours. Next day, the plates were washed for an additional 3 times. Plates were then blocked with 1% BSA at room temperature for 1 hour and washed 3 more times. Next, plate-bound FIX was then detected by a secondary antibody (rabbit biotinylated polyclonal anti-FIX antibody) at 1:1000 dilution in 1% BSA and were incubated for 2 hours at room temperature. At the end of incubation, plates were washed as previously described. Then in order to amplify the signal, a biotinylated sheep anti-rabbit tertiary antibody was added at 1:1000 dilution in 1% BSA and plates were incubated for 1 hour at room temperature. Plates were then washed as previously described and streptavidin alkaline phosphatase was added at 1:5000 dilution in 1% BSA. Plates were subsequently incubated at room temperature for 1 hour before undergoing an additional 3 washes. 5-Bromo-4-Chloro-Indolyl-Phosphatase (BCIP) and Nitroblue Tetrazolium (NBT) was then added to the plates for 5-15 minutes, before being washed with tap water. Plates were then tapped onto a blotting paper and dried in the dark overnight. Once the plates are dried, they were read on the AID plate reader and analyzed for the number of FIX producing spots.

### FIX Immunocapture Activity Assay for Engineered B Lineage Cell Population Supernatant

In order to evaluate the FIX activity of engineered B lineage cell populations as disclosed herein, a combination of a Factor IX immunocapture chromogenic activity assay with a capture antibody (mouse monoclonal anti-human FIX light chain: Green Mountain Antibodies, cat #GMA184) with the Factor IX chromogenic activity assay kit, (Rox Factor IX, Diapharma cat #900020) was developed. These two methods were used in conjunction to determine the activity of FIX produced from engineered B lineage cell population (BeCM) supernatants. The Rox FIX activity kit is a chromogenic assay based upon FXa generation for the determination of FIX activity in preparations containing Factor IX.

Briefly, 384 well maxisorp plates were coated with 10 μg/mL GMA-184 capture anitbody (anti-human FIX light chain antibody) overnight at 4° C. then plates were washed 3 times with an automated plate washer. The antibody-coated plates were blocked against non-specific binding with 1% BSA. Samples, supernatants from engineered B lineage cell populations or standards, BeneFIX® (Pfizer) were added to the wells of the plate and incubated for 2-4 hours at room temperature. Plates were washed as previously described. Plate-bound FIX was then evaluated for activity by the Rox Factor IX chromogenic activity kit (Rox 900020) using a modification of reagent volumes to match the size of the plate. After quenching the enzymatic reaction, the plate absorbance was read at 405 nm by a Cytation5 Plate Reader. Sample activity values were interpolated using the standards at known activity concentrations. Results reported as hFIX-specific activity in International Units (IU) normalized to total FIX, as determined by ELISA of supernatant.

### Inference of CRISPR Editing (ICE)

Cells were collected 72 hours after electroporation from non-engineered control (reference template) and Ribonucleoprotein (RNP)-only engineered conditions. The Maxwell DNA extraction kit was used to extract genomic DNA and normalized to 16.5 ng/ml after quantitation on the NanoDrop Spectrometer. Primers specific to the CRISPR sgRNA site were designed such that the target cut site was ~200 bp downstream the sequencing primer and ~600 bp upstream of the amplicon terminus. PCR amplification was performed using Q5 HiFi Hot start 2× MasterMix and the recommended thermocycler program. A no-template control was included. Amplicon sizes were checked using the Lonza FlashGel system and shipped for Sanger Sequencing. Analysis of the Sanger sequences traces was performed using the Synthego ICE analytical method run off an internal server.

ICE Forward primer:
(SEQ ID NO: 1)
GCAGCAAACCTTCCCTTCACTAC
ICE Reverse primer:
(SEQ ID NO: 2)
AGGATTCCCGAGTAGCAGATGAC
ICE Sequencing primer:
(SEQ ID NO: 3)
GGGTGGAACAAGATGGATTATC

Droplet Digital Polymerase Chain Reaction (ddPCR)

ddPCR enables quantification of targeted integration efficiency as follows: copies/μL NHEJ-sensitive probe (FAM)+droplets (integration site)/copies/μL NHEJ-insensitive probe (HEX)+droplets (reference amplicon)*100=% Targeted integration.

For the integration site amplicon, one primer lies outside of a homology arm and the other lies inside the transgene. Thus, an amplicon is only generated when a genomic integration event has occurred. The reference amplicon is ideally a similar size and generated from either a distal region on the same locus or a reference gene. Amplicon primers were designed using the IDT PrimerQuest tool, and ddPCR probes were designed according to the following rules:
1) Designed the FAM with its its 5′ end directly in the hotspot of the mutations, melting temperature (Tm)=56-57° C.
2) Designed the NHEJ-insensitive probe (HEX) within the amplicon where it is not likely to be mutated, Tm=60° C.
3) Designed primers to produce a 60-150 nt amplicon with 40-60% GC content.
4) Primer length should be 18-24 nt with a GC content of 50-60%, Tm=55 C (50-65° C. acceptable).
5) Included three controls: no-template (excludes contamination), untreated (aids gating), transfection without gRNA (assess impact of endonuclease expression).

Reaction mix (including replicates) and ddPCR Supermix for Probes (no dUTP, BioRad) were allowed to equilibrate to room temperature. Droplets were generated using the Automated Droplet Generator following the manufacturer's instructions (BioRad). Reactions were then run in the C1000 Touch Thermal Cycler program recommended for the amplicon length. Samples were then read on a QX200 Droplet Reader (BioRad) and analyzed on 2D Amplitude (dot plot) for discrimination between negative, single, and double-positive droplets. Thresholding was done by calculating: Average of negative droplet intensity+0.1×(Average of positive droplet intensity-average of negative droplet intensity)=Threshold.

% Targeted integration was then calculated using the above equation based on the adjusted thresholding and the copies/uL of FAM and HEX.

The following CCR5 target gene primers and probes were used:

CCR5 ddPCR Forward primer:
(SEQ ID NO: 4)
catcgcattgtctgagtagg.
CCR5 ddPCR Reverse primer:
(SEQ ID NO: 5)
CAGTGGATCGGGTGTAAAC.
CCR5_ddPCR Probe (FAM):
(SEQ ID NO: 6)
TCGGGAGCCTCTTGCTGGAAAATAGAA.

The following CCRL2 reference gene primers and probes were used:

CCRL2_ddPCR Forward primer:
(SEQ ID NO: 7)
CCACATCAGAAGGAAGACTAC.
CCRL2_ddPCR Reverse primer:
(SEQ ID NO: 8)
GCTGTATGAATCCAGGTCC.
CCRL2_ddPCR Probe (HEX):
(SEQ ID NO: 9)
TGTTTCCTCCAGGATAAGGCAGCTGT.

### Cell Count and Viability

Cells were sampled for the CellacaMx counter after thorough resuspension by gentle pipetting. Cells were gently mixed with the appropriate diluent of AOPI stain, loaded into a Nexcelom count plate, and analyzed for count and viability based on the acquired image and dilution factor.

### Flow Cytometry Analysis

Following resuspension of the cell samples, 2×10⁵ cells were set-aside for flow cytometric analysis. Samples were washed and stained with antibody cocktails focused on immune cell purity (markers: CD19, CD20, CD16, CD56, CD3, CD14, CD45), on B lineage cell phenotyping (markers: CD19, CD20, CXCR4, CD27, CD38, CD138), or on Ig profiling (markers: CD38, CD138, IgM, IgG, IgE, IgA). Cells were washed once in PBS, then stained with LIVE/DEAD Fixable Near-IR Dead Cell Stain (Life Technologies) for 10 minutes at room temperature. Cells were washed with Flow Cytometry Staining Buffer (eBioscience) and resuspended in Human TruStain FcX (BioLegend). After 5 minutes of Fc blocking at 4° C., samples were then resuspended in 100 μL of antibody cocktail master mix. Samples were incubated for 20 minutes at 4° C. in the dark, then washed two times before being resuspended in 400 μL staining buffer for acquisition on the flow cytometer. Single color controls for voltage gating and compensation were also prepared. Following compensation as guided by the NovoCyte software prompt, >10,000 cellular events were acquired per sample. Files were saved and imported into FlowJo. Live cell singlets were further gated in this software, then population percentages, total events, and geometric mean fluorescence intensity values were exported, formatted, and statistically analyzed.

For IgM/IgG profiling, intracellular staining was performed using cell fixation and permeabilization. Following the last wash from viability staining, cells were suspended in fixation buffer (4% paraformaldehyde) and incubated at room temperature for 20 minutes. Cells were then washed in Permeabilization Buffer (eBioscience) three times, then resuspended in Ig profiling antibody master mix (dilutions used were manufacturer's recommendation). Samples were incubated 20 minutes at room temperature protected from light, then washed 2 times with permeabilization buffer. Pellets were suspended in staining buffer as for fresh cell staining and run with forward/side-scatter gating adjusted for cell shrinkage after fixation.

Activated Partial Thromboplastin Clotting Time (aPPT) Assay

B lineage cell populations were cultured and engineered as described above. On Day 13 of culture, 100 ng/ml of menadione sodium bisulfite was added to B lineage cells over a time course of about 24 hours. Harvested supernatant was purified using an FIX affinity resin. Purified FIX was then spiked into FIX-deficient plasma. Clotting times were generated on the Stago Satellite, recorded, and interpolated against an activity curve generated with normal pooled plasma and the same FIX-deficient plasma. Corresponding clotting times were converted to percent activity using a log-log regression. This results in % activity and IU/mL and IU/mg which was then recorded accordingly.

### Example 2: Engineered B Lineage Cell Populations can Integrate, Express and/or Secrete Proteins of Interest

The present Example demonstrates that B lineage cell populations engineered with one or more expression cassettes (e.g., FIX containing expression cassette) may integrate a polynucleotide sequence encoding for a transgene and express said transgene (e.g., FIX). Subjects treated with engineered cell populations described herein may demonstrate persistent levels of transgene expression (e.g., Factor IX) over an extended period of time (e.g., one or more weeks, one or more months, etc.).

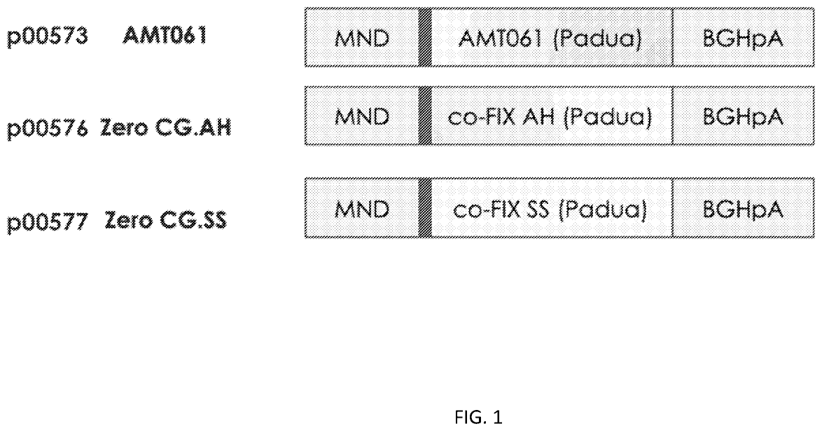

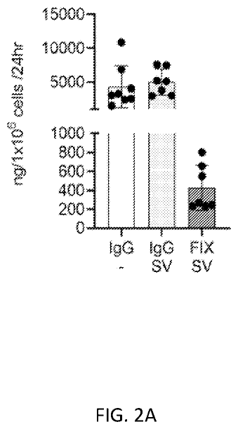

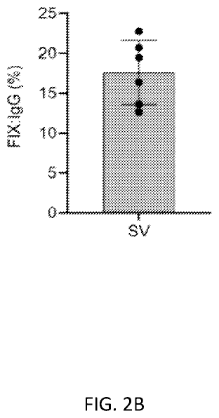

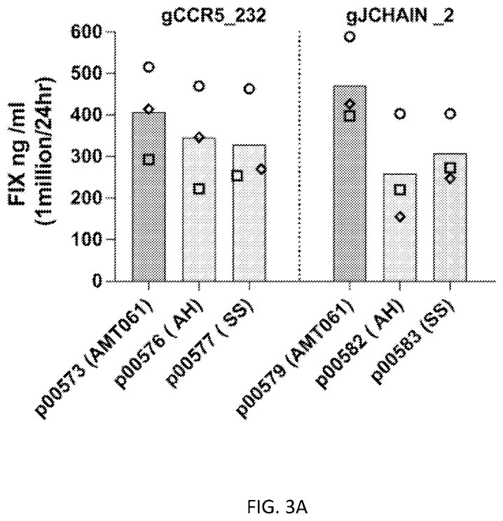

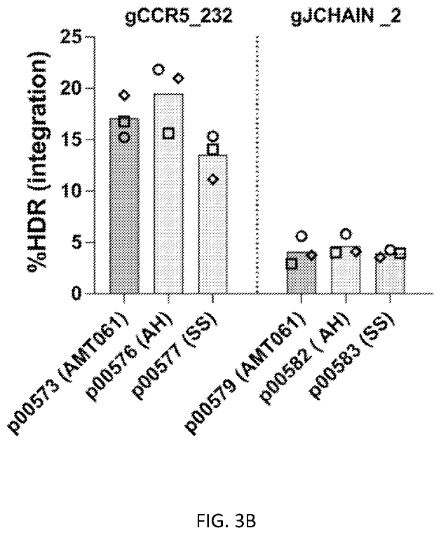

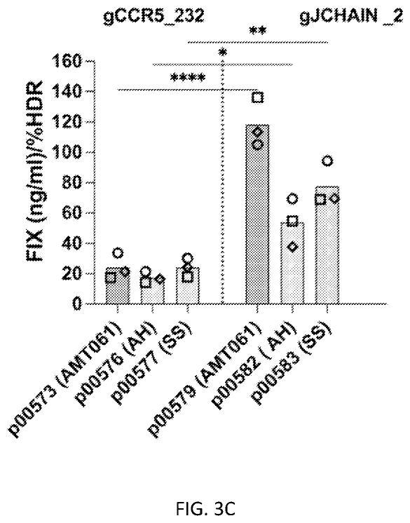

B lineage cells were engineered with human factor IX expression cassettes (hFIX) as outlined in FIG. 1 with either gCCR5_232 or gJCHAIN_2, expanded, and differentiated as described above. On day 13, an aliquot of 1×10⁶/ml B lineage cells were cultured for an additional 24 hours to measure hFIX and immunoglobulin G production using the ELISA methods described in Example 1 (FIGS. 2A, 2B, and 3A).

The assays were performed at least two times over three days to develop a standard curve and quality control samples (Low=3×Lower Limit of Detection (LLOD), Med=LLOD×UpperLimit of Detection (ULOD)*0.5 and High=75% of ULOD). IgG secretion rates were assessed as a measure of plasma cell function as well as the quality of the cell's secretory capacity.

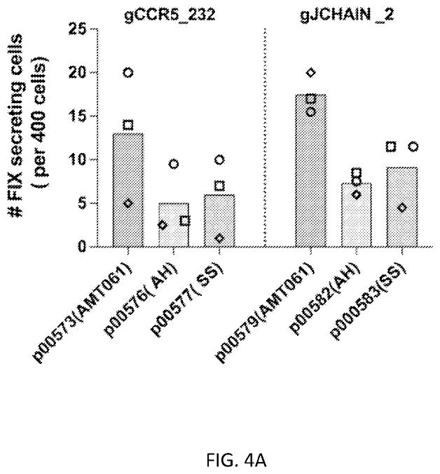

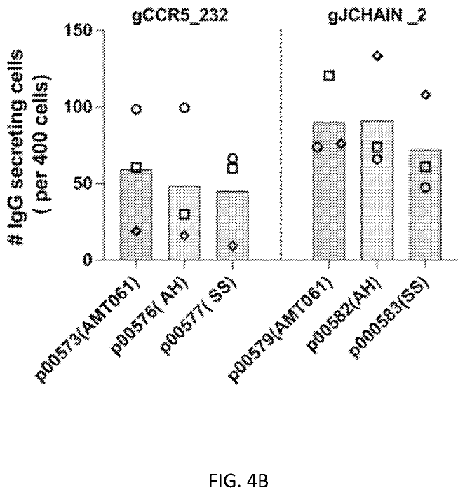

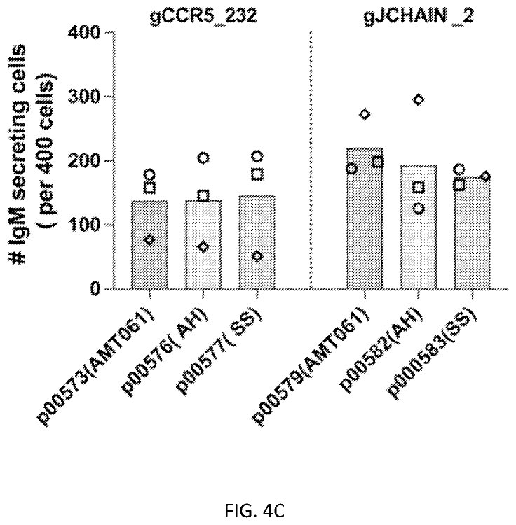

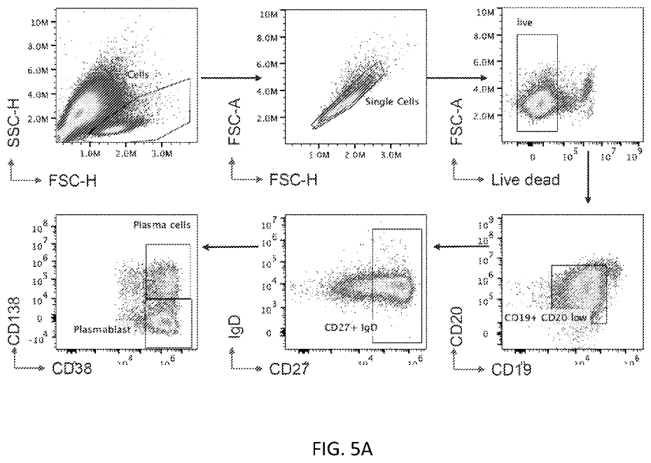

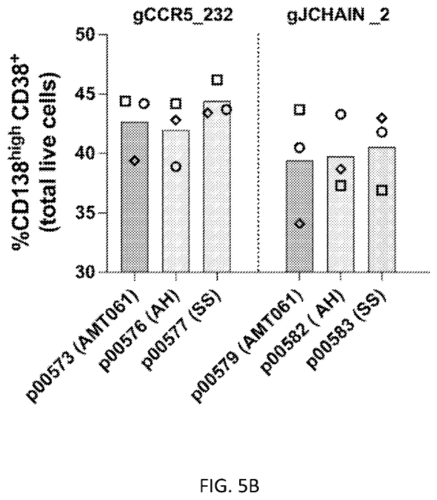

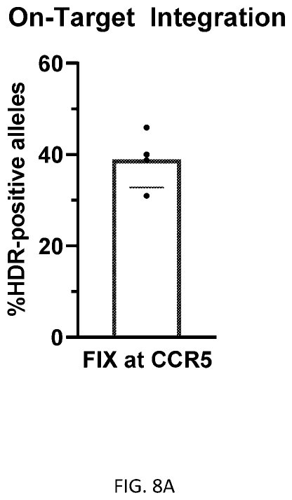

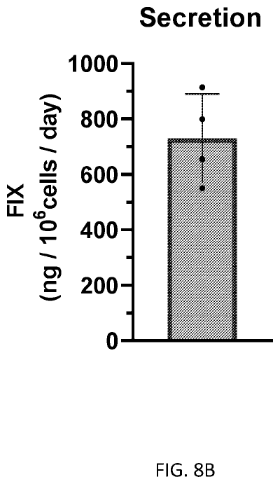

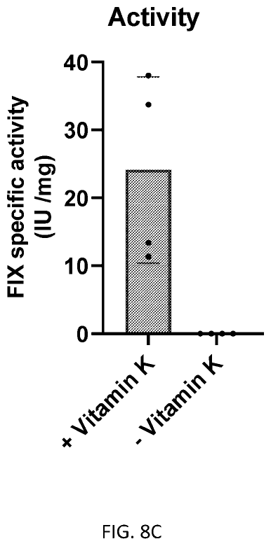

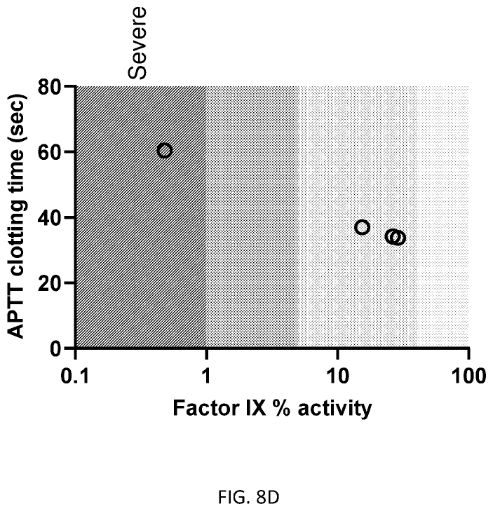

FIX transgene insertion frequency was assessed in the engineered B lineage cell populations by using the droplet digital polymerase chain reaction (ddPCR) method as described above in Example 1 (FIGS. 3B-C, and FIG. 8A). ELISpot as described above in Example 1 was performed on the engineered B lincage cells to measure FIX (FIGS. 4A and 8B), IgG (FIG. 4B), and IgM (FIG. 4C) secretion. Further, flow cytometry and cell count and viability as described in Example 1 were performed on the engineered B lineage cell populations to gate for viable plasma cell content (CD138^{high}CD38⁺) (FIGS. 5A-B).

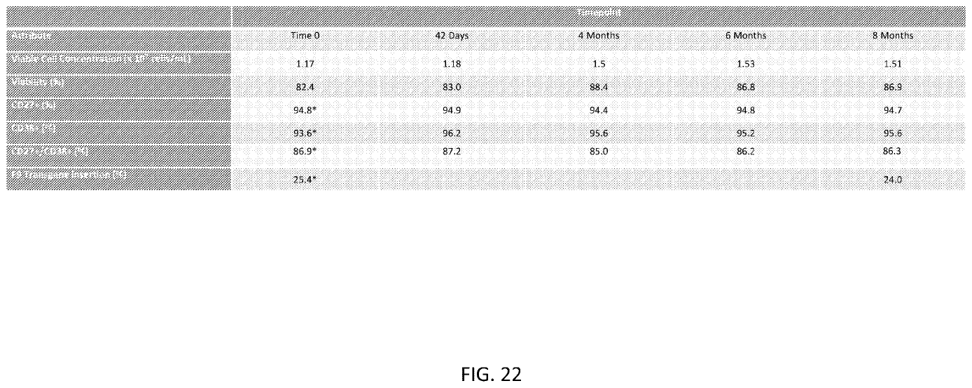

Additional B lineage cell population was isolated, activated, engineered with human Factor IX expression cassette, expanded, and differentiated as described herein. The engineered B lineage cell population were then cryopreserved and thawed as described herein. Viability and cell concentration was measured using acridine orange (AO)/DAPI staining, whereas percentage of CD27, CD38, and CD27/CD38 expression was measured via flow cytometry at various time points before (Time 0) and after 42 days, 4 months, 6 months, and 8 months of cryopreservation (FIG. 22).

Among other things, the present disclosure demonstrates that B lineage cell populations described herein can be engineered to express and/or secrete one or more transgenes of interest (e.g., FIX). In some embodiments, engineered B lineage cell populations may express and/or secrete FIX while maintaining comparable levels of IgG, as compared to a reference (e.g., non-engineered B lineage cell population, alternative engineered B lineage cell population, etc.). In some embodiments, engineered B lineage cell populations described herein may comprise increased plasma cell content when compared to a reference condition. In some embodiments, engineered B lineage cell populations using the engineering methods described herein may have transgene integration (e.g., percent HDR, FIX expression, etc.). In some embodiments, engineered B lineage cell populations as described herein may produce and secrete a product (e.g., polypeptide, protein, cytokine, etc.) encoded by the transgene (e.g., FIX). In some embodiments, culturing and engineering of B lineage cells as described herein may result in significant plasma cell content (>20% of total B lineage cell population). In some embodiments, culturing and engineering of B lineage cells as described herein may persist for at least about 8 months upon use of cooling methods.

### Example 3: Engineered B Lineage Cell Populations can Express and/or Secrete Functional Protein

The present Example demonstrates that engineered B lineage cell populations can express and/or secrete functional protein (e.g., FIX).

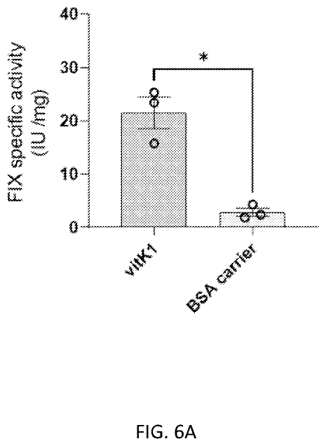

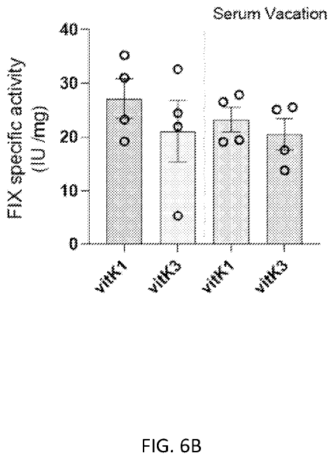

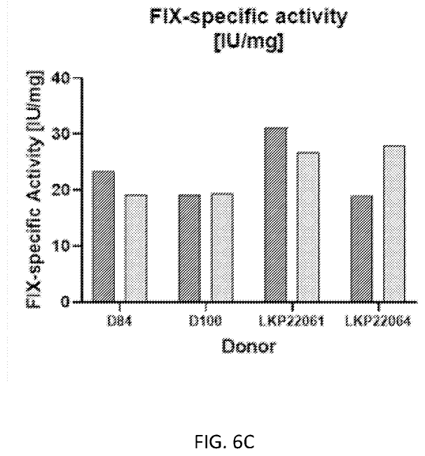

B lineage cells were isolated from different donors, activated, engineered with the hFIX expression cassette, expanded, and differentiated as described above. Engineered B lineage cells underwent FIX immunocapture activity assay as described in Example 1. Various forms of vitamin K were added into the culture media with the engineered B lineage cells. Initially, insoluble vitamin K1 (vitK1) was compared to BSA carrier control in terms of instigating FIX specific activity (FIG. 6A). Next, insoluble vitamin K1 was compared to soluble vitamin K3 (vitK3, Menadione Sodium Bisulfate). Culturing conditions as described above were compared to culturing conditions with absence of FBS for 24 hours post editing (serum variation) (FIG. 6B-C).

Furthermore, addition or absence of soluble vitamin K1 in culture media was tested with engineered B lineage cell populations described above using a FIX immunocapture activity assay as described supra (FIG. 8C). Furthermore, these engineered B lineage cell populations that were in culture media with or without soluble vitamin K were tested for their clotting activity using an activated partial thromboplastin clotting time (aPPT) assay as described in Example 1 (FIG. 8D).

Among other things, the present disclosure demonstrates that engineered B lineage cell populations described herein can express and/or secrete a functional protein (e.g., FIX). In some embodiments, engineered B lineage cell populations can express and/or secrete a functional protein with activity comparable to that of a reference condition (e.g., cell populations expressing endogenous, functional protein).

### Example 4: Evaluation of Engraftment of Engineered B Lineage Cell Populations

The present Example demonstrates that engineered B lineage cell populations may demonstrate engraftment and/or transgene expression (e.g., FIX). Subjects treated with engineered B lineage cell populations described herein may demonstrate persistent levels of transgene expression over an extended period of time (e.g., one or more weeks, one or more months, etc.).

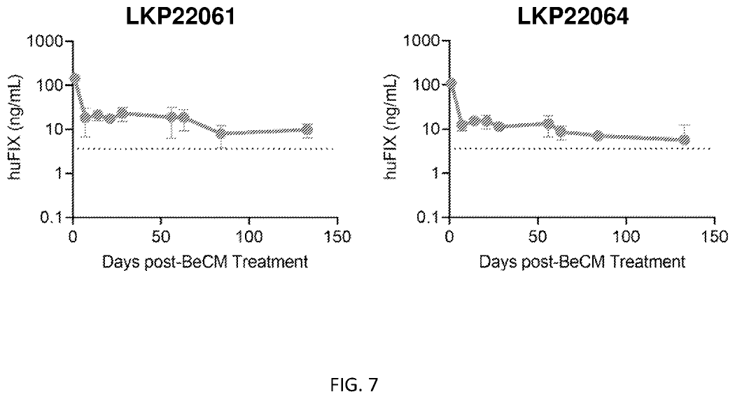

B lineages cells were isolated from two different donors (LKP22061 and LKP22064), activated, engineered with the hFIX expression cassette, expanded, and differentiated as described above. Engineered B lineage cells were administered into immunodeficient mouse model (NOG-hIL6 mouse). Mouse plasma samples were collected at 1, 7, 14, 28, and up to 150 days post-dosing, and assess for production of human immunoglobulin G (hIgG), human immunoglobulin M (hIgM), and hFIX by use of the ELISpot methods described above in Example 1 (FIG. 7).

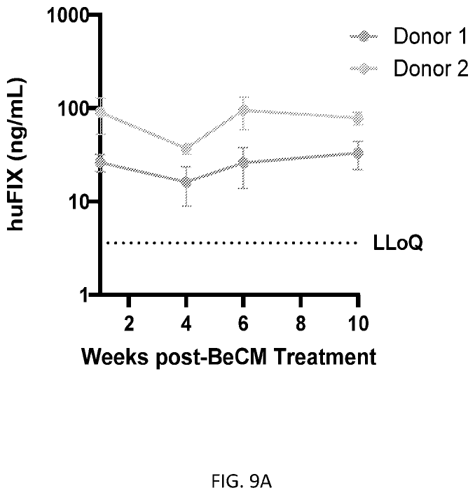

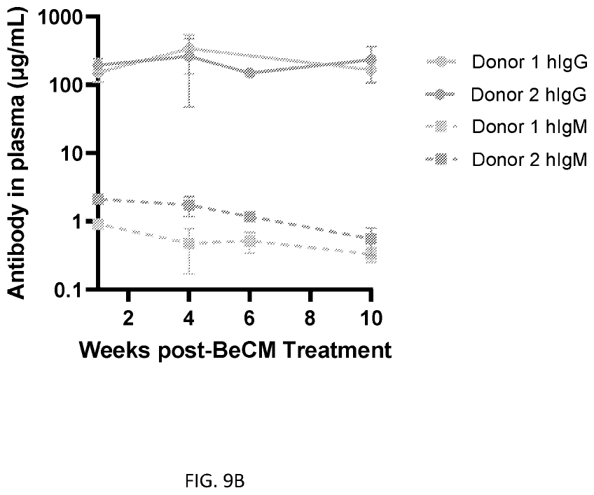

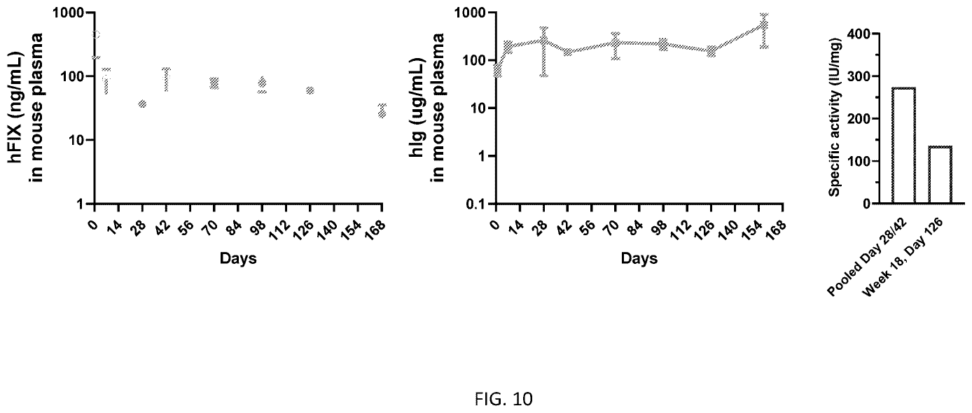

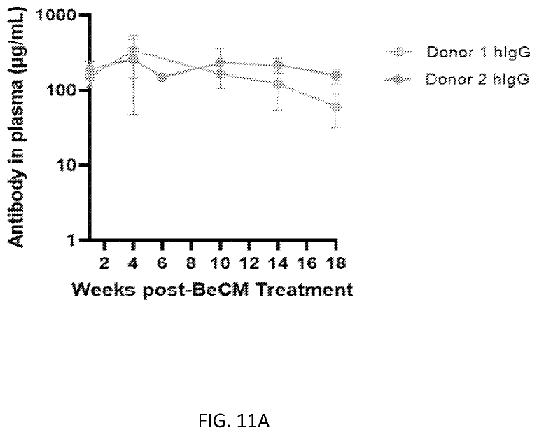

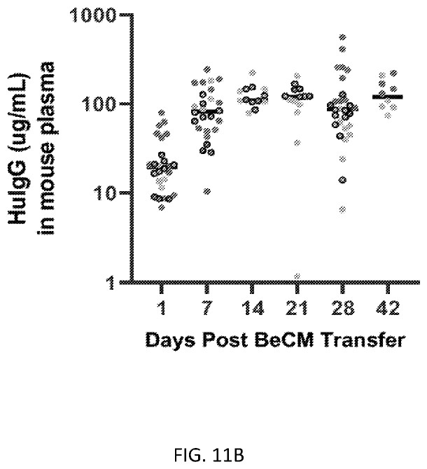

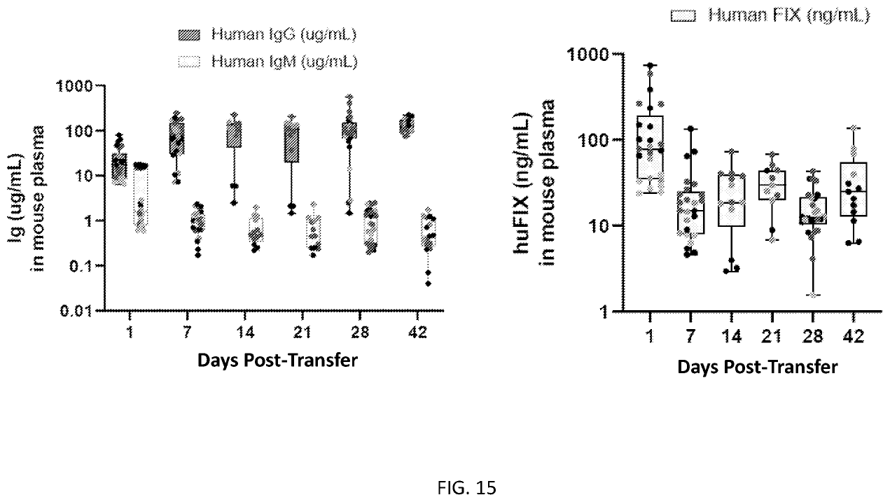

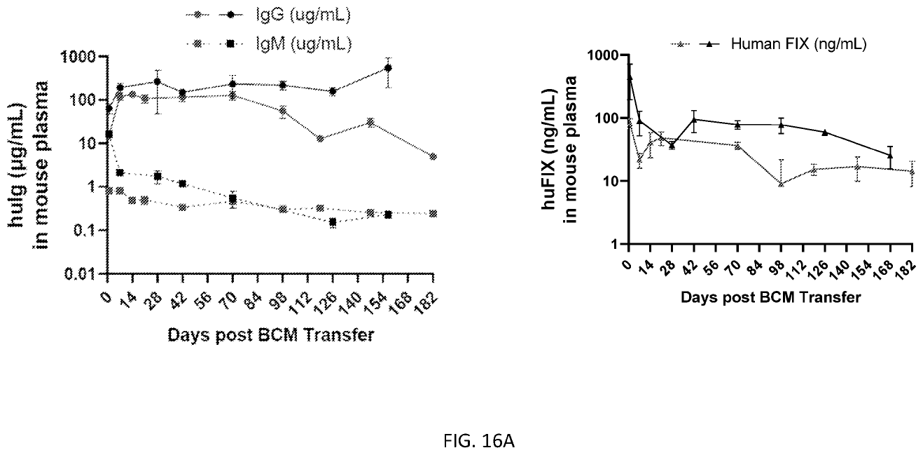

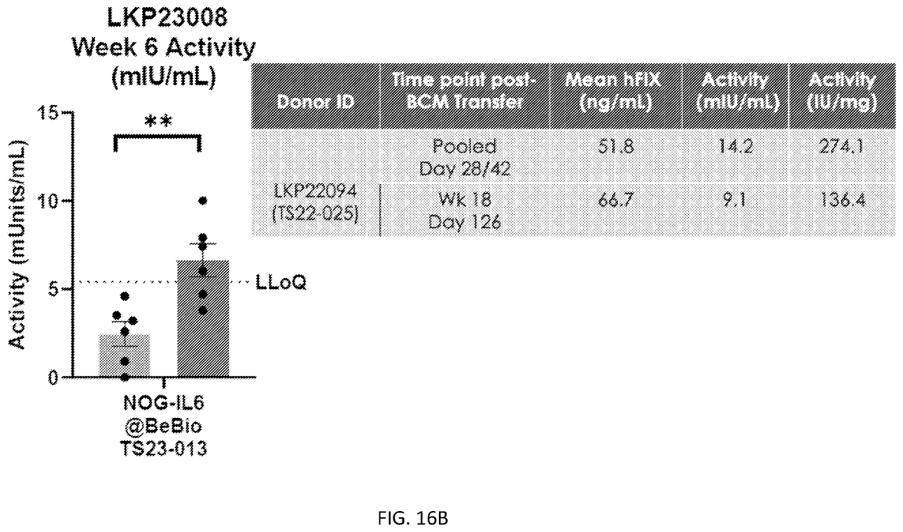

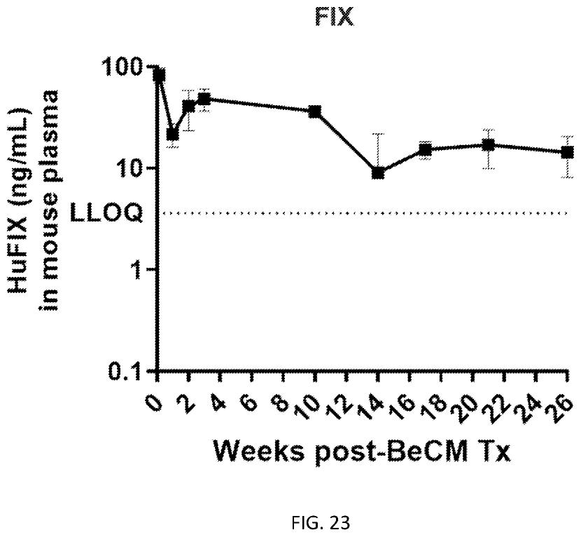

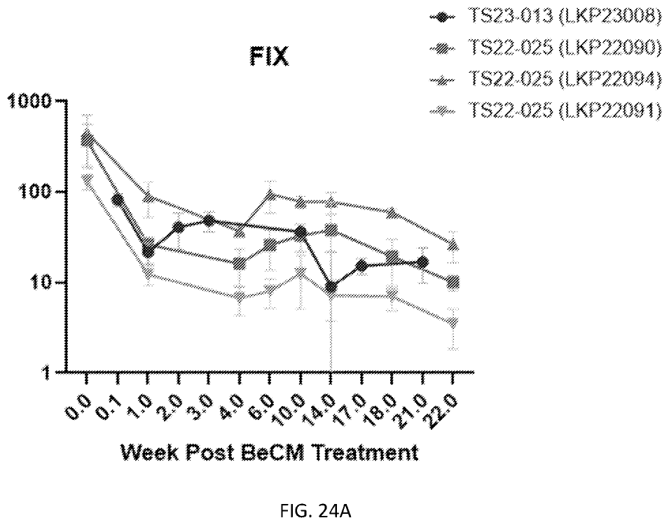

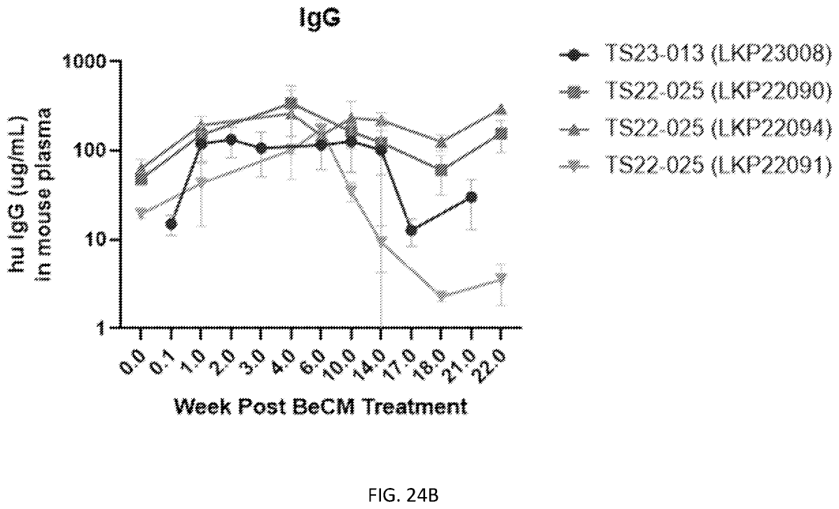

Additionally, B lineage cells were isolated from two different donors (Donor 1 and Donor 2), activated, engineered with an hFIX expression cassette, expanded, and differentiated as described above. Engineered B lineage cells were administered to a NOG-hIL6 mouse model, and mouse plasma samples were collected at 2, 4, 6, 8, and 10 weeks post-dosing. Mouse plasma samples collected from these two donors were assessed for hFIX (interchangeably, “huFIX”, FIG. 9A) as well as hIgG and hIgM (FIG. 9B). Additional experiments demonstrated hFIX expression and hIgG production at various timepoints post-treatment with engineered B lineage cells (FIG. 10, FIG. 11A, FIG. 11B, FIG. 15, FIG. 23, FIG. 24A, and FIG. 24B). Mice were also treated with samples that were either freshly engineered plasma cell populations or cryopreserved engineered plasma cell populations (FIG. 16A). Additional experiments were also performed (e.g., immunocapture two-stage chromogenic activity assay) to assess activity of hFIX in serum samples from treated mice (FIG. 16B).

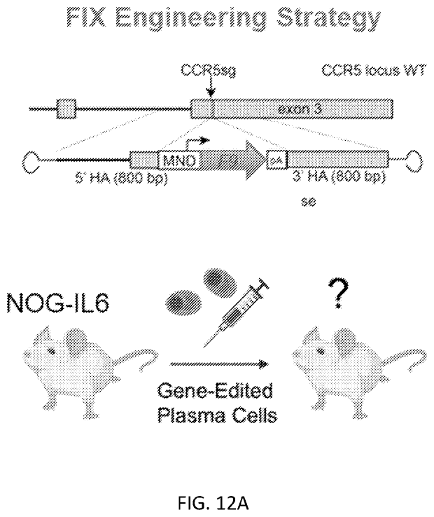

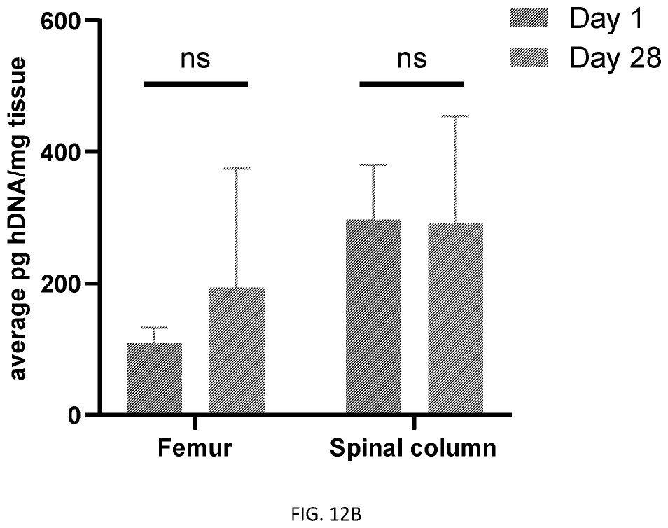

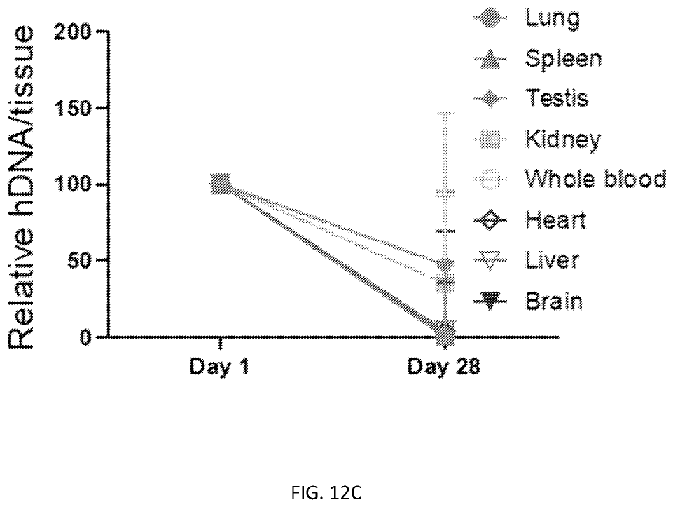

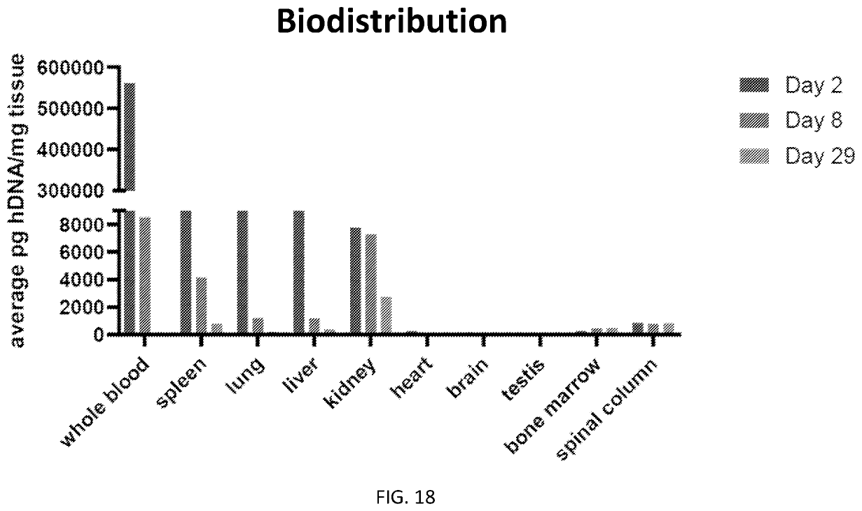

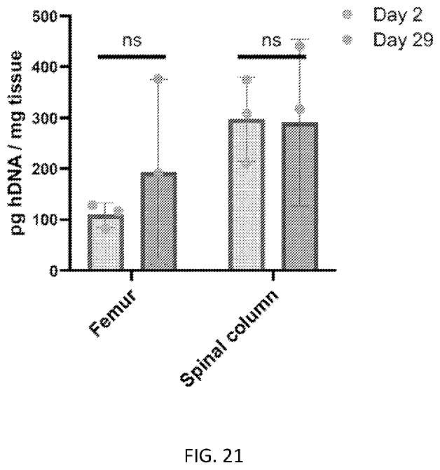

Samples of plasma from mice treated with plasma cell populations engineered to express hFIX were also assessed for persistence of integrated DNA over time. Specifically, integrated DNA levels were assessed in bone marrow (e.g., femur, spinal column) as compared to other tissues (FIG. 12A, FIG. 12B, FIG. 12C, FIG. 18, FIG. 21).

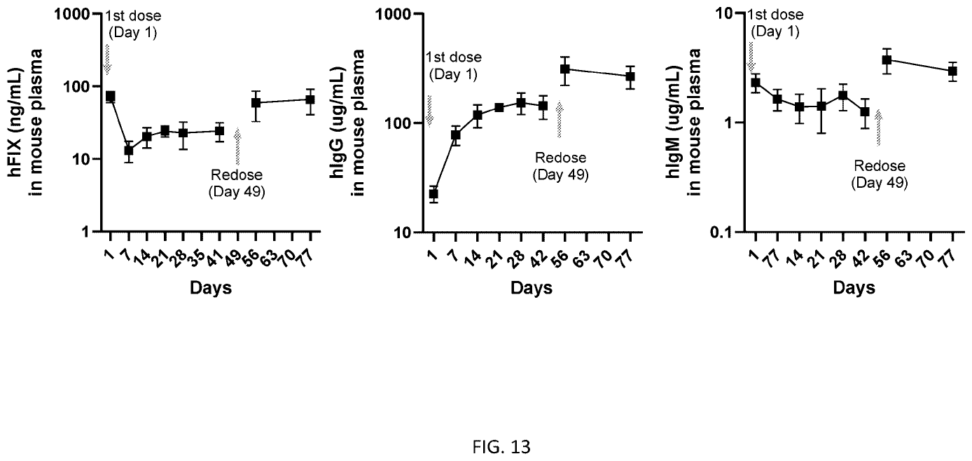

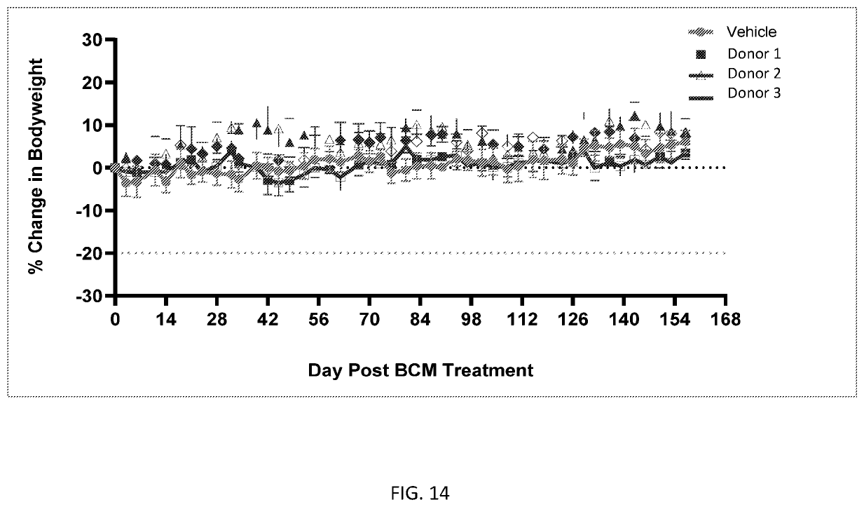

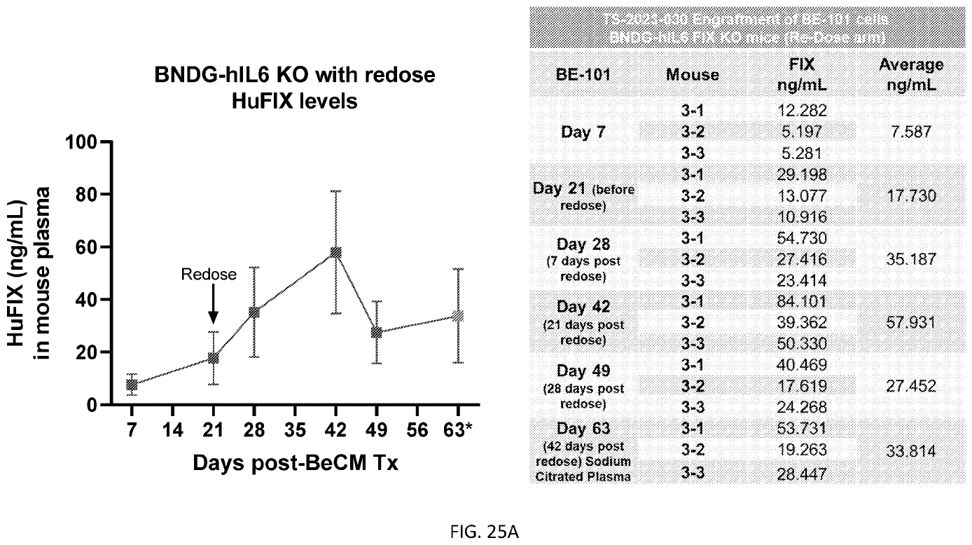

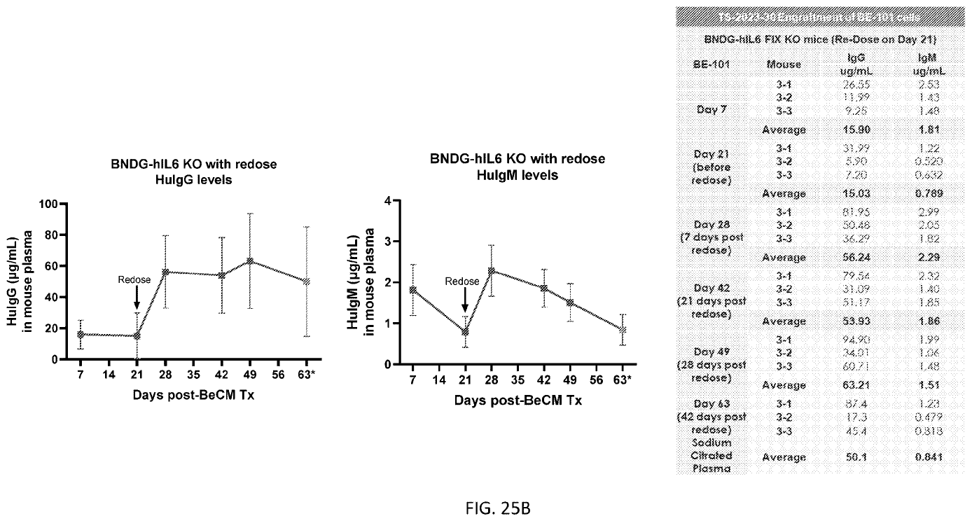

Mice treated with plasma cell populations engineered to express hFIX were also assessed after multiple doses. As shown in FIG. 13, an initial dosage of engineered plasma cells (20E6 cells/animal) was administered on day 1 of treatment, with a second dose (redoes, 20E6 cells/animal) on day 49. Percentage change in mouse body weight was also measured over an extended time period after administration of engineered plasma cell populations (FIG. 14). Additional in vivo experiments were undertaken involving an initial dosage of engineered plasma cells on day 1 of treatment with a second dose (Redose) occurring on day 21. Mouse serum was collected and HuFIX, IgG, and IgM was measured using methods disclosed in Example 1 (FIGS. 25A-B).

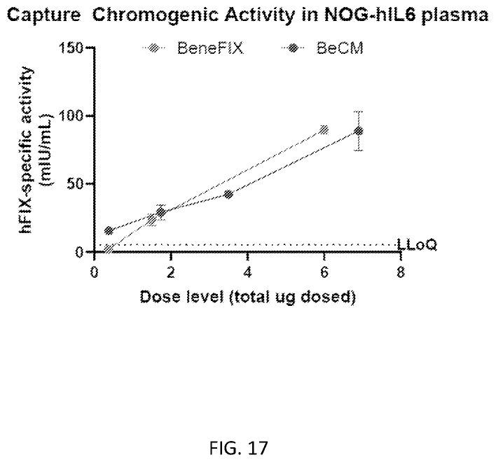

Additionally, mice were treated for one day with hFIX isolated from engineered plasma cell populations and a commercial FIX (BeneFIX®). Specific enzyme activity was compared at various doses, as shown in FIG. 17.

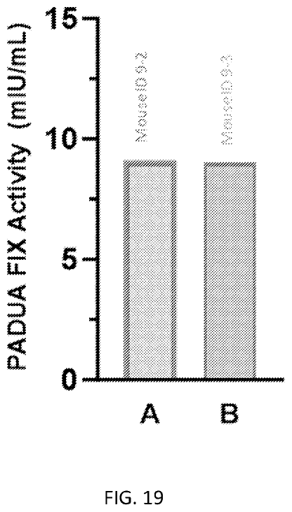

Mice were also treated with B lineage cell populations engineered to express FIX. Samples were taken at various timepoints, including day 126 after treatment and a pooled sample of day 98 and day 126, in order to measure FIX activity (FIG. 19).

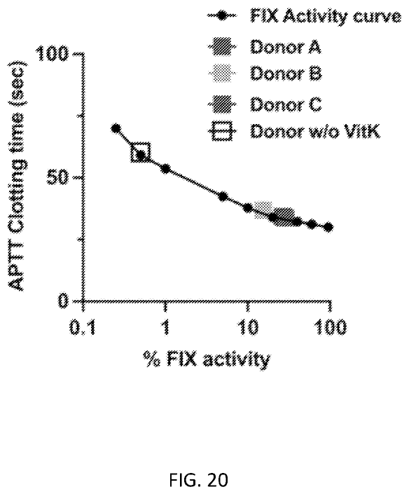

NOG-IL6 mice were also treated with plasma cell populations engineered to express FIX, and mouse plasma was collected to isolate and purify expressed FIX. FIX isolated and purified from the supernatant of engineered B lineage cell populations in the presence of Vitamin K versus a control lacking Vitamin K was assessed for activity using 1-stage aPTT assay (FIG. 20).

Among other things, the present disclosure demonstrates that engineered B lineage cell populations may provide engraftment and/or transgene expression (e.g., FIX) that is comparable or not significantly reduced as compared to a reference condition after administration to a subject. In some embodiments, engineered B lineage cell populations described herein may provide improved engraftment and/or transgene expression as compared to a reference condition. In some embodiments, engineered B lineage cell populations described herein may engraft and/or express transgene for extended durations (e.g., >12 weeks). In some embodiments, engineered B lineage cell populations described herein may engraft and persist long-term in a subject's bone marrow. In some embodiments, engineered B lineage cell populations described herein may exhibit Factor IX activity. In some embodiments, subjects may be treated with multiple doses of engineered B lineage cell populations (e.g., contemporaneously or at separate time points).

### Exemplary Sequences

TABLE 1
Exemplary Homology Arms (HA)

| | | SEQ ID |
|---|---|---|
| Name | Nucleotide Sequence | NO |
| gCCR5_232 5′ | GGTGCTATAGAGCACAAGATTTTATTTGGTGAGATGGTGCTTTCATGAATTC | 10 |
| HA | CCCCAACAGAGCCAAGCTCTCCATCTAGTGGACAGGGAAGCTAGCAGCAAA | |
| | CCTTCCCTTCACTACAAAACTTCATTGCTTGGCCAAAAAGAGAGTTAATTCA | |
| | ATGTAGACATCTATGTAGGCAATTAAAAACCTATTGATGTATAAAACAGTT | |
| | TGCATTCATGGAGGGCAACTAAATACATTCTAGGACTTTATAAAAGATCAC | |
| | TTTTTATTTATGCACAGGGTGGAACAAGATGGATTATCAAGTGTCAAGTCCA | |
| | ATCTATGACATCAATTATTATACATCGGAGCCCTGCCAAAAAATCAATGTG | |
| | AAGCAAATCGCAGCCCGCCTCCTGCCTCCGCTCTACTCACTGGTGTTCATCT | |
| | TTGGTTTTGTGGGCAACATGCTGGTCATCCTCATCCTGATAAACTGCAAAAG | |
| | GCTGAAGAGCATGACTGACATCTACCTGCTCAACCTGGCCATCTCTGACCTG | |
| | TTTTTCCTTCTTACTGTCCCCTTCTGGGCTCACTATGCTGCCGCCCAGTGGGA | |
| | CTTTGGAAATACAATGTGTCAACTCTTGACA | |
| gJCHAIN_2 5′ | tacaaaatacagattttgtgagatatatacaaaatacagattttgtgaga | 11 |
| HA | tatatacaaaatacagattttgtgagatatatacaaaatacatattttgt | |
| | gagatatatacaaaataTATATTTAAATCTGGTAGCTTGGTTAATTAAGGAC | |
| | TAGTATAAAAAATTAACATAAGATACACTTGGGACCTTAAAGGACTTTCAT | |
| | TTCTCTCCCCTTCCTTCATCTCTCCTCCTTCACTTACCTTCATTTATTATTCTA | |
| | GGCATGACAAGTGTCAGATGTCTGATTTGAAGACTAATATGAATATATCTA | |
| | GGTAATAAAATGGAAAATCCCTATTTCAACATTCTTAATGGAATCCTTCCTT | |
| | GAAATTATTATTAGTCTTCTGGTTTAAACATTTATATAGTGTAAATGCAAAA | |
| | TAAAAATGATTGTATCAAATTATGGTTTTTATTTGGATGCTTTGCAAGAAAA | |
| | TACTTAAATGTGAGTCATACATTTTTCCAGTTCAGCATTTAAACTTCAGACT | |
| | TCAACCACAGTTGTGATTGTTTTTAGTTTGTTAGCTGCCTGGAGTGTTATTTT | |
| | AAGAAAGCAGAAGCACCATCATTTGCACACT | |
| gJCHAIN_31 5′ | atacatattttgtgagatatatacaaaataTATATTTAAATCTGGTAGCTTGGTT | 12 |
| HA | AATTAAGGACTAGTATAAAAAATTAACATAAGATACACTTGGGACCTTAAAGGACT | |
| | TTCATTTCTCTCCCCTTCCTTCATCTCTCCTCCTTCACTTACCTTCATTTATTATT | |
| | CTAGGCATGACAAGTGTCAGATGTCTGATTTGAAGACTAATATGAATATATCTA | |
| | GGTAATAAAATGGAAAATCCCTATTTCAACATTCTTAATGGAATCCTTCCTT | |
| | GAAATTATTATTAGTCTTCTGGTTTAAACATTTATATAGTGTAAATGCAAAA | |
| | TAAAAATGATTGTATCAAATTATGGTTTTTATTTGGATGCTTTGCAAGAAAA | |
| | TACTTAAATGTGAGTCATACATTTTTCCAGTTCAGCATTTAAACTTCAGACT | |
| | TCAACCACAGTTGTGATTGTTTTTAGTTTGTTAGCTGCCTGGAGTGTTATTTT | |
| | AAGAAAGCAGAAGCACCATCATTTGCACACTCCTTATAGATCACACACCTT | |
| | AACCCTGACTTTTTTTGCTCCAGTTTTTCAGAAGAAGTGAAGTCAAGATGAA | |
| | GAACCATTTGCTTTT | |
| gJCHAIN_54 5′ | TTTTCTAAAGAAAAAATCATTGGCATTCATAGAAAGGCCAATTTCTCTTAAT | 13 |
| HA | AGTTCAATAAGTGGATTTGATCTTATAAAAAGGCAGGTGTTTCTTTGGAAAT | |
| | GACAGACTCCAACATCAATTTTTTTAAAAATTCTCCCTTTCTTGTCACTATA | |
| | AATAACTTGTTTAGACAGATATACAGTTGGGAATAAGCCTAACACAGTAGA | |
| | AATTGCTGTATGGTGTAGATAAAACAATCATATTATCATATCATTAATTATA | |
| | TTGCTTACTTTCAACTAATATATATTAAAGATTGGAAAATCCCATAAGCTAT | |
| | TCTGTATTGTAGAGCTGCTTATGTCTGAAAGGAGTCATCCCTTGCTGTCATG | |
| | TCAGAGCTGCAAGAACTAATTGATTTTGGATTGAAATGTGTAGTCACATTTT | |
| | GAGACAGCATTTGAGGGGATTGTCTAATACATATATTTGCTTTTCAGCTGTA | |
| | AAAAATGTGATCCTACAGAAGTGGAGCTGGATAATCAGATAGTTACTGCTA | |
| | CCCAGAGCAATATCTGTGATGAAGACAGTGCTACAGAGACCTGCTACACTT | |
| | ATGACAGAAACAAGTGCTACACAGCTGTGGT | |
| gJtolGHM_23 5′ | ATGTGACGCCCGGAGACAGAAGGTCTCTGGGTGGCTGGGTTTTTGTGGGGT | 14 |
| HA | GAGGATGGACATTCTGCCATTGTGATTACTACTACTACTACTACATGGACGT | |
| | CTGGGGCAAAGGGACCACGGTCACCGTCTCCTCAGGTAAGAATGGCCACTC | |
| | TAGGGCCTTTGTTTTCTGCTACTGCCTGTGGGGTTTCCTGAGCATTGCAGGT | |
| | TGGTCCTCGGGGCATGTTCCGAGGGGACCTGGGCGGACTGGCCAGGAGGGG | |
| | ATGGGCACTGGGGTGCCTTGAGGATCTGGGAGCCTCTGTGGATTTTCCGAT | |
| | GCCTTTGGAAAATGGGACTCAGGTTGGGTGCGTCTGATGGAGTAACTGAGC | |
| | CTGGGGGCTTGGGGAGCCACATTTGGACGAGATGCCTGAACAAACCAGGG | |
| | GTCTTAGTGATGGCTGAGGAATGTGTCTCAGGAGCGGTGTC | |
| gCCR5_232 3′ | CTCTATTTTATAGGCTTCTTCTCTGGAATCTTCTTCATCATCCTCCTGACAAT | 15 |
| HA | CGATAGGTACCTGGCTGTCGTCCATGCTGTGTTTGCTTTAAAAGCCAGGACG | |
| | GTCACCTTTGGGGTGGTGACAAGTGTGATCACTTGGGTGGTGGCTGTGTTTG | |
| | CGTCTCTCCCAGGAATCATCTTTACCAGATCTCAAAAAGAAGGTCTTCATTA | |
| | CACCTGCAGCTCTCATTTTCCATACAGTCAGTATCAATTCTGGAAGAATTTC | |
| | CAGACATTAAAGATAGTCATCTTGGGGCTGGTCCTGCCGCTGCTTGTCATGG | |
| | TCATCTGCTACTCGGGAATCCTAAAAACTCTGCTTCGGTGTCGAAATGAGA | |
| | AGAAGAGGCACAGGGCTGTGAGGCTTATCTTCACCATCATGATTGTTTATTT | |
| | TCTCTTCTGGGCTCCCTACAACATTGTCCTTCTCCTGAACACCTTCCAGGAA | |
| | TTCTTTGGCCTGAATAATTGCAGTAGCTCTAACAGGTTGGACCAAGCTATGC | |
| | AGGTGACAGAGACTCTTGGGATGACGCACTGCTGCATCAACCCCATCATCT | |
| | ATGCCTTTGTCGGGGAGAAGTTCAGAAAC | |
| gJCHAIN_2 3′ | AGATCACACACCTTAACCCTGACTTTTTTTGCTCCAGTTTTTCAGAAGAAGT | 16 |
| HA | GAAGTCAAGATGAAGAACCATTTGCTTTTCTGGGGAGTCCTGGCGGTTTTTA | |
| | TTAAGGCTGTTCATGTGAAAGGTATGTGATATTTAGAAAATGATCCCAGAT | |
| | CAAAGGAAAAATATAGGACAGATCTGTTTTTCAGTTTTAAACATTTATGCCT | |
| | ATGCTTTGGTTGGCCAGTTAGCCAGCTACTTTTTCCTAACATGCTTTCATCTT | |
| | TCTATGGCAGTTGTCTTTACATAGATAAGACTCTTTTTATTCTTTTTTCTCTT | |
| | ATCTTACTTACTTGTTTGCCTACTACAAACTAGAGAATAAGAAATTAGCTAA | |
| | CTACAGATAGTCCTGCTTATATGAATGTCCATATTGAATCTTAAAAATCTAT | |
| | AATCCTTCCCTTGAGTCTCTTAAATTAATATTCTGGAGAAATGAAATAATAA | |
| | TTACTGTAAGTACAACATGGAATGCATTGGGATTTTATTTCCCCAAAGAGGC | |
| | TGGTATTGGTTTTAACAGCTTTCCTGGATCTGATCCCACAGATTTATTATGA | |
| | CCCTTTAACAATGTTTAATGTTATACA | |
| gJCHAIN_54 3′ | CGTATATGGTGGTGAGACCAAAATGGTGGAAACAGCCTTAACCCCAGATGC | 17 |
| HA | CTGCTATCCTGACTAATTTAAGTCATTGCTGACTGCATAGCTCTTTTTCTTGA | |
| | GAGGCTCTCCATTTTGATTCAGAAAGTTAGCATATTTATTACCAATGAATTTGAAA | |
| | CCAGGGCttttttttttttttGGGTGATGTAAAACCAACTCCCTGCCACCAAAAT | |
| | AATTAAAATAGTCACATTGTTATCTTTATTAGGTAATCACTTCTTAATTATAT | |
| | GTTCATACTCTAAGTATCAAAATCTTCCAATTATCATGCTCACCTGAAAGAG | |
| | GTATGCTCTCTTAGGAATACAGTTTCTAGCATTAAACAAATAAACAAGGGG | |
| | AGAAAATAAAACTCAAGGACTGAAAATCAGGAGGTGTAATAAAATGTTCCTCGCATT | |
| | CCCCCCCGCttttttttttttttttGACTTTGCCTTGGAGAGCCAGAGCTTCCGC | |
| | ATTTTCTTTACTATTCTTTTTAAAAAAAGTTTCACTGTGTAGAGAACATATAT | |
| | GCATAAACATAGGTCAATTATATGTCTCCATTAGAAAAATAATAATTGGAA | |
| | AACATGTTCTAGA | |
| gJtolGHM_23 3′ | TGTAGGACTGCAAGATCGCTGCACAGCAGCGAATCGTGAAATATTTTCTTT | 18 |
| HA | AGAATTATGAGGTGCGCTGTGTGTCAACCTGCATCTTAAATTCTTTATTGGC | |
| | TGGAAAGAGAACTGTCGGAGTGGGTGAATCCAGCCAGGAGGGACGCGTAG | |
| | CCCCGGTCTTGATGAGAGCAGGGTTGGGGGCAGGGGTAGCCCAGAAACGG | |
| | TGGCTGCCGTCCTGACAGGGGCTTAGGGAGGCTCCAGGACCTCAGTGCCTT | |
| | GAAGCTGGTTTCCATGAGAAAAGGATTGTTTATCTTAGGAGGCATGCTTACT | |
| | GTTAAAAGACAGGATATGTTTGAAGTGGCTTCTGAGAAAAATGGTTAAGAA | |
| | AATTATGACTTAAAAATGTGAGAGATTTTCAAGTATATTAATTTTTTTAACT | |
| | GTCCAAGTATTTGAAATTCTTATCATTTGATTAACACCCAT | |

TABLE 2
Exemplary Expression Cassettes

| | | SEQ ID |
|---|---|---|
| Name | Nucleotide Sequence | NO |
| ATM061 | GCCACCATGCAGAGGGTGAACATGATCATGGCTGAGAGCCCTGGCCTGAT | 19 |
| | CACCATCTGCCTGCTGGGCTACCTGCTGTCTGCTGAGTGCACTGTGTTCCT | |
| | GGACCATGAGAATGCCAACAAGATCCTGAACAGGCCCAAGAGATACAAC | |
| | TCTGGCAAGCTGGAGGAGTTTGTGCAGGGCAACCTGGAGAGGGAGTGCA | |
| | TGGAGGAGAAGTGCAGCTTTGAGGAGGCCAGGGAGGTGTTTGAGAACAC | |
| | TGAGAGGACCACTGAGTTCTGGAAGCAGTATGTGGATGGGGACCAGTGT | |
| | GAGAGCAACCCCTGCCTGAATGGGGGCAGCTGCAAGGATGACATCAACA | |
| | GCTATGAGTGCTGGTGCCCCTTTGGCTTTGAGGGCAAGAACTGTGAGCTG | |
| | GATGTGACCTGCAACATCAAGAATGGCAGATGTGAGCAGTTCTGCAAGA | |
| | ACTCTGCTGACAACAAGGTGGTGTGCAGCTGCACTGAGGGCTACAGGCTG | |
| | GCTGAGAACCAGAAGAGCTGTGAGCCTGCTGTGCCATTCCCATGTGGCAG | |
| | AGTGTCTGTGAGCCAGACCAGCAAGCTGACCAGGGCTGAGGCTGTGTTCC | |
| | CTGATGTGGACTATGTGAACAGCACTGAGGCTGAAACCATCCTGGACAAC | |
| | ATCACCCAGAGCACCCAGAGCTTCAATGACTTCACCAGGGTGGTGGGGG | |
| | GGGAGGATGCCAAGCCTGGCCAGTTCCCCTGGCAAGTGGTGCTGAATGGC | |
| | AAGGTGGATGCCTTCTGTGGGGGCAGCATTGTGAATGAGAAGTGGATTGT | |
| | GACTGCTGCCCACTGTGTGGAGACTGGGGTGAAGATCACTGTGGTGGCTG | |
| | GGGAGCACAACATTGAGGAGACTGAGCACACTGAGCAGAAGAGGAATGT | |
| | GATCAGGATCATCCCCCACCACAACTACAATGCTGCCATCAACAAGTACA | |
| | ACCATGACATTGCCCTGCTGGAGCTGGATGAGCCCCTGGTGCTGAACAGC | |
| | TATGTGACCCCCATCTGCATTGCTGACAAGGAGTACACCAACATCTTCCT | |
| | GAAGTTTGGCTCTGGCTATGTGTCTGGCTGGGGCAGGGTGTTCCACAAGG | |
| | GCAGGTCTGCCCTGGTGCTGCAGTACCTGAGGGTGCCCCTGGTGGACAGG | |
| | GCCACCTGCCTGCTGAGCACCAAGTTCACCATCTACAACAACATGTTCTG | |
| | TGCTGGCTTCCATGAGGGGGGCAGGGACAGCTGCCAGGGGGACTCTGGG | |
| | GGCCCCCATGTGACTGAGGTGGAGGGCACCAGCTTCCTGACTGGCATCAT | |
| | CAGCTGGGGGGAGGAGTGTGCCATGAAGGGCAAGTATGGCATCTACACC | |
| | AAAGTCTCCAGATATGTGAACTGGATCAAGGAGAAGACCAAGCTGACCT | |
| | GA | |
| NO CG SS | GCCACCATGCAGAGGGTGAACATGATCATGGCTGAGAGCCCTGGCCTGAT | 20 |
| | CACCATCTGCCTGCTGGGCTACCTGCTGAGTGCTGAGTGCACAGTGTTCCT | |
| | GGACCATGAGAATGCCAACAAGATCCTGAACAGACCCAAGAGATACAAC | |
| | AGTGGCAAGCTGGAGGAGTTTGTGCAGGGCAACCTGGAGAGGGAGTGCA | |
| | TGGAGGAGAAGTGCAGCTTTGAGGAGGCCAGGGAAGTGTTTGAGAACAC | |
| | AGAGAGAACCACAGAGTTCTGGAAGCAGTATGTGGATGGAGACCAGTGT | |
| | GAGAGCAACCCTTGCCTGAATGGAGGCAGCTGCAAGGATGACATCAACA | |
| | GCTATGAGTGCTGGTGCCCTTTTGGCTTTGAGGGCAAGAACTGTGAGCTG | |
| | GATGTGACCTGCAACATCAAGAATGGAAGGTGTGAGCAGTTCTGCAAGA | |
| | ACTCTGCTGACAACAAAGTGGTGTGTAGCTGCACAGAGGGCTACAGACTG | |
| | GCTGAGAACCAGAAGTCTTGTGAGCCAGCTGTGCCTTTTCCCTGTGGCAG | |
| | AGTCAGTGTGTCCCAGACCAGCAAGCTGACCAGAGCTGAGACAGTGTTCC | |
| | CTGATGTGGACTATGTGAATAGCACAGAGGCTGAGACCATCCTGGACAAC | |
| | ATCACCCAGAGCACCCAGTCCTTCAATGACTTCACCAGAGTTGTGGGAGG | |
| | GGAGGATGCCAAGCCTGGCCAGTTCCCCTGGCAGGTGGTGCTGAATGGCA | |
| | AAGTGGATGCCTTCTGTGGAGGCAGCATTGTGAATGAGAAGTGGATTGTG | |
| | ACAGCTGCCCACTGTGTGGAGACAGGAGTGAAGATCACAGTGGTGGCTG | |
| | GAGAACACAATATTGAGGAGACAGAGCACACTGAGCAGAAGAGGAATGT | |
| | CATCAGAATTATCCCCCACCACAACTACAATGCTGCCATCAACAAGTACA | |
| | ACCATGACATTGCCCTGCTGGAGCTGGATGAGCCTCTGGTGCTGAATAGC | |
| | TATGTGACCCCCATCTGCATTGCTGACAAGGAGTACACCAACATCTTCCT | |
| | GAAGTTTGGCAGTGGCTATGTCAGTGGCTGGGGCAGAGTGTTCCACAAGG | |
| | GCAGATCTGCCCTGGTGCTGCAGTACCTGAGAGTGCCCCTGGTGGACAGA | |
| | GCCACCTGCCTGTTGAGCACCAAGTTCACCATCTACAACAACATGTTCTG | |
| | TGCTGGCTTCCATGAGGGAGGCAGAGACAGCTGCCAGGGAGATTCTGGA | |
| | GGGCCCCATGTGACAGAAGTGGAGGGCACCAGCTTCCTGACAGGCATCA | |
| | TCAGCTGGGGGGAGGAGTGTGCCATGAAGGGCAAGTATGGCATCTACAC | |
| | CAAAGTCAGCAGATATGTGAACTGGATCAAGGAGAAAACCAAGCTGACC | |
| | TGA | |
| NO CG AH | GCCACCATGCAGAGGGTGAACATGATCATGGCAGAGAGCCCTGGCCTGA | 21 |
| | TCACCATCTGCCTGCTGGGCTACCTGCTGTCTGCAGAATGTACAGTGTTTC | |
| | TGGACCATGAGAATGCCAACAAGATCCTGAACAGGCCCAAGAGATACAA | |
| | CTCTGGCAAGCTGGAGGAGTTTGTGCAGGGCAACCTGGAGAGGGAGTGC | |
| | ATGGAGGAGAAGTGCAGCTTTGAGGAGGCCAGGGAAGTGTTTGAGAACA | |
| | CTGAGAGGACCACTGAGTTCTGGAAGCAGTATGTGGATGGAGACCAGTG | |
| | TGAGAGCAACCCTTGCCTGAATGGAGGCAGCTGCAAGGATGACATCAAC | |
| | AGCTATGAGTGCTGGTGCCCATTTGGCTTTGAGGGCAAGAACTGTGAGCT | |
| | GGATGTGACCTGCAACATCAAGAATGGCAGATGTGAGCAGTTCTGCAAG | |
| | AACTCTGCTGACAACAAAGTGGTGTGTAGCTGCACTGAGGGCTACAGACT | |
| | GGCAGAGAATCAGAAGTCCTGTGAACCTGCTGTGCCTTTTCCCTGTGGCA | |
| | GAGTCTCTGTGTCCCAGACCAGCAAGCTGACCAGAGCAGAGACTGTGTTC | |
| | CCAGATGTGGACTATGTGAATAGCACTGAGGCAGAGACCATCCTGGACA | |
| | ACATCACCCAGAGCACCCAGTCCTTCAATGACTTCACCAGAGTTGTGGGA | |
| | GGAGAGGATGCCAAGCCAGGCCAGTTCCCCTGGCAGGTGGTGCTGAATG | |
| | GCAAAGTGGATGCCTTCTGTGGAGGCAGCATTGTGAATGAGAAGTGGATT | |
| | GTGACAGCAGCCCACTGTGTGGAGACTGGAGTGAAGATCACTGTGGTGG | |
| | CAGGAGAACACAATATTGAGGAGACTGAGCACACAGAGCAGAAGAGAA | |
| | ATGTCATCAGGATTATCCCCCACCACAACTACAATGCAGCCATCAACAAG | |
| | TACAACCATGACATTGCCCTGCTGGAGCTGGATGAGCCTCTGGTGCTGAA | |
| | TAGCTATGTGACCCCCATCTGCATTGCAGACAAGGAGTACACCAACATCT | |
| | TCCTGAAGTTTGGCTCTGGCTATGTCAGTGGCTGGGGCAGAGTGTTCCAC | |
| | AAGGGCAGATCTGCCCTGGTGCTGCAGTACCTGAGAGTGCCCCTGGTGGA | |
| | CAGAGCCACCTGCCTGTTGAGCACCAAGTTCACCATCTACAACAACATGT | |
| | TCTGTGCAGGCTTCCATGAGGGAGGCAGAGACAGCTGCCAGGGAGACTC | |
| | TGGAGGACCCCATGTGACTGAAGTGGAGGGCACCAGCTTCCTGACTGGCA | |
| | TCATCAGCTGGGGAGAGGAGTGTGCCATGAAGGGCAAGTATGGCATCTA | |
| | CACCAAAGTCAGCAGATATGTGAACTGGATCAAGGAGAAAACCAAGCTG | |
| | ACCTGA | |

TABLE 3
Exemplary Promoters

| | | SEQ |
|---|---|---|
| | | ID |
| Name | Nucleotide Sequence | NO |
| MND | gaacagttggaacagcagaatatgggccaaacagg | 22 |
| | atatctgtggtaagcagttcctgccccggctcagg | |
| | gccaagaacagttggaacagcagaatatgggccaa | |
| | acaggatatctgtggtaagcagttcctgccccggc | |
| | tcagggccaagaacagatggtccccagatgcggtc | |
| | ccgccctcagcagtttctagagaaccatcagatgt | |
| | ttccagggtgccccaaggacctgaaatgaccctgt | |
| | gccttatttgaactaaccaatcagttcgcttctcg | |
| | cttctgttcgcgcgcttctgctccccgagctctat | |
| | ataagcagagctcgtttagtgaaccgtcagatc | |

TABLE 4
Exemplary Transgene

| | | SEQ |
|---|---|---|
| Name | Nucleotide Sequence | ID NO |
| Factor | ATGCAGCGCGTGAACATGATCATGGCAGAATCACCAGGCCTCATCACCA | 23 |
| IX CDS | TCTGCCTTTTAGGATATCTACTCAGTGCTGAATGTACAGTTTTTCTTGATC | |
| | ATGAAAACGCCAACAAAATTCTGAATCGGCCAAAGAGGTATAATTCAGG | |
| | TAAATTGGAAGAGTTTGTTCAAGGGAACCTTGAGAGAGAATGTATGGAA | |
| | GAAAAGTGTAGTTTTGAAGAAGCACGAGAAGTTTTTGAAAACACTGAAA | |
| | GAACAACTGAATTTTGGAAGCAGTATGTTGATGTAACATGTAACATTAA | |
| | GAATGGCAGATGCGAGCAGTTTTGTAAAAATAGTGCTGATAACAAGGTG | |
| | GTTTGCTCCTGTACTGAGGGATATCGACTTGCAGAAAACCAGAAGTCCT | |
| | GTGAACCAGCAGTGCCATTTCCATGTGGAAGAGTTTCTGTTTCACAAACT | |
| | TCTAAGCTCACCCGTGCTGAGACTGTTTTTCCTGATGTGGACTATGTAAA | |
| | TTCTACTGAAGCTGAAACCATTTTGGATAACATCACTCAAAGCACCCAA | |
| | TCATTTAATGACTTCACTCGGGTTGTTGGTGGAGAAGATGCCAAACCAG | |
| | GTCAATTCCCTTGGCAGGTTGTTTTGAATGGTAAAGTTGATGCATTCTGT | |
| | GGAGGCTCTATCGTTAATGAAAAATGGATTGTAACTGCTGCCCACTGTG | |
| | TTGAAACTGGTGTTAAAATTACAGTTGTCGCAGGTGAACATAATATTGA | |
| | GGAGACAGAACATACAGAGCAAAAGCGAAATGTGATTCGAATTATTCCT | |
| | CACCACAACTACAATGCAGCTATTAATAAGTACAACCATGACATTGCCC | |
| | TTCTGGAACTGGACGAACCCTTAGTGCTAAACAGCTACGTTACACCTATT | |
| | TGCATTGCTGACAAGGAATACACGAACATCTTCCTCAAATTTGGATCTG | |
| | GCTATGTAAGTGGCTGGGGAAGAGTCTTCCACAAAGGGAGATCAGCTTT | |
| | AGTTCTTCAGTACCTTAGAGTTCCACTTGTTGACCGAGCCACATGTCTTC | |
| | GATCTACAAAGTTCACCATCTATAACAACATGTTCTGTGCTGGCTTCCAT | |
| | GAAGGAGGTAGAGATTCATGTCAAGGAGATAGTGGGGGACCCCATGTT | |
| | ACTGAAGTGGAAGGGACCAGTTTCTTAACTGGAATTATTAGCTGGGGTG | |
| | AAGAGTGTGCAATGAAAGGCAAATATGGAATATATACCAAGGTATCCCG | |
| | GTATGTCAACTGGATTAAGGAAAAAACAAAGCTCACTTAA | |

TABLE 5
Exemplary Terminator Sequences

| | | SEQ |
|---|---|---|
| Name | Nucleotide Sequence | ID NO |
| BGH | ctgtgccttctagttgccagccatctgttgtttgc | 24 |
| polyA | ccctcccccgtgccttccttgaccctggaaggtgc | |
| | cactcccactgtcctttcctaataaaatgaggaaa | |
| | ttgcatcgcattgtctgagtaggtgtcattctatt | |
| | ctggggggtggggtggggcaggacagcaaggggga | |
| | ggattgggaagagaatagcaggcatgctgggga | |

TABLE 6
Exemplary Guide RNA Sequences

| | | SEQ |
|---|---|---|
| Name | Nucleotide Sequence | ID NO |
| gCCR5_232 | CAATGTGTCAACTCTTGACA | 25 |
| gJCHAIN_2 | GTTAAGGTGTGTGATCTATA | 26 |
| gJCHAIN_31 | AAGAACCATTTGCTTTTCTG | 27 |
| gJCHAIN_54 | TCTCACCACCATATACGAGT | 28 |
| gJtolGHM_23 | GTCTCAGGAGCGGTGTCTGT | 29 |

### REFERENCES

Cheng et al. 2022, “Ex Vivo Engineered Human Plasma Cells Exhibit Robust Protein Secretion and Long-Term Engraftment In Vivo,” Nature Communications.
Hammerland et al. 2017.
Hung et. al., Molecular Therapy, 2018,: https://doi.org/10.1016/j.ymthe.2017.11.012.
Jeske et al. 2021, “Vector Strategies to Actualize B Cell-Based Gene Therapies,” Journal of Immunology.
Jourdan et al. 2009, “An In Vitro Model of Differentiation of Memory B Cells into Plasmablasts and Plasma Cells Including Detailed Phenotypic and Molecular Characterization,” Immunobiology.
Khodadadi et al. 2019, “The Maintenance of Memory Plasma Cells,” Frontiers in Immunology.
Maruo et. al., Trends in Molecular Medicine, 2014, 10.1016/j.molmed.2014.09.003.
Shen et al., 2022, “The Molecular Basis of FIX Deficiency in Hemophilia B,” International Journal of Molecular Sciences.
Sidonio et al., 2021, “Hemophilia B (Factor IX Deficiency),” Hematology/Oncology Clinics of North America.
WO2018/170150, “Engraftable Cell-Based Immunotherapy for Long-Term Delivery of Therapeutic Proteins.”

### EQUIVALENTS

It is to be appreciated by those skilled in the art that various alterations, modifications, and improvements to the present disclosure will readily occur to those skilled in the art. Such alterations, modifications, and improvements are intended to be part of the present disclosure, and are intended to be within the spirit and scope of the invention. Accordingly, the foregoing description and drawing are by way of example only and any invention described in the present disclosure if further described in detail by the claims that follow.

Those skilled in the art will appreciate typical standards of deviation or error attributable to values obtained in assays or other processes described herein. The publications, websites and other reference materials referenced herein to describe the background of the invention and to provide additional detail regarding its practice are hereby incorporated by reference in their entireties.

1. A method of preparing an engineered B lineage cell population that express Factor IX (FIX), the method comprising the steps of:
(a) isolating primary B cells so that a primary B cell population is obtained;
(b) activating the primary B cell population; and
(c) during or after the activating step, engineering the primary B cell population by integrating a transgene encoding FIX at a site selected from a CCR5 site, a IgH site, and a JCHAIN site,
thereby generating an engineered B lineage cell population.

2. The method of claim 1, further comprising a step of expanding the engineered B lineage cell population.

3. The method of claim 1 or 2, further comprising a step of differentiating the engineered B lineage cell population into a population of plasmablasts.

4. The method of claim 3, wherein the differentiation step comprises contacting the engineered B lineage cell population with culture media comprising:
(a) IL-2;
(b) IL-6;
(c) IL-10; and/or
(d) IL-15.

5. The method of claim 3 or 4, further comprising a step of differentiating the population of plasmablasts into a population of plasma cells.

6. The method of claim 5, wherein the differentiation step comprises contacting the population of plasmablasts with culture media comprising:
(a) IL-6;
(b) IL-15; and/or
(c) IFN-alpha-2-beta (IFNα-2β).

7. The method of any one of the above claims, further comprising a step of transferring the engineered B lineage cell population to cell culture media without human or bovine serum for about 24 hours.

8. The method of any one of the above claims, wherein the step of engineering further comprises introducing a donor construct comprising the transgene into the primary B cell population.

9. The method of claim 8, wherein the donor construct comprises:
(a) a 5′ homology arm that is at least 95% identical to a sequence 5′ to the double-stranded break site; and
(b) a 3′ homology arm that is at least 95% identical to a sequence 3′ to the double-stranded break site.

10. The method of claim 9, wherein the 5′ homology arm comprises or is:
(i) a length about 450-850 base pairs in size;
(ii) a PAM site or absence of a PAM site; and/or
(iii) symmetrical or asymmetrical in length with the 3′ homology arm.

11. The method of claim 9 or 10, wherein the 3′ homology arm comprises or is:
(i) a length about 450-850 base pairs in size;
(ii) a PAM site or absence of a PAM site; and/or
(iii) symmetrical or asymmetrical in length with the 5′ homology arm.

12. The method of claim 9, wherein the 5′ homology arm has at least 95% identity with one or more of SEQ ID NOs: 10-13.

13. The method of claim 9, wherein the 3′ homology arm has at least 95% identity with one or more of SEQ ID NOs: 14-18.

14. The method of claim 8, wherein the donor construct comprises a sequence with at least 95% identity with one or more of SEQ ID NOs: 19-21.

15. The method of claim 14, wherein the construct comprises one or more of:
(a) a promoter sequence selected from SEQ ID NOs: 22;
(b) a nucleic acid sequence encoding for Factor IX selected from SEQ ID NOs: 23; and
(c) a polyA sequence selected from SEQ ID NOs: 24.

16. The method of any one of claims 8-15, wherein the donor construct is or comprises an adeno-associated viral (AAV) vector.

17. The method of any one of the above claims, wherein the step of engineering comprises contacting the primary B cell population with a targeted nuclease capable of introducing double-stranded breaks.

18. The method of claim 17, wherein the targeted nuclease is or comprises a CRISPR-associated (Cas) protein, zinc finger nuclease (ZFN), transcription activator-like effector-based nuclease (TALEN), or meganuclease.

19. The method of claim 18, wherein the Cas protein is or comprises Cas9, Cas12a, or Cas13a, or a variant thereof.

20. The method of claim 18, wherein the Cas protein is complexed with a guide RNA (gRNA).

21. The method of claim 20, wherein the gRNA is a single guide RNA (sgRNA).

22. The method of claim 21, wherein the gRNA comprises at least 95% identity with any one of SEQ ID NOs: 25-29.

23. The method of any one of the above claims, wherein the step of engineering comprises:
(a) electroporation of the primary B cell population, and/or
(b) transduction of the primary B cell population with a donor construct.

24. The method of claim 23, wherein the electroporation is performed on a composition comprising:
(a) the Cas protein complexed with the gRNA; and
(b) the primary B cell population.

25. The method of any one of claims 1-24, wherein the CCR5 site has at least 95% identity with SEQ ID NO: 25.

26. The method of any one of claims 1-24, wherein the JCHAIN site has at least 95% identity with one or more SEQ ID NOs: 26-28.

27. The method of any one of claims 1-24, wherein the IgH site has at least 95% identity with SEQ ID NO: 29.

28. The method of any one of claims 1-27, wherein the transgene has at least 95% identity with SEQ ID NO: 23.

29. A population of genetically modified B lineage cells comprising a transgene sequence encoding a FIX protein, wherein the transgene is expressed from an endogenous CCR5 locus.

30. The population of genetically modified B lineage cells of claim 29, wherein the cells express FIX protein and at least partially disrupt expression of endogenous CCR5.

31. A population of genetically modified B lineage cells comprising a transgene sequence encoding a FIX protein, wherein the transgene is expressed from an endogenous JCHAIN locus.

32. The population of genetically modified B lineage cells of claim 31, wherein the cells express FIX protein without disrupting expression of endogenous JCHAIN.

33. A population of genetically modified B lineage cells comprising a transgene sequence encoding a FIX protein, wherein the transgene is expressed from an endogenous IgH locus.

34. The population of genetically modified B lineage cells of claim 33, wherein the cells express FIX protein without disrupting expression of endogenous IgH.

35. The population of any one of claims 29-34, wherein the population of genetically modified B lineage cells is or comprises a population of genetically modified plasma cells.

36. The population of any one of claims 29-34, wherein the population of genetically modified B lineage cells is or comprises a population of plasmablasts.

37. The population of claim 36, wherein the population of genetically modified B lineage cells is or comprises a population of plasma cell precursors.

38. A pharmaceutical composition comprising one or more B lineage cells selected from the population of genetically modified B lineage cells of any of the above claims, and one or more pharmaceutically acceptable excipients.

39. A method of administering a pharmaceutical composition, wherein the pharmaceutical composition comprises:
(a) one or more B lineage cells selected from the population of genetically modified B lineage cells of any one of the above claims; and
(b) one or more pharmaceutically acceptable excipients;
wherein the pharmaceutical composition is administered to a subject.

40. A method of treating a disease, disorder, or condition in a subject, the method comprising administering to the subject a therapeutically effective amount of a pharmaceutical composition of claim 38, thereby treating the disease, disorder, or condition in the subject.

41. The method of claim 39 or 40, wherein the pharmaceutical composition is administered intravenously.

42. The method of any one of claims 39-41, wherein the pharmaceutical composition is administered to an adult subject.

43. The method of any one of claims 39-41, wherein the pharmaceutical composition is administered to a pediatric subject.

44. The method of any one of claims 39-43, wherein the subject has hemophilia B.

45. A method of characterizing a population of genetically modified B lineage cells of any one of claims 29-37, comprising assessing one or more of (a)-(g) using an assay:
(a) presence of a CD38 marker;
(b) presence of a CD138 marker;
(c) presence of at least 50% of a CD27 marker;
(d) secretion of at least 0.5 μg/cell/day of IgG;
(e) secretion of at least 2.5 μg/cell/day of IgM;
(f) secretion of at least 0.5 μg/cell/day of IgD; and
(g) secretion of at least 0.1 μg/cell/day of a FIX protein.

46. The method of claim 45, wherein the assay comprises or is one or more of: fluorescence-activated cell sorting (FAC-sort), Western Blot, flow cytometry, enzyme-linked immunosorbent spot assay (ELISpot), and enzyme-linked immunosorbent assay (ELISA).

47. The method of claim 45 or 46, wherein one or more B lineage cells selected from the population of genetically modified B lineage cells engraft within the bone marrow of the subject.

48. A method of monitoring engraftment of genetically modified B lincage cells within a subject, comprising one or more of: bioluminescence, ELISpot, flow cytometry, and enzyme-linked immunosorbent assay.


SEQUENCE LISTING (29 sequences)

SEQ ID NO: 1 - DNA, 23 bp, synthetic construct
  source 1..23: mol_type = other DNA
gcagcaaaccttcccttcactac

SEQ ID NO: 2 - DNA, 23 bp, synthetic construct
  source 1..23: mol_type = other DNA
aggattcccgagtagcagatgac

SEQ ID NO: 3 - DNA, 22 bp, synthetic construct
  source 1..22: mol_type = other DNA
gggtggaacaagatggattatc

SEQ ID NO: 4 - DNA, 20 bp, synthetic construct
  source 1..20: mol_type = other DNA
catcgcattgtctgagtagg

SEQ ID NO: 5 - DNA, 19 bp, synthetic construct
  source 1..19: mol_type = other DNA
cagtggatcgggtgtaaac

SEQ ID NO: 6 - DNA, 27 bp, synthetic construct
  source 1..27: mol_type = other DNA
tcgggagcctcttgctggaaaatagaa

SEQ ID NO: 7 - DNA, 21 bp, synthetic construct
  source 1..21: mol_type = other DNA
ccacatcagaaggaagactac

SEQ ID NO: 8 - DNA, 19 bp, synthetic construct
  source 1..19: mol_type = other DNA
gctgtatgaatccaggtcc

SEQ ID NO: 9 - DNA, 26 bp, synthetic construct
  source 1..26: mol_type = other DNA
tgtttcctccaggataaggcagctgt

SEQ ID NO: 10 - DNA, 600 bp, synthetic construct
  source 1..600: mol_type = other DNA
ggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgaca

SEQ ID NO: 11 - DNA, 600 bp, synthetic construct
  source 1..600: mol_type = other DNA
tacaaaatacagattttgtgagatatatacaaaatacagattttgtgagatatatacaaaatacagattttgtgagatatatacaaaatacatattttgtgagatatatacaaaatatatatttaaatctggtagcttggttaattaaggactagtataaaaaattaacataagatacacttgggaccttaaaggactttcatttctctccccttccttcatctctcctccttcacttaccttcatttattattctaggcatgacaagtgtcagatgtctgatttgaagactaatatgaatatatctaggtaataaaatggaaaatccctatttcaacattcttaatggaatccttccttgaaattattattagtcttctggtttaaacatttatatagtgtaaatgcaaaataaaaatgattgtatcaaattatggtttttatttggatgctttgcaagaaaatacttaaatgtgagtcatacatttttccagttcagcatttaaacttcagacttcaaccacagttgtgattgtttttagtttgttagctgcctggagtgttattttaagaaagcagaagcaccatcatttgcacact

SEQ ID NO: 12 - DNA, 600 bp, synthetic construct
  source 1..600: mol_type = other DNA
atacatattttgtgagatatatacaaaatatatatttaaatctggtagcttggttaattaaggactagtataaaaaattaacataagatacacttgggaccttaaaggactttcatttctctccccttccttcatctctcctccttcacttaccttcatttattattctaggcatgacaagtgtcagatgtctgatttgaagactaatatgaatatatctaggtaataaaatggaaaatccctatttcaacattcttaatggaatccttccttgaaattattattagtcttctggtttaaacatttatatagtgtaaatgcaaaataaaaatgattgtatcaaattatggtttttatttggatgctttgcaagaaaatacttaaatgtgagtcatacatttttccagttcagcatttaaacttcagacttcaaccacagttgtgattgtttttagtttgttagctgcctggagtgttattttaagaaagcagaagcaccatcatttgcacactccttatagatcacacaccttaaccctgactttttttgctccagtttttcagaagaagtgaagtcaagatgaagaaccatttgctttt

SEQ ID NO: 13 - DNA, 600 bp, synthetic construct
  source 1..600: mol_type = other DNA
ttttctaaagaaaaaatcattggcattcatagaaaggccaatttctcttaatagttcaataagtggatttgatcttataaaaaggcaggtgtttctttggaaatgacagactccaacatcaatttttttaaaaattctccctttcttgtcactataaataacttgtttagacagatatacagttgggaataagcctaacacagtagaaattgctgtatggtgtagataaaacaatcatattatcatatcattaattatattgcttactttcaactaatatatattaaagattggaaaatcccataagctattctgtattgtagagctgcttatgtctgaaaggagtcatcccttgctgtcatgtcagagctgcaagaactaattgattttggattgaaatgtgtagtcacattttgagacagcatttgaggggattgtctaatacatatatttgcttttcagctgtaaaaaatgtgatcctacagaagtggagctggataatcagatagttactgctacccagagcaatatctgtgatgaagacagtgctacagagacctgctacacttatgacagaaacaagtgctacacagctgtggt

SEQ ID NO: 14 - DNA, 450 bp, synthetic construct
  source 1..450: mol_type = other DNA
atgtgacgcccggagacagaaggtctctgggtggctgggtttttgtggggtgaggatggacattctgccattgtgattactactactactactacatggacgtctggggcaaagggaccacggtcaccgtctcctcaggtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgtggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtc

SEQ ID NO: 15 - DNA, 600 bp, synthetic construct
  source 1..600: mol_type = other DNA
ctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaac

SEQ ID NO: 16 - DNA, 600 bp, synthetic construct
  source 1..600: mol_type = other DNA
agatcacacaccttaaccctgactttttttgctccagtttttcagaagaagtgaagtcaagatgaagaaccatttgcttttctggggagtcctggcggtttttattaaggctgttcatgtgaaaggtatgtgatatttagaaaatgatcccagatcaaaggaaaaatataggacagatctgtttttcagttttaaacatttatgcctatgctttggttggccagttagccagctactttttcctaacatgctttcatctttctatggcagttgtctttacatagataagactctttttattcttttttctcttatcttacttacttgtttgcctactacaaactagagaataagaaattagctaactacagatagtcctgcttatatgaatgtccatattgaatcttaaaaatctataatccttcccttgagtctcttaaattaatattctggagaaatgaaataataattactgtaagtacaacatggaatgcattgggattttatttccccaaagaggctggtattggttttaacagctttcctggatctgatcccacagatttattatgaccctttaacaatgtttaatgttataca

SEQ ID NO: 17 - DNA, 600 bp, synthetic construct
  source 1..600: mol_type = other DNA
cgtatatggtggtgagaccaaaatggtggaaacagccttaaccccagatgcctgctatcctgactaatttaagtcattgctgactgcatagctctttttcttgagaggctctccattttgattcagaaagttagcatatttattaccaatgaatttgaaaccagggcttttttttttttttgggtgatgtaaaaccaactccctgccaccaaaataattaaaatagtcacattgttatctttattaggtaatcacttcttaattatatgttcatactctaagtatcaaaatcttccaattatcatgctcacctgaaagaggtatgctctcttaggaatacagtttctagcattaaacaaataaacaaggggagaaaataaaactcaaggactgaaaatcaggaggtgtaataaaatgttcctcgcattcccccccgcttttttttttttttttgactttgccttggagagccagagcttccgcattttctttactattctttttaaaaaaagtttcactgtgtagagaacatatatgcataaacataggtcaattatatgtctccattagaaaaataataattggaaaacatgttctaga

SEQ ID NO: 18 - DNA, 450 bp, synthetic construct
  source 1..450: mol_type = other DNA
tgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctgagaaaaatggttaagaaaattatgacttaaaaatgtgagagattttcaagtatattaatttttttaactgtccaagtatttgaaattcttatcatttgattaacacccat

SEQ ID NO: 19 - DNA, 1392 bp, synthetic construct
  source 1..1392: mol_type = other DNA
gccaccatgcagagggtgaacatgatcatggctgagagccctggcctgatcaccatctgcctgctgggctacctgctgtctgctgagtgcactgtgttcctggaccatgagaatgccaacaagatcctgaacaggcccaagagatacaactctggcaagctggaggagtttgtgcagggcaacctggagagggagtgcatggaggagaagtgcagctttgaggaggccagggaggtgtttgagaacactgagaggaccactgagttctggaagcagtatgtggatggggaccagtgtgagagcaacccctgcctgaatgggggcagctgcaaggatgacatcaacagctatgagtgctggtgcccctttggctttgagggcaagaactgtgagctggatgtgacctgcaacatcaagaatggcagatgtgagcagttctgcaagaactctgctgacaacaaggtggtgtgcagctgcactgagggctacaggctggctgagaaccagaagagctgtgagcctgctgtgccattcccatgtggcagagtgtctgtgagccagaccagcaagctgaccagggctgaggctgtgttccctgatgtggactatgtgaacagcactgaggctgaaaccatcctggacaacatcacccagagcacccagagcttcaatgacttcaccagggtggtggggggggaggatgccaagcctggccagttcccctggcaagtggtgctgaatggcaaggtggatgccttctgtgggggcagcattgtgaatgagaagtggattgtgactgctgcccactgtgtggagactggggtgaagatcactgtggtggctggggagcacaacattgaggagactgagcacactgagcagaagaggaatgtgatcaggatcatcccccaccacaactacaatgctgccatcaacaagtacaaccatgacattgccctgctggagctggatgagcccctggtgctgaacagctatgtgacccccatctgcattgctgacaaggagtacaccaacatcttcctgaagtttggctctggctatgtgtctggctggggcagggtgttccacaagggcaggtctgccctggtgctgcagtacctgagggtgcccctggtggacagggccacctgcctgctgagcaccaagttcaccatctacaacaacatgttctgtgctggcttccatgaggggggcagggacagctgccagggggactctgggggcccccatgtgactgaggtggagggcaccagcttcctgactggcatcatcagctggggggaggagtgtgccatgaagggcaagtatggcatctacaccaaagtctccagatatgtgaactggatcaaggagaagaccaagctgacctga

SEQ ID NO: 20 - DNA, 1392 bp, synthetic construct
  source 1..1392: mol_type = other DNA
gccaccatgcagagggtgaacatgatcatggctgagagccctggcctgatcaccatctgcctgctgggctacctgctgagtgctgagtgcacagtgttcctggaccatgagaatgccaacaagatcctgaacagacccaagagatacaacagtggcaagctggaggagtttgtgcagggcaacctggagagggagtgcatggaggagaagtgcagctttgaggaggccagggaagtgtttgagaacacagagagaaccacagagttctggaagcagtatgtggatggagaccagtgtgagagcaacccttgcctgaatggaggcagctgcaaggatgacatcaacagctatgagtgctggtgcccttttggctttgagggcaagaactgtgagctggatgtgacctgcaacatcaagaatggaaggtgtgagcagttctgcaagaactctgctgacaacaaagtggtgtgtagctgcacagagggctacagactggctgagaaccagaagtcttgtgagccagctgtgccttttccctgtggcagagtcagtgtgtcccagaccagcaagctgaccagagctgagacagtgttccctgatgtggactatgtgaatagcacagaggctgagaccatcctggacaacatcacccagagcacccagtccttcaatgacttcaccagagttgtgggaggggaggatgccaagcctggccagttcccctggcaggtggtgctgaatggcaaagtggatgccttctgtggaggcagcattgtgaatgagaagtggattgtgacagctgcccactgtgtggagacaggagtgaagatcacagtggtggctggagaacacaatattgaggagacagagcacactgagcagaagaggaatgtcatcagaattatcccccaccacaactacaatgctgccatcaacaagtacaaccatgacattgccctgctggagctggatgagcctctggtgctgaatagctatgtgacccccatctgcattgctgacaaggagtacaccaacatcttcctgaagtttggcagtggctatgtcagtggctggggcagagtgttccacaagggcagatctgccctggtgctgcagtacctgagagtgcccctggtggacagagccacctgcctgttgagcaccaagttcaccatctacaacaacatgttctgtgctggcttccatgagggaggcagagacagctgccagggagattctggagggccccatgtgacagaagtggagggcaccagcttcctgacaggcatcatcagctggggggaggagtgtgccatgaagggcaagtatggcatctacaccaaagtcagcagatatgtgaactggatcaaggagaaaaccaagctgacctga

SEQ ID NO: 21 - DNA, 1392 bp, synthetic construct
  source 1..1392: mol_type = other DNA
gccaccatgcagagggtgaacatgatcatggcagagagccctggcctgatcaccatctgcctgctgggctacctgctgtctgcagaatgtacagtgtttctggaccatgagaatgccaacaagatcctgaacaggcccaagagatacaactctggcaagctggaggagtttgtgcagggcaacctggagagggagtgcatggaggagaagtgcagctttgaggaggccagggaagtgtttgagaacactgagaggaccactgagttctggaagcagtatgtggatggagaccagtgtgagagcaacccttgcctgaatggaggcagctgcaaggatgacatcaacagctatgagtgctggtgcccatttggctttgagggcaagaactgtgagctggatgtgacctgcaacatcaagaatggcagatgtgagcagttctgcaagaactctgctgacaacaaagtggtgtgtagctgcactgagggctacagactggcagagaatcagaagtcctgtgaacctgctgtgccttttccctgtggcagagtctctgtgtcccagaccagcaagctgaccagagcagagactgtgttcccagatgtggactatgtgaatagcactgaggcagagaccatcctggacaacatcacccagagcacccagtccttcaatgacttcaccagagttgtgggaggagaggatgccaagccaggccagttcccctggcaggtggtgctgaatggcaaagtggatgccttctgtggaggcagcattgtgaatgagaagtggattgtgacagcagcccactgtgtggagactggagtgaagatcactgtggtggcaggagaacacaatattgaggagactgagcacacagagcagaagagaaatgtcatcaggattatcccccaccacaactacaatgcagccatcaacaagtacaaccatgacattgccctgctggagctggatgagcctctggtgctgaatagctatgtgacccccatctgcattgcagacaaggagtacaccaacatcttcctgaagtttggctctggctatgtcagtggctggggcagagtgttccacaagggcagatctgccctggtgctgcagtacctgagagtgcccctggtggacagagccacctgcctgttgagcaccaagttcaccatctacaacaacatgttctgtgcaggcttccatgagggaggcagagacagctgccagggagactctggaggaccccatgtgactgaagtggagggcaccagcttcctgactggcatcatcagctggggagaggagtgtgccatgaagggcaagtatggcatctacaccaaagtcagcagatatgtgaactggatcaaggagaaaaccaagctgacctga

SEQ ID NO: 22 - DNA, 348 bp, synthetic construct
  source 1..348: mol_type = other DNA
gaacagttggaacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 23 - DNA, 1272 bp, synthetic construct
  source 1..1272: mol_type = other DNA
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 24 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 25 - DNA, 20 bp, synthetic construct
  source 1..20: mol_type = other DNA
caatgtgtcaactcttgaca

SEQ ID NO: 26 - DNA, 20 bp, synthetic construct
  source 1..20: mol_type = other DNA
gttaaggtgtgtgatctata

SEQ ID NO: 27 - DNA, 20 bp, synthetic construct
  source 1..20: mol_type = other DNA
aagaaccatttgcttttctg

SEQ ID NO: 28 - DNA, 20 bp, synthetic construct
  source 1..20: mol_type = other DNA
tctcaccaccatatacgagt

SEQ ID NO: 29 - DNA, 20 bp, synthetic construct
  source 1..20: mol_type = other DNA
gtctcaggagcggtgtctgt